(19) United States
(12) Patent Application Publication
(10) Pub. No.: US 2008/0241240 A1
(43) Pub. Date: Oct. 2, 2008

(54) COMBINED PHARMACEUTICAL FORMULATION WITH CONTROLLED-RELEASE COMPRISING DIHYDROPYRIDINE CALCIUM CHANNEL BLOCKERS AND HMG-COA REDUCTASE INHIBITORS

(75) Inventors: Sung Wuk Kim, Gyeonggi-do (KR); Sung Soo Jun, Gyeonggi-do (KR); Young Gwan Jo, Daejeon (KR); Ja-Seong Koo, Daejeon (KR); Jin Wook Kim, Daejeon (KR); Ju Bin Yim, Chungcheongnam-do (KR); Jun Young Lee, Daejeon (KR)

Correspondence Address: GREENBLUM & BERNSTEIN, P.L.C., 1950 ROLAND CLARKE PLACE, RESTON, VA, 20191, US

(73) Assignee: HANALL Pharmaceutical Co., Ltd., Daejeon (KR)

(21) Appl. No.: 12/038,663

(22) Filed: Feb. 27, 2008

Related U.S. Application Data
(63) Continuation-in-part of application No. PCT/KR2007/004079, filed on Aug. 24, 2007.
Application No. PCT/KR2007/004079 is a continuation-in-part of application No. 11/696,942, filed on Apr. 5, 2007.

(30) Foreign Application Priority Data
Aug. 24, 2006 (KR) 10-2006-0080694
Aug. 24, 2007 (KR) 10-2007-0085480

(51) Int. Cl. A61K 31/44; A61K 31/454; A61K 9/14; A61K 9/22; A61P 9/00

(52) U.S. Cl. 424/468; 514/356; 514/338; 424/489

(57) ABSTRACT

The present invention relates to a chronotherapeutic combination pharmaceutical formulation, which is designed to control the release of each ingredient of the combined drug in a predetermined rate based on the principle of the so-called chronotherapy and xenobiotics, where drugs are administered to exhibit pharmacological activities at predetermined time intervals. The formulations of the present invention comprise a dihydropyridine, and a statin, as active ingredients. The formulations are structured and arranged such that the respective release rates of the above ingredients can be controlled, thereby reducing or preventing antagonistic effects and side effects resulting from the interaction of the above ingredients, while maintaining the synergistic effect, and providing easy medication.

### CROSS-REFERENCE TO RELATED APPLICATIONS

This application is a continuation-in-part of International Application No. PCT/KR2007/004079, filed Aug. 24, 2007, which claims priority from Korean Patent Application No. 10-2006-0080694 filed Aug. 24, 2006, and 10-2007-0085480 filed Aug. 24, 2007; and a continuation-in-part of U.S. Ser. No. 11/696,942, filed Apr. 5, 2007, which claims priority to Korean Patent Application No. 10-2006-0080694. Each of the above U.S., international, and Korean patent applications is incorporated herein by reference in its entirety. If any of the above disclosures conflicts in any way with the present disclosure, the present disclosure shall be deemed to control.

### FIELD OF THE INVENTION

The present invention relates to a chronotherapeutical combination pharmaceutical formulation with a programmed predetermined delayed release (or controlled-release) component comprising a dihydropyridine, calcium channel blocker as an active ingredient, and a rapid release (or instant release) component comprising a statin, a lipid-lowering agent, as an active ingredient. The formulation of the present invention is designed in such a manner that the release of each ingredient can be controlled at a predetermined rate by using the so-called chronotherapy and xenobiotics, where drugs are administered to exhibit the pharmacological activities at certain time intervals thereby providing improved therapeutic effects while minimizing side effects.

### BACKGROUND

A person afflicted with both atherosclerosis and hypertension generally deteriorates more quickly, and has worse symptoms, than a person with only one of these conditions. These can be prevented only by treatment of both atherosclerosis and hypertension at the same time in patients suffering from hyperlipidemia and hypertension. Hypertens. Res. 2001; 24: 3-11, Hypertens. Res. 2003; 26: 1-36, Hypertens. Res. 2003; 26: 979-990 (incorporated by reference herein in their entireties).

Thus, there have been clinical results reported about the synergistic effect of the co-administration of a statin as a lipid-lowering agent and a calcium channel blocker as the anti-hypertensive According to Kramsch et al., a combination of amlodipine and a lipid-lowering agent shows a better therapeutic effect for atherosclerosis. J. of Human Hypertension (1995) (Suppl. 1), 53-59 (incorporated by reference herein in its entirety). Jukema et al. has proved the synergistic effect when a calcium channel blocker and a lipid-lowering agent are administered in a combined dosage form. Circulation, 1995 (Suppl. 1), 1-197 (incorporated by reference herein in its entirety).

A combination of amlodipine as a representative calcium channel blocker, particularly a dihydropyridine, a calcium channel blocker, along with simvastatin as a statin, a lipid-lowering agent, is most widely prescribed.

It is well known that amlodipine serves as a medication for treatment of angina as well as hypertension. Simvastatin is also well known to have a lipid-lowering activity and an anti-inflammatory activity on the wall of blood vessels. The safety of these drugs, particularly of simvastatin, are also well known and are available in a drug store in Great Britain as over-the-counter (OTC) drugs not requiring prescription. Cardiology 1992; 80 (Suppl. 1): S31-S36, J. Cardiovasc. Pharmacol. 1988; 12 (Suppl. 7): S110-S113, Lancet 2000; 356: 359-365, Hypertens. Res. 2002; 25: 717-725, Hypertens. Res. 2002; 25: 329-333; Am J Manag Care. 2004 Oct. 10 (Suppl. 11):S332-8; Curr Control Trials Cardiovasc Med 2000, 1:161-165 (incorporated by reference herein in their entireties).

Meanwhile, amlodipine serves as an anti-hypertensive medicine and also increases the lipid-lowering activity of simvastatin through synergistic activity with the lipid-lowering agent. Simvastatin serves as a lipid-lowering agent and also has an activity of decreasing blood pressure through a synergistic effect with amlodipine. The aforementioned two drugs are both administered once daily, and the medication for both drugs is preferred to be administered with dinner.

As a representative statin, a lipid-lowering agent, simvastatin has the following characteristics. That is, it is well known that a statin HMG-CoA reductase inhibitor as a lipid-lowering agent is the first option for prevention and treatment of heart diseases due to coronary atherosclerosis, angina or myocardial infarction. Lancet 1995; 346: 750-753, Am J Cardiol 1998; 82: 57T-59T, Am J Cardiol 1995; 76: 107C-112C, Hypertens Res 2003; 26: 699-704, Hypertens Res 2003; 26: 273-280; Br Med Bull 2001; 59: 3-16, Am J Med 1998; 104 (Suppl 1): 6S-8S, Clin Pharmacokinet 2002; 41: 343-370 (incorporated by reference herein in their entireties).

Moreover, simvastatin is most frequently prescribed among statins, lipid-lowering agents, and has been well known to decrease the prevalence of coronary atherosclerosis and the death rate through a large-scale clinical test. Lancet 1994; 344: 1383-1389 (incorporated by reference herein in its entirety).

The aforementioned activities are due to the fact that simvastatin strongly inhibits HMG-CoA reductase, which plays a key role in the synthesis of cholesterol in the liver and also inhibits an inflammation-inducing factor. “Scandinavian Simvastatin Survival Study” published in the Lancet, 1994, 344, 1383-89 (incorporated by reference herein in its entirety).

Patients suffering from atherosclerosis or diabetes show abnormal NO synthase (eNOs) in blood vessel wall, and the blood pressure increases due to the decrease in NO generation. Statin, a lipid-lowering agent including simvastatin, increases the e-NOS to a normal level, which is also an effect of a combined prescription where a lipid-lowering activity helps an anti-hypertensive activity. Am. J. Physiol. Renal. Physiol. Vol. 281 Issue 5: F802-F809, 2001 (incorporated by reference herein in its entirety).

Among the more important hepatic enzymes, there are the xenobiotic metabolizing enzymes. Upon absorption from the gastrointestinal tract, drug molecules are first sent via the portal vein to the liver. In the liver, xenobiotic metabolizing enzymes, such as the cytochrome P450 oxidases, have an opportunity to act on newly-absorbed drug molecules, as well as metabolites thereof. In some cases, xenobiotic metabolizing enzymes deactivate the drug. In some other cases, however, xenobiotic metabolizing enzymes convert the absorbed drug to an active form from an inactive (or less active) form. Without being bound by any particular theory, it has surprisingly been found that xenobiotic metabolizing enzymes, cytochrome P450A oxidases (e.g., cytochrome P450A 3A4), interact with statins and dihydropyridines in very different ways, such that administering a statin and a dihydropyridine to a person in need thereof at arbitrary times relative to each other can result harmful side effects (e.g. rhabdomyolysis), or in ineffectiveness of one or both components.

Statins comprising lactone rings are generally metabolized by enzymes, e.g., hepatic enzymes, e.g., cytochrome P450 3A4, which open the lactone ring and form an active metabolite, a β-hydroxy acid. For example, simvastatin is an inactive lactone, which, after entering liver, is transformed into the β-hydroxy acid activated form, also known as simvastatin acid, with lipid-lowering activity. The remaining simvastatin is also metabolized through Various steps by cytochrome P450 3A4 in the liver, and some of metabolites show a strong lipid-lowering activity.

Statins and their metabolites, e.g., simvastatin and simvastatin β-hydroxy acid, are metabolized by cytochrome P450 3A4, functions in liver and excreted from liver. Drug Metab Dispos 1990; 18: 138-145, Drug Metab Dispos 1990; 18: 476-483, Drug Metab Dispos 1997; 25: 1191-1199; Drugs 1997; 53:828-847; Clin Pharmacokinet 1996; 5:348-371; Arch Biochem Biophys 1991; 290:355-361; Drug Metab Dispos 1997, 25:321-331; Drug Metab Dispos. March 1999, 27(3):410-6. (incorporated by reference herein in their entireties).

Thus, when used in combination with a drug inhibiting cytochrome P-450 enzymes, statins are subject to less metabolism in liver and the plasma concentration of the statin HMG-CoA reductase inhibitor is increased, which may lead to serious side effects such as rhabdomyolysis. Clin Pharmacol Ther 1998; 63: 332-341, Clin Pharmacol Ther 1998; 64:177-182, Physicians Desk Reference 2006 (Zocor), J Pharmacol Exp Ther 1997; 282: 294-300, Pharmacol Exp Ther 1999; 290: 1116-1125, Life Sci. 2004, 76: 281-292; Drug Metab Dispos 1991; 19:740-748; Eur J Clin Pharmacol 1996, 50:209-215 (incorporated by reference herein in their entireties).

Therefore, a very specially designed administration should be employed in the co-administration of such drugs like amlodipine (or other dihydropyridine) that inhibits the induction of the cytochrome P450 enzymes, together with such drugs like simvastatin (or other statins) that should be metabolized to the active form by the same cytochrome P450 enzymes. Besides, statin-based drugs have been recommended to be administered early in the evening because lipid synthesis in the liver becomes very active from the evening. Arterioscler. Thromb. 11: 816-826, Clinic. Pharmacol. Ther. 40: 338-343; Am J. Cardiol. 2006 Jan. 1, 97(1):44-7, Epub 2005 Nov. 8 (incorporated by reference herein in their entireties).

Dihydropyridine calcium channel blockers are an anti-hypertensive agent, which prevents calcium from flowing into vascular smooth muscle and induces the relaxation of peripheral arteries, thereby lowering the blood pressure. Also, dihydropyridines are effective drugs for treating the ischemic diseases owing to spasmodic contraction of coronary arterial walls.

A representative calcium channel blocker, amlodipine, is known as follows.

A calcium channel blocker is the anti-hypertensive medicine that is most frequently prescribed in combination with simvastatin. Particularly, amlodipine is the most widely prescribed in the world as an anti-hypertensive medicine and a medicine for coronary arterial ischemic diseases like angina. Cardiology 1992; 80 (Suppl 1): S31-S36, J. Cardiovasc. Pharmacol. 1988; 12 (Suppl 7): S110-S113, Lancet 2000; 356: 359-365, Hypertens. Res. 2002; 25: 717-725, Hypertens. Res. 2002; 25: 329-333 (incorporated by reference herein in their entireties).

Amlodipine, which is used in the present invention in combination with statin, a lipid-lowering agent represented by simvastatin, is a long-acting drug belonging to dihydropyridine, calcium channel blocker. Clin. Pharmacokinet. 1992; 22: 22-31, Am. Heart J 1989; 118: 1100-1103, Hypertens. Res. 2003; 26: 201-208 (incorporated by reference herein in their entireties).

Amlodipine, which has a chemical name of 3-ethyl-5-methyl-2-(2-amino ethoxymethyl)-4-(2-chlorophenyl)-1,4-dihydro-6-methyl-3,5-pyridinedicarboxylate, is a very useful calcium channel blocker that has a half-life of 30-50 hours and shows an activity for a relatively long period of time. European patent publication No. 89,167 and U.S. Pat. No. 4,572,909 (incorporated by reference herein in their entireties). Amlodipine is also a medicine for treating hypertension, which prevents calcium from flowing into vascular smooth muscle and induces the relaxation of peripheral arteries, thereby lowering blood pressure. Also, amlodipine is a useful drug for treating ischemic diseases caused by spasmodic contraction of coronary arterial walls.

When orally administered in the form of a single pill, amlodipine is absorbed in small intestine. Then, more than 40% is metabolized in the liver and only the remaining 60% is present in blood, thus sufficiently exerting an activity of lowering blood pressure.

Amlodipine continues the activity for 24 hours, and shows strongest activity of lowering blood pressure during the time from morning to noon when administered in the evening of the previous day.

From a pathophysiological point of view, the high pressure increase in the daytime, e.g., in the morning is caused by the spasm of vascular wall due to stress stimulus. Amlodipine functions by causing the relaxing of the spasmodic contraction of vascular wall, and shows a strong activity of lowering blood pressure in the day time, e.g., morning. Thus, amlodipine administered during the evening of the previous day reaches the maximum plasma concentration in the early morning and shows strong activity during the time of morning stress. Clin. Invest. 72: 864-869 (incorporated by reference herein in its entirety).

In the presence of cytochrome P450 3A4 enzyme, some amlodipine is inactivated. However, amlodipine also rapidly acts to inhibit generation of cytochrome P450 enzymes. Dihydropyridines are mainly metabolized by cytochrome P450 3A4 in addition metabolized by other cytochrome P450 enzymes (e.g. cytochrome P450 2C9).

Due to the aforementioned nature, dihydropyridine calcium channel blockers should be administered at certain time intervals when co-administered with a statin, a lipid-lowering agent such as simvastatin, because amlodipine inhibits the cytochrome P450 3A4 enzyme needed by simvastatin. Med. Chem. 1991; 34: 1838-1844, Eur J. Clin. Pharmacol. 2000; 55: 843-852 (incorporated by reference herein in their entireties). That is, when a patient is to take both a statin and dihydropyridine, they should be administered in such a way as to allow for a time gap separating when the statin and dihydropyridine are absorbed from the gastrointestinal tract.

The co-administration of amlodipine and simvastatin has had such delicate problems as mentioned above, but so far it has been very difficult to formulate such combination products free of such problems. It is today's tendency that the prescription of the co-administration of amlodipine and Simvastatin has been continuously practiced by physicians but rarely being found that appropriate instructions for medication would usually be given to patients. Therefore, the majority of patients do not know that simvastatin at least should be taken in the evening and amlodipine should be taken after an interval of a certain number of hours.

However, the co-administration of the aforementioned drugs may increase the plasma concentration of simvastatin acids by about 30% thus generating side effects. It is also difficult to expect the synergistic effects of two drugs in lowering blood pressure and lipid.

Shinichiro Nishio et al. (Hypertens. Res., 2005, Vol. 28, No. 3, pp. 223-227) (incorporated by reference herein in its entirety) reported the result of experiments comparing two groups of patients suffering from hypertension with hyperlipidemia. One group was administered with amlodipine single pill and simvastatin single pill at the same time, and the other group was administered with simvastatin single pill only.

According to the experiments, the simultaneous co-administration of simvastatin and amlodipine inhibits cytochrome P450 3A4 enzyme due to amlodipine and increases the plasma concentration of simvastatin by 30% leading to the possibility of side effects.

TABLE 1

| Daily dosage | Cₘₐₓ (ng/mL) | AUC (ng · h/mL) |
|---|---|---|
| Simvastatin 5 mg | 9.6 ± 3.7 | 34.3 ± 16.5 |
| Simvastatin 5 mg + | 13.7 ± 4.7 | 43.9 ± 16.6 |
| Amlodipine 5 mg | | |

Hypertension Research Vol. 25 (2005). No. 3 March 223-227

As shown in Table 1, as compared to the administration of simvastatin only, the combined prescription was higher by 30% in the plasma concentration of the lipid-lowering metabolites. Nevertheless, the lipid-lowering activity was not increased. At a higher plasma concentration than a certain level, simvastatin decreases in activity of inhibiting biosynthesis of cholesterol, and is likely to incur serious side effects such as rhabdomyolysis.

Korean patent No. 582347 (incorporated by reference herein in its entirety) discloses a combination formulation where the amlodipine is dissolved and absorbed at the same time that the statin-based component is simultaneously released for 24 hours. The concept of this patent is completely in the opposite way against the concept of the present invention. However this formulation (Korean patent No. 582347) definitely shows such defect that the two drug components are simultaneously mingled in liver from the first time of the dissolution and an antagonistic drug interaction occurs; that is, amlodipine inhibits the induction of cytochrome P450 3A4 enzyme needed by simvastatin. As a result, some simvastatin β-hydroxy acids exit the liver and enter the bloodstream before being completely metabolized by cytochrome P450 3A4. Increasing the plasma concentration of simvastatin can unnecessarily bring about the side effects such as rhabdomyolysis.

Korean patent No. 742432 (incorporated by reference herein in its entirety) discloses a combination formulation comprising amlodipine camsylate and simvastatin, and a manufacturing method thereof. In this patent, the two ingredients are released and metabolized in a liver simultaneously. Such formulation is a simple combination where two drug components are simultaneously dissolved, absorbed and entering into the liver. So amlodipine inhibits the induction of cytochrome P450 3A4 to be needed by simvastatin for its activation and complete metabolism in the liver. As a result about 30% of simvastatin and its metabolites are released into blood without acting completely and being metabolized in liver. Such unnecessary higher concentration of simvastatin and its metabolites does not contribute to lowering lipid but increasing side effects. Such defect of simple combination has been ascertained by preliminary clinical test disclosed in the present invention.

Further, Korean patent publication No. 2000-7002144 by Pfizer (incorporated by reference herein in its entirety) was also rejected by Korean Intellectual Property Office as being a simple formulation containing amlodipine and atorvastatin.

Thus, there are needs for developing a novel pharmaceutical formulation method or a novel pharmaceutical formulation that may prevent shortcomings, e.g., undesirable drug interactions and/or side effects, of the conventional techniques, i.e., such conventional combination products comprising a dihydropyridine (e.g., amlodipine) and a statin-based drug in that these two components are either simultaneously absorbed, or the dihydropyridine (e.g., amlodipine) is absorbed before absorbed before the statin.

### SUMMARY OF THE INVENTION

Therefore, there has been a long felt need for the development of a novel medication method or a novel pharmaceutical formulation that may prevent the drawback of the combination product of two drug components, i.e., the antagonistic effects between the drugs.

As a result, a specially combination pharmaceutical formulation that may overcome the antagonistic effects between drugs was developed in the present invention by taking advantage of the pharmaceutical concept that the drugs may be dissolved at prescheduled intervals.

The present inventors have exerted extensive researches to develop a way to solve the aforementioned problems and increase the therapeutic effect of the combined prescription, which is clinically inevitable, while reducing the side effects.

A clinical test herein proves that the chronotherapeutic administration of a statin, a lipid-lowering agent represented by simvastatin, and a dihydropyridine, a calcium channel blocker represented by amlodipine, has remarkably improved therapeutic effect and safety as compared to that of the simultaneous co-administration of the drugs.

The present invention has found that a time interval between the absorption of a statin, a lipid-lowering agent, and a dihydropyridine, a calcium channel blocker, in the gastrointestinal tract may inhibit any higher plasma concentration of the statin, a lipid-lowering agent than are necessary and prevent various relevant side effects thereof, and developed a combined formulation herein, thereby finally completing the present invention.

In the case of a combination system of the present invention, statin, lipid-lowering agent is absorbed first rapidly after the administration of the formulation and transformed into an activated form, by cytochrome P450 enzymes, and acts in the liver and then further metabolized by cytochrome P450 enzymes to be eliminated through bile duct. After enough time from when simvastatin is affected by cytochrome P450 enzymes in the liver, amlodipine is absorbed in the gastrointestinal tract. Therefore simvastatin may not be affected by the inhibitory action of amlodipine on the induction of cytochrome P450 enzymes.

Considering that simvastatin enters the liver as a first-pass, and is metabolized into active form to inhibit the cholesterol synthesis in the liver, the present invention is intended to formulate the combination products in such a way that simvastatin may sufficiently stay in the liver for its full lipid lowering activity but not being released into the blood with a higher plasma concentration than a certain level. For this purpose the present invention is characterized in its specially designed controlled releasing formulation.

That is, the present invention is related to control of the controlled-release ingredients by constituting the formulation comprising a delayed-release portion containing a dihydropyridine, a calcium channel blocker, and an immediate-release part containing a statin, a lipid-lowering agent, as active ingredients, thus enabling the dihydropyridine, a calcium channel blocker, to be dissolved or absorbed in the small intestine 3-4 hours later than that of the statin.

As compared with the simply formulated combination product of two drug components (calcium channel blocker/statin tablet), the chronotherapeutically combination pharmaceutical formulation of the present invention comprising a dihydropyridine, a calcium channel blocker, and a statin, a lipid-lowering agent, is expected to show far superior clinical effects and safety.

The combination products of the present invention when orally administered, provide a synergistic effect of the two drug components (calcium channel blocker/statin) in the clinical effect and the safety by maximizing the pharmaceutical activity of each active ingredient, by minimizing the antagonistic effects of two drugs in the liver and through the control of release of each drug components to maintain its absorption with the lapse of time. For such purpose, the combination product of the present invention is to be administered one tablet daily in the evening preferably between 5 pm and 10 pm.

In principle, a drug should not be administered in combination with another drug if the co-administration results in more harm than benefit.

Considering the great synergistic clinical effects of a combined administration of a dihydropyridine calcium channel blocker and a statin lipid-lowering agent, such as amlodipine and simvastatin, the present invention maintains the synergistic effects and also eliminates the side effects by resolving the side effect of simvastatin, e.g., myopathy, which may appear when the two drugs simply being co-administered. Such antagonistic relationship between amlodipine and simvastatin is based on the simple fact that each drug is related to the same cytochrome P 450 3A4 in such antagonistic way that unnecessary simvastatin acids are increased in the blood.

Therefore, the present invention aims to provide a functional chronotherapeutic combination pharmaceutical formulation comprising a controlled-release dihydropyridine, calcium channel blocker and a statin, lipid-lowering agent.

The present invention provides a combined pharmaceutical formulation comprising a dihydropyridine, a calcium channel blocker, and a statin, a lipid-lowering agent, as active ingredients and a pharmaceutically acceptable carrier, the combined pharmaceutical formulation including a delayed part comprising the dihydropyridine, a calcium channel blocker, and a rapid-release part comprising the statin, a lipid-lowering agent, as active ingredients.

As mentioned herein, the present invention applies the chronotherapeutic theory and xenobiotic theory in the controlled release formulation to maximize the pharmacokinetic and pharmacodynamic effects and to minimize the side effects, which may occur when two drugs are simply co-administered.

The formulation of the present invention comprises as active ingredients, statin, a lipid-lowering agent, and dihydropyridine, a calcium channel blocker, and both of ingredients are related to the same cytochrome P450 enzyme, in such a manner that one is affecting on while the other is affected by that enzyme, and is characterized in that the release rates of the aforementioned ingredients are different and the dissolution and absorption of each drug are initiated at certain intervals of time in a controlled manner.

A pharmaceutical composition according to the present invention may comprise a delayed release component comprising at least one dihydropyridine and a rapid release component comprising at least one statin.

As a result, the formulation of the present invention is more useful pharmacologically, clinically, scientifically and economically in the treatment of a chronic circulatory disorder than when the two drugs are simply co-administered

Moreover, the combined pharmaceutical formulation of the present invention causes the drugs to be released at different rates, and prevents the antagonistic effects and side effects, while maintaining the synergistic effect of the drugs.

Further, the combined pharmaceutical formulation of the present invention is administered with a single dose once daily in the evening, and has an advantage of convenience in medication and medication instruction.

The present invention includes a chronotherapeutic combination pharmaceutical formulation with controlled-release comprising a controlled-release part containing a dihydropyridine, a calcium channel blocker, and an immediate-release part containing statin, a lipid-lowering agent, as active ingredients. The present invention includes methods of using and administering the formulations.

The present invention also includes a method of administering to a patient in need thereof at least one statin and at least one dihydropyridine, the method comprising administering to the patient a formulation comprising a therapeutically effective amount of at least one statin and a therapeutically effective amount of at least one dihydropyridine, wherein the statin is absorbed, and after a time interval, the dihydropyridine is absorbed.

The present invention also includes a method of preventing or treating hypertension, angina pectoris, atherosclerosis, arteriosclerosis, complex hypertension, hyperlipidemia, hypercholesterolemia, myocardial infarction, cardioplegia, heart failure or ischemic heart disease, comprising administering to a patient in need thereof a pharmaceutical composition comprising a delayed release component comprising at a therapeutically effective amount of least one dihydropyridine and a rapid release component comprising a therapeutically effective amount of at least one statin.

The present invention also includes a method of administering a drug combination comprising administering to a subject in need thereof a therapeutically effective amount of at least one statin and a therapeutically effective amount of at least one dihydropyridine wherein the statin is in a rapid-release form and the dihydropyridine is in a delayed-release form.

The present invention also includes a method of delivering a statin and a dihydropyridine with a time differential to the liver of a subject, the method comprising administering to a subject in need thereof a therapeutically effective amount of at least one statin and a therapeutically effective amount of at least one dihydropyridine wherein the statin is in a rapid-release form and the dihydropyridine is in a delayed-release form.

The present invention also includes a method of reducing interference between a statin and a dihydropyridine in a subject in need thereof comprising administering to the subject a therapeutically effective amount of at least one statin and a therapeutically effective amount of at least one dihydropyridine wherein the at least one statin is in a rapid-release form and the at least one dihydropyridine is in a delayed-release form.

Preferably, the release property of the dihydropyridine, calcium channel blocker and the statin, lipid-lowering agent are controlled so that the dihydropyridine, calcium channel blocker is dissolved to be absorbed in the liver 3-4 hours later than the statin, lipid-lowering agent.

Preferably, the release property of the dihydropyridine, calcium channel blocker and the statin, lipid-lowering agent are controlled to decrease the interaction between the two drug ingredients metabolized at the same time in the liver via cytochrome P450 that the dihydropyridine, calcium channel blocker is released 2 hours after the onset of the release of the statin, lipid-lowering agent.

Preferably, the dihydropyridine calcium channel blocker comprises at least one of amlodipine, lercanidipine, lacidipine, felodipine, barnidipine, benidipine, cilnidipine, isradipine, manidipine, nicardipine, nifedipine, nimodipine, nilvadipine, nisoldipine, nitrendipine, or a pharmaceutically acceptable salt or ester thereof, preferably at least one of amlodipine, lercanidipine, lacidipine, or a pharmaceutically acceptable salt, ester, or isomer thereof, preferably amlodipine besylate or amlodipine mesylate or amlodipine maleate.

Preferably, the statin lipid-lowering agent comprises at least one of simvastatin, lovastatin, atorvastatin, pitavastatin, rosuvastatin, fluvastatin, pravastatin, or a pharmaceutically acceptable salt or ester thereof, preferably at least one of simvastatin, lovastatin, atorvastatin, or a pharmaceutically acceptable salt, ester, or isomer thereof.

Preferably, the formulation comprises 1-400 mg, preferably 1-240 mg of the dihydropyridine. Preferably, the formulation comprises 1-500 mg, preferably 1-160 mg of the statin. Unit dosage forms (e.g., tablets or capsules) may comprise this amount of dihydropyridine and/or statin or, e.g., about one-half or about one-third of these amounts.

Preferably, the timed-release part comprises a release controlling material comprising at least one of an enteric polymer, a water-insoluble polymer, a hydrophobic compound, a hydrophilic non-polymeric compound, a hydrophilic polymer, an osmotic semi-permeable membrane coating base, or an osmogant.

Preferably, the release controlling material in the controlled-release part is contained in an amount of 10-3,000 weight parts relative to 100 weight parts of the dihydropyridine calcium channel blocker.

When used, the enteric polymer preferably comprises at least one of polyvinylacetate phthalate, methacrylic acid copolymer, hydroxypropylmethyl cellulose phthalate, shellac, cellulose acetate phthalate, cellulose propionate phthalate, poly(methacrylate-methyl methacrylate) copolymer (e.g., Eudragit L), or poly(methacrylate-ethyl acrylate) copolymer (e.g., Eudragit S).

When used, the water-insoluble polymer preferably comprises a polyvinylacetate, methacrylic acid copolymer comprising at least one of poly(ethylacrylate-co-methylmethacrylate) copolymer or poly(ethylacrylate-methyl methacrylate-trimethyl aminoethyl methacrylate) copolymer, ethyl cellulose, or cellulose acetate.

When used, the hydrophobic compound preferably comprises at least one of a fatty acid, a fatty acid ester, a fatty acid alcohol, a wax, or an inorganic material.

When used, the fatty acid and fatty acid esters preferably comprise at least one of glyceryl palmitostearate, glyceryl stearate, glyceryl behenate, cetyl palmitate, glyceryl mono oleate, stearic acid and a mixture thereof; the fatty acid alcohol is selected from the group consisting of cetostearyl alcohol, cetyl alcohol, stearyl alcohol and a mixture thereof; the wax is selected from the group consisting of carnauba wax, beeswax, microcrystalline wax and a mixture thereof, the inorganic material is selected from the group consisting of talc, precipitated calcium carbonate, dibasic calcium phosphate, zinc oxide, titanium oxide, kaolin, bentonite, montmorillonite, veegum and a mixture thereof.

When used, each of the hydrophilic non-polymeric compound and the hydrophilic polymer preferably comprises at least one of a saccharide, a cellulose derivative, a gum, a protein, a polyvinyl derivative, a polymethacrylate copolymer, a polyethylene derivative, or a carboxyvinyl polymer.

When used, the saccharide preferably comprises at least one of dextrin, polydextrin, dextran, pectin and pectin derivative, alginate, poly(galacturonic acid), xylan, arabinoxylan, arabinogalactan, starch, hydroxypropyl starch, amylose, amylopectin and a mixture thereof; the cellulose derivative preferably comprises at least one of hydroxypropylmethyl cellulose, hydroxypropyl cellulose, hydroxymethyl cellulose, hydroxyethyl cellulose, methyl cellulose, carboxymethyl cellulose sodium, hydroxypropyl methyl cellulose acetate succinate, or hydroxyethylmethyl cellulose; the gum preferably comprises at least one of guar gum, locust bean gum, tragacanth, carrageenan, gum acacia, gum arabic, gellan gum, or xanthan gum; the protein preferably comprises at least one of gelatin, casein, or zein; the polyvinyl derivative preferably comprises at least one of polyvinyl alcohol, polyvinyl pyrrolidone, or poly(vinylacetal diethylaminoacetate); the polymethacrylate copolymer preferably comprises at least one of a poly(butyl methacrylate-(2-dimethylaminoethyl)methacrylate-methyl methacrylate) copolymer, a poly(methacrylic acid-methyl methacrylate) copolymer, or a poly(methacrylic acid-ethyl acrylate) copolymer; the polyethylene derivative preferably comprises at least one of polyethylene glycol, or polyethylene oxide; and the carboxyvinyl polymer preferably comprises carbomer.

When used, the osmotic semi-permeable membrane coating base preferably comprises at least one of a polyvinylacetate, poly(ethylacrylate-co-methylmethacrylate) copolymer, poly(ethylacrylate-methylmethacrylate-trimethylaminoethylmethacrylate) copolymer, ethyl cellulose cellulose acetate, cellulose ester, cellulose ether, cellulose acylate, cellulose diacylate, cellulose triacylate, cellulose acetate, cellulose diacelate, or cellulose triacetate.

When used, the osmogant preferably comprises at least one of a magnesium sulfate, magnesium chloride, sodium chloride, lithium chloride, potassium sulfate, sodium sulfate or lithium sulfate.

The pharmaceutical formulation can comprise a pill or tablet with a structure of two-phased matrix, wherein the controlled-release part is placed discontinuously, thereby causing the controlled-release of the dihydropyridine, a calcium channel blocker, and the immediate-release part is placed continuously, thereby causing the immediate-release of the statin, a lipid-lowering agent. The formulation according to the present invention may be prepared into biphasic matrix partitioned in a single pill by granule phase, multi-layered tablet, press-coated tablet or a capsule filled with granules of a delayed part and an immediate-release part.

The controlled-release part and the immediate-release part can form a multi-layered structure. Preferably, the pharmaceutical formulation comprises a single pill or tablet with a double-layered structure comprising an inner core of the controlled-release part and an outer layer of the immediate-release part, which encompasses, encloses, or contacts the inner core.

The pharmaceutical formulation can comprise a capsule. A capsule can comprise the at least one dihydropyridine, and the at least one statin. A capsule can comprise granules of the controlled-release component and/or granules of the rapid release component. A capsule can comprise pellets of the delayed release component and/or pellets of the rapid release component. A capsule can comprise one or more tablets of the delayed release component, and/or one or more tablets of the rapid release component. A tablet can comprise a pressed tablet and/or an osmotic pump. A capsules can comprise combinations of one or more of granules, pellets or tablet. The delayed release component can comprise an enteric polymer.

The formulation can comprise an uncoated tablet or a coated tablet. A coated tablet preferably comprises at least one of a coating layer of a film former, or a film-forming adjuvant. The coating layer preferably comprises at least one of a cellulose derivative, a saccharide derivative, a polyvinyl derivative, a wax, fat, gelatin, polyethylene glycol, ethyl cellulose, titanium oxide, or diethyl phthalate. The coating layer is preferably contained in an amount of 0.5-15 wt % of the total weight of the coated tablet.

Preferably, the pharmaceutical formulation of the present invention is administered in the evening or night, preferably at about 5-10 p.m., preferably at bedtime.

Preferably, the statin is released first and the dihydropyridine is gradually released 2 hours after administration, thereby decreasing interaction between the statin, a lipid-lowering agent, and the dihydropyridine, a calcium channel blocker.

### BRIEF DESCRIPTION OF THE DRAWINGS

FIG. 1 shows a graph comparing dissolution rates between the amlodipine/simvastatin press coated matrix tablets prepared in Example 1 and the control drugs (Zocor®: simvastatin single pill, Norvasc®: amlodipine single pill).

FIG. 2 shows a graph comparing dissolution rates between the amlodipine/simvastatin combination pharmaceutical formulation prepared in Examples 4 and 10 and the control drugs (Zocor®: simvastatin single pill, Norvasc®: amlodipine single pill).

FIG. 3 shows a graph comparing dissolution rates between the amlodipine/lovastatin combination pharmaceutical formulation prepared in Example 11 and the control drugs (Mevacor®: lovastatin single pill, Norvasc®: amlodipine single pill).

FIG. 4 shows a graph comparing dissolution rates between the amlodipine/atorvastatin combination pharmaceutical formulation prepared in Example 13 and the control drugs (Lipitor®: atorvastatin single pill, Norvasc®: amlodipine single pill).

FIG. 5 shows a graph comparing dissolution rates between the lercanidipine/simvastatin combination pharmaceutical formulation prepared in Example 16 and the control drugs (Zocor®: simvastatin single pill, Zanidip®: lercanidipine single pill).

FIG. 6 shows a graph comparing dissolution rates between the lacidipine/simvastatin combination pharmaceutical formulation prepared in Example 18 and the control drugs (Zocor®: simvastatin single pill, Vaxaar®: lacidipine single pill).

FIG. 7 is a graph comparing dissolution rates between the amlodipine besylate/simvastatin combination pharmaceutical formulation prepared in Example 20 and the control drugs (Zocor®: simvastatin single pill, Norvasc®: amlodipine single pill).

FIG. 8 is a graph comparing dissolution rates between the capsules containing amlodipine besylate/simvastatin combination tablets prepared in Example 22 and the control drugs (Zocor®: simvastatin single pill, Norvasc®: amlodipine single pill).

FIG. 9 is a graph comparing dissolution rates between the amlodipine besylate/atorvastatin combination pharmaceutical formulation prepared in Example 30 and the control drugs (Lipitor®: atorvastatin single pill, Norvas®: amlodipine single pill).

FIG. 10 is a graph comparing dissolution rates between biphasic capsules containing the felodipine/atorvastatin combination pharmaceutical formulation prepared in Example 36 and the control drugs (Lipitor®: atorvastatin single pill, Munubal®: felodipine single pill).

FIG. 11 is a graph comparing dissolution rates between biphasic capsules containing the isladipine/fluvastatin combination pharmaceutical formulation prepared in Example 40 and the control drugs (Lescol®: fluvastatin single pill, Dynacirc®: isladipine single pill).

FIG. 12 is a graph comparing dissolution rates of (S)-amlodipine/simvastatin combination pharmaceutical formulation prepared in Example 51 and 55.

FIG. 13 is a graph comparing dissolution rates of (S)-amlodipine/simvastatin combination pharmaceutical formulation prepared in Example 52, 53 and 54.

FIG. 14 shows results of clinical test in Experimental Example 14, which compares the plasma concentrations of simvastatin β-hydroxy acid between experimental groups.

FIG. 15 shows results of clinical test in Experimental Example 14, which compares the plasma concentrations of simvastatin and simvastatin β-hydroxy acid between experimental groups.

FIG. 16 shows results of clinical test in Experimental Example 14, which compares the plasma concentrations of amlodipine between experimental groups.

### DETAILED DESCRIPTION OF THE INVENTION

Hereunder is provided a detailed description of the present invention.

The present invention relates to a chronotherapeutic combined pharmaceutical formulation, which is designed such that the release of each ingredient may be controlled to a predetermined rate by applying the principle of the so-called chronotherapy and the xenobiotics, where drugs are administered so that the activities of the drugs are effective chronotherapeutically without any antagonistic interaction of drugs. The formulation of the present invention comprises as active ingredients, a statin, a lipid-lowering agent, and a dihydropyridine, a calcium channel blocker, both of which are related to the same cytochrome P450 enzyme, in such a manner that one is affecting on while the other is affected by that enzyme, and is characterized in that the release rates of the aforementioned ingredients are different and the dissolution and absorption of each drug are initiated at certain intervals of time in a controlled manner, thereby preventing antagonistic effects and side effects, while maintaining the synergistic effect and providing convenience in medication.

Hereunder is provided a detailed description of the combined pharmaceutical formulation according to the present invention, which comprises a dihydropyridine, calcium channel blocker and a statin, lipid-lowering agent.

The combined pharmaceutical formulation of the present invention comprises a dihydropyridine, a calcium channel blocker, and a statin, a lipid-lowering agent as active ingredients. Such a dihydropyridine, a calcium channel blocker, is known as a substance inhibiting the induction of cytochrome P450 3A4.

Any dihydropyridine effective at lowering blood pressure may be used in the compositions and methods of the present invention. Without limitation, some examples of dihydropyridine calcium channel blockers include amlodipine, lercanidipine, lacidipine, felodipine, barnidipine, benidipine, cilnidipine, isradipine, manidipine, nicardipine, nifedipine, nimodipine, nivadipine, nisoldipine, and nitrendipine. The dihydropyridine may be in the form of the free base, or a pharmaceutically acceptable salt thereof (e.g., besylate, mesylate, or hydrochloride salt), and combinations of two or more thereof are also included. In a combination of two or more dihydropyridines, it is the amount of the combination (not necessarily the individual components) that should effective. A preferred dihydropyridine is amlodipine or a pharmaceutically acceptable salt thereof or an isomer or ester thereof, more preferably amlodipine maleate and/or amlodipine besylate.

Any dosage level of the dihydropyridine that is effective may be used, and those of ordinary skill in the art understand that different dihydropyridines may have different optimal dosage levels, which can depend on the patient and the indication for which the drug is taken. Generally, therapeutically effective dosage levels can be expected to be between about 1-400 mg. Preferable daily dosage of the dihydropyridine, calcium channel blocker of the present invention, e.g., of amlodipine besylate, is in the range of about 1-20 mg, preferably about 5-10 mg, for, e.g., a male adult human of weight 65-75 kg. All amounts are based on weight of free base.

As the dihydropyridine, calcium channel blocker having an efficacy of lowering blood pressure, the present application specifically describes amlodipine. However, the present invention shall not be limited to amlodipine.

Any lipid lowering drug, preferably a statin, may be used in the compositions and methods of the present invention. Without limitation, some examples of statins that can be used include simvastatin, lovastatin, atorvastatin, pitavastatin, rosuvastatin, fluvastatin, pravastatin, cilastatin, nystatin, pentostatin and isomers thereof. The statin may be in the form of the free base, or a pharmaceutically acceptable salt thereof (e.g., sodium or calcium salt), and combinations of two or more thereof are also included. In a combination of two or more statins, it is the total amount of the combination (not necessarily the individual components) that should be therapeutically effective. Preferred statins include simvastatin, lovastatin, atorvastatin and isomers thereof.

Any dosage level of the statin that is effective may be used, and those of ordinary skill in the art understand that different statins may have different optimal dosage levels, which can depend on the patient and the indication for which the drug is taken. A therapeutically effective daily dosage of the statin, lipid-lowering agent in the present invention will generally be in the range of about 1-60 mg. A preferred daily dosage of the statin is about 2-80 mg for an adult human.

In a preferred embodiment, the dosage form is provided as a tablet, preferably having total weight in the range of about 200-500 mg.

Representative example of the statin, a lipid-lowering agent, is simvastatin, and the present invention describes simvastatin as a specific example. However, the present invention is in no way limited to simvastatin. Although simvastatin is inactive material, it may be changed into an active simvastatin acid by esterase, and further changed into an activated form by cytochrome P450 3A4 in the liver, thereby exerting a lipid-inhibiting activity.

Meanwhile, amlodipine inhibits the induction of enzyme cytochrome P450 3A4. Therefore, when amlodipine and simvastatin are administered at the same time, amlodipine that is rapidly absorbed into the small intestine reaches liver earlier than simvastatin and thereby inhibits the induction of cytochrome P450 3A4. Hence, a considerable portion of the simvastatin that reaches the liver later or at the same time is not subject to the metabolic activity of cytochrome P450 3A4 and about 30% of the simvastatins (simvastatin, simvastatin acids, etc.) may enter the systemic bloodstream before they are fully metabolized by cytochrome P450 3A4 to be active in the liver or excreted through the bile duct. As a result, the necessary high concentration of simvastatin or simvastatin acid in the blood may cause muscular disorder like rhabdomyolysis.

As a way to solve the aforementioned problem and prevent amlodipine from inhibiting the full enzymatic metabolic pathway of simvastatin in the liver, the present invention constitutes an immediate-release part that releases simvastatin first and causes simvastatin to be absorbed by the small intestine earlier, while constituting a delayed release or controlled-release part amlodipine to be absorbed into liver 34 hours later than that of simvastatin. As used herein, the terms delayed release and controlled release are interchangeable.

Formulations and methods of the present invention reduce or eliminate the simultaneous presence in the liver and/or blood stream of statin (or metabolite thereof) and dihydropyridine. Any time interval between release and/or absorption of the components sufficient to accomplish this is suitable. It should be understood that a certain amount of overlap between presence of statin (or metabolite, e.g., β-hydroxy acid metabolite, thereof) and dihydropyridine is acceptable, but the overlap should not be so great as to cause interference as discussed herein.

Accordingly, dosage forms of the present invention provide for a difference in absorption times of a statin and a dihydropyridine. Preferably, dosage forms of the present invention include a rapid release component containing a drug to be released first, and a delayed release component, containing a drug to be released second. The first release drug preferably comprises at least one statin. The delayed release component preferably comprises at least one dihydropyridine.

The rapid release component may begin release of the first drug, e.g., a statin, at any time after ingestion. Release of the first drug (and absorption thereof) may begin immediately after ingestion, or after a brief delay. In this regard, it is to be understood that the term “rapid-release” as used herein refers to rapidity of onset of release of the drug from the dosage form, and not necessarily to the speed with which the drug is released. The first drug will preferably be at least about 80% released (and absorbed), more preferably at least about 90% released, more preferably, about 100% released after about 0.5 or 1 hour. Testing for drug release is preferably done according to the protocol described below.

The delayed release component may begin release of the second drug, e.g., a dihydropyridine, with a sufficient delay after ingestion to avoid interference, e.g., in the liver, with absorption or metabolism of the first drug. The second drug (and absorption thereof) will preferably be less than about 40% released, more preferably less than about 30%, 20% or 10% released, more preferably about 0% released about 2 or 4 hours after ingestion. The amount of dihydropyridine is preferably less than about 40% or 30% of total dihydropyridine after about 3 hours. Testing for drug release is preferably done according to the protocol described below.

Viewed in another way, there is preferably a time interval between substantial completion of release (substantial completion of absorption and first-pass transport to the liver) of the first drug and the beginning of release (beginning of absorption and first-pass transport to the liver) of the second drug. The time interval may be of any length of time sufficient to reduce, minimize, or prevent, competing effects on drug metabolism in the liver, and is preferably at least long enough to prevent simultaneous first-pass delivery of both first and second drugs to the liver. Even a very brief time interval may be sufficient for the present invention. Preferably, the time interval is at least about 2, 3 or 4 hours.

The time interval may be gauged in vitro according to the methods in the Examples, below, or by any equivalent method that yields the same results. In particular, the testing takes place in two stages, intended to simulate gastric conditions followed by intestinal conditions. Thus, the formulation may be tested according to the Examples, with a change in dissolution medium after two hours from artificial gastric juice to artificial intestinal juice. The first stage proceeds according to the general dissolution test method described in Korea Pharmacopoeia (8th revision); test method: paddle method, 75 rpm; dissolution medium: 750 mL of 0.01 M hydrochloric acid for two hours. The second stage begins at two hours, when the dissolution medium is changed to 1000 mL of artificial intestinal fluid (pH=6.8).

Any type of dosage form may that permits the differential release of statin and calcium channel blocker may be used in the present invention. Without limitation, such dosage forms may include osmotic devices; capsules encapsulating tablets, pellets, granules, powders, or a combination of thereof; differential-release tablets; or a plurality of dosage units, such as a kit containing separate dosage forms of the statin and dihydropyridine.

Pellets for use in the present invention may be made in any way by those of ordinary skill in the art. Preferably, pellets comprise a core comprising sugar (e.g., sucrose or lactose), starch, or cellulose. A composition comprising a solution or suspension of drug (e.g., statin or dihydropyridine) is preferably applied in any of several methods, such as with a fluidized bed coater or a pan coater. The composition comprising the drug may contain other excipients, e.g., fillers, film-forming polymers, enteric polymers, plasticizers, etc., as desired.

Granules for use in the present invention may be prepared in any suitable way by those of ordinary skill in the art. In a preferred method, granules may be prepared by wet granulation, a technique well-known to those of ordinary skill in the art. In general, wet granulation includes mixing a drug with one or more excipients, and applying a solution or suspension comprising a polymeric binder and possibly other excipients. The wet composition is then generally dried and sized. Sieving is a referred method of sizing dried granulates.

Without limitation, an osmotic device within the present invention might include a delayed inner core with an active ingredient (e.g., a dihydropyridine) comprising an osmotic device, which is coated with a rapid release coating comprising an active ingredient (e.g., a statin); or might include a two-component pump that releases each active ingredient at different times.

Osmotic devices and methods of making them are well-known. As is known to those of skill in the art, osmotic device dosage forms comprise a semi-permeable membrane enclosing a composition comprising an active ingredient, and may optionally also comprise an osmotic agent. Examples of osmotic semi-permeable membrane coating materials include, but are not limited to, polyvinylacetate, poly(ethylacrylate-co-methylmethacrylate) copolymer, poly(ethylacrylate-methylmethacrylate-trimethylaminoethylmethacrylate) copolymer, ethyl cellulose cellulose acetate, cellulose ester, cellulose ether, cellulose acylate, cellulose diacylate, cellulose triacylate, cellulose acetate, cellulose diacelate, cellulose triacetate and mixture of two or more thereof. Examples of osmotic agents (or osmogants) include, but are not limited to, magnesium sulfate, magnesium chloride, sodium chloride, lithium chloride, potassium sulfate, sodium sulfate lithium sulfate and mixture of two or more thereof.

Without limitation, a capsule (e.g., a hard or soft gelatin capsule) within the present invention might include a combination of rapid release and delayed release pellets; a combination of rapid release and delayed release tablets; a combination of a delayed release tablet and a rapid release powder; or other combinations. Without limitation, a tablet within the present invention might comprise a delayed release core coated with a rapid release coat; or a multilayered (e.g., bilayered or trilayered) tablet comprising a delayed release layer and a rapid release layer. Without limitation, a kit might comprise a blister pack into which a delayed release dosage unit, and a rapid release dosage unit are packed, e.g., with a rapid release dosage unit and a delayed release dosage unit included in each blister, e.g., with instructions to administer each component of the combination at the same time. In each of these dosage forms, the delayed release component preferably comprises a calcium channel blocker as an active ingredient, and the rapid release component preferably comprises a statin as an active ingredient.

Moreover, a tablet for oral administration having a structure of a time release layer as an inner core and an immediate-release layer encompassing the inner core may be obtained by mixing and compressing the granules contained in the time release part with a pharmaceutically acceptable additive to provide an press-coated tablet and by mixing and compressing the granules contained in the immediate-release part with a pharmaceutically acceptable additive.

Thus, formulations of the present invention may be unitary compositions (i.e., contain both statin and dihydropyridine in a single unit dosage form), or may comprise each active ingredient in a separate unit dosage form (e.g., a rapid release statin tablet and a delayed release dihydropyridine capsule). Unit dosage forms (e.g., tablet or capsule) preferably comprise an effective amount of active ingredient, but may comprise sub-therapeutic amounts. In the latter case, multiple dosage units are preferably administered (e.g., two tablets or capsules), such that the patient is administered therapeutically effective amounts of the active ingredients.

The composition of the present invention can comprise a controlled-release composition containing amlodipine, a pharmaceutically acceptable salt thereof and desired additives and an immediate-release composition containing simvastatin and desired additives, which is physically separated or partitioned so that two different drugs show different release rates. Moreover, the immediate-release part and the controlled-release part may be formulated into various forms.

That is, the pharmaceutical composition may be coated according to a conventional method by using a release controlling material selected among the group comprising the controlled-release part, and thus obtained coated particles or granules and multi-component particles or granules of an immediate-release simvastatin composition may be compressed into a tablet or filled in a capsule.

The controlled-release part of the present invention contains a dihydropyridine, a calcium channel blocker such as amlodipine, and an enteric polymer, a water-insoluble polymer, a hydrophobic compound, a hydrophilic nonpolymeric compound and a hydrophilic polymer as a release controlling material thereof. The release controlling material in the controlled-release part may be contained in an amount of 10-3,000 weight parts relative to 100 weight parts of the dihydropyridine, calcium channel blocker. If the amount is below the above range, the release control may not be sufficient. If the amount is above the range, the release of drug is delayed and statistically significant clinical effect may not obtained.

Examples of the enteric polymer include but are not limited to polyvinylacetate phthalate, methacrylic acid copolymer, hydroxypropylmethyl cellulose phthalate, shellac, cellulose acetate phthalate, cellulose propionate phthalate, Eudragit L, Eudragit S and a mixture thereof may be used.

Examples of the water-insoluble polymer include but are not limited to a pharmaceutically acceptable polyvinylacetate, methacrylic acid copolymer such as poly(ethylacrylate-co-methylmethacrylate) copolymer or poly(ethylacrylate-methyl methacrylate-trimethyl aminoethyl methacrylate) copolymer, ethyl cellulose, cellulose acetate and a mixture thereof may be used.

Examples of the hydrophobic organic compound include but are not limited to a fatty acid and a fatty acid ester, a fatty acid alcohol, a wax, an inorganic material and a mixture thereof may be used. Specifically, examples of the fatty acid and fatty acid esters include but are not limited to glyceryl palmitostearate, glyceryl stearate, glyceryl behenate, cetyl palmitate, glyceryl mono oleate, stearic acid and a mixture thereof may be used; examples of the fatty acid alcohol include but are not limited to cetostearyl alcohol, cetyl alcohol, stearyl alcohol and a mixture thereof; examples of the wax include but are not limited to Carnauba wax, beeswax, microcrystalline wax and a mixture thereof; and the examples of the inorganic material include but are not limited to talc, precipitated calcium carbonate, dibasic calcium phosphate, zinc oxide, titanium oxide, kaolin, bentonite, montmorillonite, veegum and a mixture thereof.

Examples of the hydrophilic polymers include but are not limited to saccharides, cellulose derivatives, gums, proteins, polyvinyl derivatives, polymethacrylate copolymers, polyethylene derivatives, carboxyvinyl polymers and mixtures thereof. Specifically, examples of the saccharide include but are not limited to dextrin, polydextrin, dextran, pectin and pectin derivative, alginate, poly(galacturonic acid), xylan, arabinoxylan, arabinogalactan, starch, hydroxypropyl starch, amylase, amylopectin and a mixture thereof; examples of the cellulose derivative include but are not limited to hydroxypropylmethyl cellulose, hydroxypropyl cellulose, hydroxymethyl cellulose, hydroxyethyl cellulose, methyl cellulose, carboxymethyl cellulose sodium, hydroxypropyl methyl cellulose acetate succinate, hydroxyethylmethyl cellulose and a mixture thereof, examples of the gums include but are not limited to guar gum, locust bean gum, tragacantha, carrageenan, gum acasia, gum arabic, gellan gum, xanthan gum and a mixture thereof; examples of the proteins include but are not limited to gelatin, casein, zein and a mixture thereof; examples of the polyvinyl derivative include but are not limited to polyvinyl alcohol, polyvinyl pyrrolidone, polyvinylacetal diethylaminoacetate and a mixture thereof, examples of the polymethacrylate copolymer include but are not limited to poly(butyl methacrylate, (2-dimethylaminoethyl)methacrylate, methylmethacrylate) copolymer, poly(methacrylic acid, methylmethacrylate) copolymer, poly(methacrylic acid, ethylacrylate) copolymer and a mixture thereof; examples of the polyethylene derivative include but are not limited to polyethylene glycol, polyethylene oxide and a mixture thereof; and examples of the carboxyvinylpolymer include but are not limited to carbomer.

The formulation of the present invention may further comprise such amounts of other additives that the effect of the present invention may not be damaged. Examples of a pharmaceutically acceptable diluent as the aforementioned additives include without limitation starch, microcrystalline cellulose, lactose, glucose, mannitol, alginate, salt of alkaline earth metal, clay, polyethylene glycol and dicalcium phosphate. Examples of a lubricant as the aforementioned additives include without limitation talc, magnesium stearate and alkaline earth metal stearate type calcium, zinc, etc., lauryl sulfate, hydrogenated vegetable oil, sodium benzoate, sodium stearyl fumarate, glyceryl monostearate and polyethylene glycol 4000.

The controlled-release part of the present invention consists of discontinuous phases of particles or granules prepared by mixing, granulating or coating a dihydropyridine, as a calcium channel blocker, a release controlling material and commonly used pharmaceutical additives.

The rapid release part of the present invention may be prepared into particles or granules by performing normal processes for manufacture of oral solid forms such as mixing, combining, drying and granulation using statin, lipid-lowering agent such as simvastatin as an active ingredient and a pharmaceutically acceptable additive. If the flowability of simvastatin mixture is good enough for direct compression, the mixture may be mixed to provide composition, while if the fluidity is not good, a composition may be prepared by compaction, granulation and grinding, thus enabling to prepare a continuous phase comprising an immediate-release part.

A formulation for oral administration comprising a controlled-release part and an immediate-release part matrix in two phases by post-mixing a composition contained in the controlled-release part and the immediate-release part with pharmaceutically acceptable additives for compression or by filling the composition into a capsule.

For example, the formulation according to the present invention may be prepared into two-phase matrix partitioned in a single pill by granule phase, multi-layered tablet, inner core tablet or a capsule filled with granules of a controlled-release part and an immediate-release part. Moreover, the formulation may also be prepared into a tablet comprising a controlled-release inner core tablet containing amlodipine and an immediate-release double inner core tablet containing simvastatin.

However, the formulation according to the present invention is not limited to a single two-phase matrix tablet where a discontinuous phase of a controlled-release amlodipine exists in a continuous phase of an immediate-release simvastatin.

That is, a tablet for oral administration having layers for an immediate-release or a controlled-release by mixing granules contained in the controlled-release part and the immediate-release part with pharmaceutically acceptable additives, followed by compression into a double-layered or a triple-layered tablet where layers are parallel to each other using a compressor for the production of a multi-layered tablet.

Moreover, a tablet for oral administration having a structure of a controlled-release layer as an inner core and an immediate-release layer encompassing the inner core by mixing and compressing the granulates contained in the controlled-release part with pharmaceutically acceptable additive to provide an inner core tablet and by mixing and compressing the granulates contained in the immediate-release part with a pharmaceutically acceptable additive.

Further, a capsule formulation for oral administration, where the two-phase release-control is possible, can be obtained by mixing granulates contained in the controlled-release part and the immediate-release part with a pharmaceutically acceptable additive and filling the mixture in a capsule.

Examples of the pharmaceutical additives that constitute the controlled-release part of the formulation herein include a diluent, a binder, a disintegrant, a lubricant, a stabilizer, a colorant and a flavor. Preferable amount of these additives is 100-3,000 weight parts with regard to 100 weight parts of the statin, lipid-lowering agent. Besides the active ingredient and the release controlling material, the formulation of the present invention may further comprise such amounts of other additional ingredients that the effect of the present invention may not be damaged. Examples of a pharmaceutically acceptable diluent as the aforementioned additives include without limitation starch, microcrystalline cellulose, lactose, glucose, mannitol, alginate, a salt of alkaline earth metal, clay, polyethylene glycol and dicalcium phosphate. Examples of binder as the aforementioned additives include without limitation starch, microcrystalline cellulose, highly-dispersed silica, mannitol, lactose, polyethylene glycol, polyvinylpyrrolidone, hydroxypropyl methyl cellulose, hydroxypropyl cellulose, natural gum, synthetic gum, copovidone and gelatin. Examples of a disintegrant as the aforementioned additives include without limitation starch or denatured starch such as sodium starch glycolate, corn starch, potato starch and pre-gelatinated starch; clay such as bentonite, montmorillonite and veegum; celluloses such as microcrystalline cellulose, hydroxypropyl cellulose and carboxymethyl cellulose; aligns such as sodium alginate or alginic acid; crosslinked celluloses such as croscarmellose sodium; gums such as guar gum and xanthan gum; a crosslinked polymer such as crospovidone; and effervescent formulation such as sodium bicarbonate and citric acid. Examples of a lubricant as the aforementioned additives include without limitation talc, magnesium stearate and alkaline earth metal stearate type calcium, zinc, etc., lauryl sulfate, hydrogenated vegetable oil, sodium benzoate, sodium stearyl fumarate, glyceryl monostearate and polyethylene glycol 4000. Other pharmaceutically acceptable additives such as coloring agents or perfumery may be used.

Although microcrystalline cellulose, sodium starch glycolate, colloidal silicon dioxide, magnesium stearate, etc are used in Examples herein as the additives, the present invention is in no way limited to the aforementioned additives, and the usage of the additives may be easily determined by one skilled in the art.

The formulation may optionally comprise a coating layer on the surface of the tablet. That is, the amlodipine/simvastatin combined pharmaceutical formulation of the present invention may be formulated into an uncoated form or a coated tablet for better stability of active ingredients.

The coating layer may be formed on the surface of tablet using the aforementioned ingredients by conventional methods such as a fluidized-bed coating method and, preferably, a pan coating method.

The coating layer may be prepared by using a film former, a film-forming adjuvant or a mixture thereof. In particular, the coating layer may be prepared by using cellulose derivative such as hydroxypropylmethyl cellulose and hydroxypropyl cellulose, saccharide derivative, polyvinyl derivative, waxes, lipids, gelatin and a mixture as a film former; polyethylene glycol, ethyl cellulose, glycerides, titanium oxide, diethyl phthalate and a mixture thereof as a film-forming adjuvant.

The coating layer is preferred to be contained in an amount of 0.5-15 wt % of total weight of the coated tablet.

The combined pharmaceutical formulation of the present invention is prepared into a single combined pharmaceutical formulation containing amlodipine and simvastatin as active ingredients, and may be administered once daily in the evening.

Hence, comparing with the case of the co-administration of two drugs simultaneously or the case of the administration of each drug separately with time intervals, the combined pharmaceutical formulation of the present invention has advantages of easy medication instructions, providing the benefit to minimize the side effects and reduced efficacy to be caused by the antagonistic effects between the drugs.

When administered orally, the combined pharmaceutical formulation of the present invention shows an immediate-release of simvastatin and releases more than 80% of initial amount of simvastatin within one hour. The release of amlodipine is sufficiently controlled in a small intestine, which begins 2 hours after the administration, and the amount of release until 3 hours after the administration does not exceed 40% of initial amount of amlodipine. It is preferable that simvastatin releases more than 90% of initial amount within one hour, and that the released amount of amlodipine does not exceed 30% of initial amount until 3 hours after the administration.

Further, the present invention discloses results of a clinical study, which compares the therapeutic effects of (i) a simultaneous co-administration of commercially available statins, lipid-lowering agents and commercially available dihydropyridine calcium channel blockers (5 mg of amlodipine besylate) as a control group, and (ii) a chronotherapeutic administration of commercially available statins, lipid-lowering agents and dihydropyridine calcium channel blockers as a test group. The chronotherapeutic administration was designed so that the release rates of the drugs are the same as in a combined formulation in the present invention.

As a result, it was ascertained that the chronotherapeutic group shows a remarkable improvement in therapeutic efficacy and safety than the simultaneous co-administration group, and that such therapeutic efficacy and safety seemed to be partially due to the changes in the plasma concentration of drugs and partially due to the full synergistic effects of two drugs.

The present inventors have exerted extensive researches to find effective methods to formulate the combination product of the present invention.

The combined pharmaceutical formulation of the present invention may be used for prevention and treatment of hypertension, atherosclerosis, hyperlipidemia, cerebro-cardiovascular ischemic diseases, stroke, renal ischemic disease, etc.

### EXAMPLES

The present invention is described more specifically by the following Examples. Examples herein are meant only to illustrate the present invention, but they should not be construed as limiting the scope of the present invention.

### Example 1

### Preparation of Amlodipine-Simvastatin Press-Coated Tablets

1) Preparation of Amlodipine Controlled-Release Layer

Predetermined amounts of amlodipine maleate and microcrystalline cellulose as shown in Table 2 were sieved with a No. 35 sieve, and mixed using a double cone mixer. The mixture was placed into a fluidized-bed granulator (GPCG 1: Glatt), and sprayed with a binder solution (an aqueous solution of hydroxypropylmethyl cellulose) to prepare granules, and dried. The granules were combined with carbomer 71G powders, and mixed with magnesium stearate with a double cone mixer. The resulting mixture was compressed using a rotary compressor (MRC-33: Sejong) at a speed of 30 revolutions per minute (rpm) to provide tablets with a hardness of 7-9 kp, a thickness of 3.0 mm and a diameter of 5.5 mm.

2) Preparation of Simvastatin Rapid Release Layer

Predetermined amounts of simvastatin, microcrystalline cellulose and mannitol as shown in Table 2 were sieved with a No. 35 sieve and mixed using a high-speed mixer. A binder solution prepared by dissolving hydroxypropyl cellulose and citric acid in water, and combined with the mixture containing the main ingredients. Thus obtained mixture was granulated using an oscillator with a No. 20 sieve, dried at 60° C. using a steam dryer, ground with a No. 20 sieve. The resulting mixture was mixed with butylatedhydroxyanisole, sodium starch glycolate and colloidal silicon dioxide, and finally mixed with magnesium stearate using a double cone mixer.

3) Compression and Coating

The compositions were compressed using a compressor for the production of press-coated tablet (KUD-1: Kilian) by using the amlodipine core tablet as inner layer and the composition containing simvastatin as outer layer, respectively. The compression was performed under such conditions that the interlayer incorporation may be minimized at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea), thus producing press-coated tablet.

### Example 2

### Preparation of Amlodipine-Simvastatin Biphasic Tablets

1) Preparation of Amlodipine Delayed Release Granules Predetermined amounts of amlodipine and microcrystalline cellulose as shown in Table 2 were sieved with a No. 35 sieve and mixed. The mixture was mixed using Kollicoat SR30D in a high-speed mixer. Thus obtained mixture was granulated using oscillator with a No. 20 sieve, dried at 60° C. using a steam dryer and sized with a No. 20 sieve.

2) Preparation of Simvastatin Rapid Release Granules

Predetermined amounts of simvastatin, microcrystalline cellulose and mannitol as shown in Table 2 were sieved with a No. 35 sieve and mixed using a high-speed mixer. A binder solution was prepared by dissolving hydroxypropyl cellulose and citric acid in water and combining with the mixture of the main ingredients. Thus obtained mixture was combined, granulated using an oscillator with a No. 20 sieve, dried at 60° C. using a steam dryer, ground with a No. 20 sieve, and mixed with butylatedhydroxyanisole.

3) Post-Mixing, Compression and Coating

The obtained composition was mixed using a double cone mixer, added with sodium starch glycolate and colloidal silicon dioxide, and finally mixed with magnesium stearate.

The final composition was compressed using a rotary compressor (MRC-33: Sejong) at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea), thus producing biphasic (two-phase) matrix tablets.

### Example 3

### Preparation of Amlodipine-Simvastatin Biphasic Tablets

1) Preparation of Amlodipine Delayed Release Granules

Predetermined amounts of amlodipine maleate and microcrystalline cellulose as shown in Table 2 were sieved with a No. 35 sieve, and mixed using a double cone mixer. The mixture was placed into a fluidized-bed granulator (GPCG 1: Glatt), and sprayed with a binder solution (an aqueous solution of hydroxypropylmethyl cellulose) to prepare granules. After the granules were dried, they were coated by spraying a 5 wt % solution of ammonio methacrylate copolymer (Eudragit RS PO) in a 1:1 mixture of ethanol and methylene chloride.

2) Preparation of Simvastatin Rapid Release Granules

Predetermined amounts of simvastatin, microcrystalline cellulose and mannitol as shown in Table 2 were sieved with a No. 35 sieve and mixed using a high-speed mixer. A binder solution was prepared by dissolving hydroxypropyl cellulose and citric acid in water and combining with the mixture of the main ingredients. Thus obtained mixture was granulated using an oscillator with a No. 20 sieve, dried at 60° C. using a steam dryer, ground with a No. 20 sieve, and mixed with butylatedhydroxyanisole.

3) Post-Mixing, Compression and Coating

The obtained composition was mixed using a double cone mixer, added with sodium starch glycolate and colloidal silicon dioxide, and mixed with magnesium stearate using a high-speed mixer.

The final composition was compressed using a rotary compressor (MRC-33: Sejong) at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea), thus producing two-phase matrix tablets.

### Example 4

### Preparation of Amlodipine-Simvastatin Multi-Layered Tablets

1) Preparation of Amlodipine Controlled-Release Layer

Predetermined amounts of amlodipine and microcrystalline cellulose as shown in Table 2 were sieved with a No. 35 sieve, and mixed using a double cone mixer. The mixture was sprayed with a binder solution (an aqueous solution of hydroxypropylmethyl cellulose) to prepare granules. After the granules were dried, they were coated by spraying a 5 wt % solution of hydroxypropylmethyl cellulose phthalate in a 1:1 mixture of ethanol and methylene chloride. The coated granules were mixed with magnesium stearate using a double cone mixer.

2) Preparation of Rapid Release Simvastatin Layer

Predetermined amounts of simvastatin, microcrystalline cellulose and mannitol as shown in Table 2 were sieved with a No. 35 sieve and mixed using a high-speed mixer. A binder solution was prepared by dissolving hydroxypropyl cellulose and citric acid in water and combining with the mixture of the main ingredients. Thus obtained mixture was combined, granulated using an oscillator with a No. 20 sieve, dried at 60° C. using a steam dryer and ground with a No. 20 sieve. The granules were mixed with butylatedhydroxyanisole, sodium starch glycolate and colloidal silicon dioxide, and finally mixed with magnesium stearate using a double cone mixer.

3) Compression and Coating

The composition was compressed using a compressor for the production of a multi-layered tablet (MRC-37: Sejong). In detail, the composition comprising simvastatin was input in a first power inlet and the composition comprising amlodipine was input in a second inlet. The compression was performed under such a condition that the interlayer incorporation may be minimized at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea), thus producing multi-layered tablets.

### Example 5

Preparation of Amlodipine-Simvastatin Two-Phase Matrix Tablets

1) Preparation of Amlodipine Controlled-Release Granules

Predetermined amounts of amlodipine and microcrystalline cellulose as shown in Table 2 were sieved with a No. 35 sieve, and mixed using a double cone mixer. The mixture was placed into a fluidized-bed granulator (GPCG 1: Glatt), and sprayed with a binder solution: (an aqueous solution of hydroxypropylmethyl cellulose) to prepare granules. After drying, the granules were coated by spraying a 5 wt % solution of hydroxypropylmethyl cellulose phthalate in a 1:1 mixture of ethanol and methylene chloride.

2) Preparation of Simvastatin Granules for Rapid Release

Predetermined amounts of simvastatin, microcrystalline cellulose and mannitol as shown in Table 2 were sieved with a No. 35 sieve and mixed using a high-speed mixer. A binder solution was prepared by dissolving hydroxypropyl cellulose and citric acid in water, and combined with the mixture of the main ingredients. The combined mixture was granulated using an oscillator with a No. 20 sieve, dried at 60° C. using a steam dryer, ground with a No. 20 sieve, and mixed with butylatedhydroxyanisole.

3) Post-Mixing, Compression and Coating

The obtained composition was mixed using a double cone mixer, added with sodium starch glycolate and colloidal silicon dioxide, and mixed with magnesium stearate using a high-speed mixer.

The final composition was compressed using a rotary compressor (MRC-33: Sejong) at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea), thus producing two-phase matrix tablets.

### Example 6

### Preparation of Amlodipine-Simvastatin Two-Phase Matrix Tablets

1) Preparation of Amlodipine Controlled-Release Layer

Predetermined amounts of amlodipine and microcrystalline cellulose as shown in Table 2 were sieved with a No. 35 sieve, and mixed using a double cone mixer. The mixture was sprayed with a binder solution (an aqueous solution of hydroxypropylmethyl cellulose) to prepare granules. After the granules were dried, they were coated by spraying a 5 wt % solution of ammonio methacrylate copolymer (Eudragit RS PO) in a 1:1 mixture of ethanol and methylene chloride. The coated granules were mixed with magnesium stearate using a double cone mixer.

2) Preparation of Simvastatin Layer for Rapid Release

Predetermined amounts of simvastatin, microcrystalline cellulose and mannitol as shown in Table 2 were sieved with a No. 35 sieve and mixed using a high-speed mixer. A binder solution was prepared by dissolving hydroxypropyl cellulose and citric acid in water, and combining with the mixture of the main ingredients in a high-speed mixer. Thus obtained mixture was granulated using an oscillator with a No. 20 sieve, dried at 60° C. using a steam dryer and ground with a No. 20 sieve. The granules were mixed with butylatedhydroxyanisole, sodium starch glycolate and colloidal silicon dioxide, and finally mixed with magnesium stearate using a double cone mixer.

3) Compression and Coating

The composition was compressed using a compressor for the production of a multi-layered tablet (MRC-37: Sejong). In detail, the composition comprising simvastatin was input in a first power inlet and the composition comprising amlodipine was input in a second inlet. The compression was performed under such a condition that the interlayer incorporation may be minimized at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea), thus producing multi-layered tablets.

### Example 7

### Preparation of Amlodipine-Simvastatin Press-Coated Tablets

1) Preparation of Amlodipine Core Tablet

Predetermined amounts of amlodipine and microcrystalline cellulose as shown in Table 0.2 were sieved with a No. 35 sieve, and mixed using a double cone mixer. The mixture was introduced into a fluidized-bed granulator (GPCG 1: Glatt) and sprayed with a binder solution (an aqueous solution of hydroxypropylmethyl cellulose) to prepare granules. After the granules were dried, they were coated by spraying a 5 wt % solution of hydroxypropylmethyl cellulose phthalate in a 1:1 mixture of ethanol and methylene chloride. The coated granules were mixed with magnesium stearate using a double cone mixer and compressed using a rotary compressor (MRC-33; Sejong) at a rate of 30 revolutions per minute (rpm) to provide tablets with a hardness of 7-9 kp, thickness of 3.0 mm and a diameter of 5.5 mm, which was used as core tablets.

2) Preparation of Rapid Release Simvastatin Layer

Predetermined amounts of simvastatin, microcrystalline cellulose and mannitol as shown in Table 2 were sieved with a No. 35 sieve and mixed using a high-speed mixer. A binder solution was prepared by dissolving hydroxypropyl cellulose and citric acid in water, and combining with the mixture of the main ingredients in a high-speed mixer. Thus obtained mixture was granulated using an oscillator with a No. 20 sieve, dried at 60° C. using a steam dryer and ground with a No. 20 sieve. The granules were mixed with butylatedhydroxyanisole using a double cone mixer.

3) Compression and Coating

Compression was performed with a compressor for the production of an inner core tablet (KUD-1: Kilian) at a rate of 30 revolutions per minute (rpm) using the amlodipine core tablet and the composition comprising simvastatin as an inner core and an outer layer, respectively, to provide a tablet with a hardness of 7-9 kp, a thickness of 6.0 mm and a diameter of 9.5 mm. Hi-coater (SFC-30N, Sejong mechanics, Korea), thus producing inner core tablets.

### Example 8

### Preparation of Amlodipine-Simvastatin Press Coated Tablets

1) Preparation of Delayed Release Amlodipine Core Tablets

Predetermined amounts of amlodipine and microcrystalline cellulose as shown in Table 2 were sieved with a No. 35 sieve, and mixed using a double cone mixer. The mixture was introduced into a high-speed mixer, combined with Kollicoat SR30D and granulated using an oscillator with a No. 20 sieve. After the granules were dried, they were ground with a No. 20 sieve. The sized granules were mixed with magnesium stearate using a double cone mixer and compressed using a rotary compressor (MRC-33; Sejong) at a rate of 30 revolutions per minute (rpm) to provide tablets with a hardness of 7-9 kp, thickness of 3.0 mm and a diameter of 5.5 mm, which was used as core tablets.

2) Preparation of Rapid Release Simvastatin Layer

Predetermined amounts of simvastatin, microcrystalline cellulose and mannitol as shown in Table 2 were sieved with a No. 35 sieve and mixed using a high-speed mixer. A binder solution was prepared by dissolving hydroxypropyl cellulose and citric acid in water, and combining with the mixture of the main ingredients in a high-speed mixer. Thus obtained mixture was granulated using an oscillator with a No. 20 sieve, dried at 60° C. using a steam dryer and ground with a No. 20 sieve. The granules were mixed with butylatedhydroxyanisole using a double cone mixer.

3) Compression and Coating

Compression was performed with a compressor for the production of an inner core tablet (KUD-1: Kilian) at a rate of 30 revolutions per minute (rpm) using the amlodipine core tablet and the composition comprising simvastatin as an inner core and an outer layer, respectively, to provide a tablet with a hardness of 7-9 kp, a thickness of 6.0 mm and a diameter of 9.5 mm. Hi-coater (SFC-30N, Sejong mechanics, Korea), thus producing inner core tablets.

### Example 9

### Preparation of Amlodipine-Simvastatin Biphasic Capsules

1) Preparation of Amlodipine Delayed Release Granules

Predetermined amounts of amlodipine and microcrystalline cellulose as shown in Table 2 are sieved using a No. 35 sieve, mixed using a double cone mixer. The mixture was introduced into a fluidized-bed granulator (GPCG 1: Glatt), granulated by spraying a binder solution (an aqueous solution of hydroxypropylmethyl cellulose) and dried. The granules were coated by spraying a 5 wt % solution prepared by dissolving hydroxypropylmethyl cellulose phthalate in a 11:1 mixture of ethanol and methylene chloride.

2) Preparation of Simvastatin Granules

Predetermined amounts of simvastatin, microcrystalline cellulose and mannitol as shown in Table 2 were sieved with a No. 35 sieve and mixed and mixed using a high-speed mixer. A binder solution prepared by dissolving hydroxypropyl cellulose and citric acid in water was combined with a mixture of main ingredients in a high-speed mixer and granulated using an oscillator with a No. 20 sieve. The granules were dried at 60° C. using a steam dryer and ground with a No. 20 sieve. The sized granules was added with butylatedhydroxyanisole and finally mixed using a double cone mixer.

3) Compression and Coating

The resulting composition prepared in the aforementioned processes 1) and 2) was mixed using a double cone mixer and added with sodium starch glycolate. The mixture was mixed using a double cone mixer, further mixed with colloidal silicon dioxide and finally mixed with magnesium stearate. The resulting mixture was introduced into a powder inlet and filled using a capsule filling machine.

### Example 10

### Preparation of Amlodipine-Simvastatin Biphasic Capsules

1) Preparation of Amlodipine Controlled-Release Granules

Predetermined amounts of amlodipine and microcrystalline cellulose as shown in Table 2 were sieved using a No. 35 sieve and mixed using a double cone mixer. The mixture was introduced into a fluidized-bed granulator (GPCG 1: Glatt), granulated by spraying Kollicoat SR30D and dried.

2) Preparation of Rapid Release Simvastatin Granules

Predetermined amounts of simvastatin, microcrystalline cellulose and mannitol as shown in Table 2 were sieved using a No. 35 sieve and mixed using a high-speed mixer. A binder solution prepared by dissolving hydroxypropyl cellulose and citric acid in water was combined with a mixture of main ingredients in a high-speed mixer and granulated using an oscillator with a No. 20 sieve. The granules were dried at 60° C. using a steam dryer and ground with a No. 20 sieve. The sized granules were finally mixed with butylatedhydroxyanisole.

3) Compression and Coating

The resulting composition prepared in the aforementioned processes 1) and 2) was mixed using a double cone mixer and added with sodium starch glycolate. The mixture was mixed using a double cone mixer, further mixed with colloidal silicon dioxide using a double cone mixer and finally mixed with magnesium stearate. The resulting mixture was introduced into a powder inlet and filled using a capsule filling machine.

TABLE 2

| | | Amounts (mg/tablet) | | | | | | | | | |
|---|---|---|---|---|---|---|---|---|---|---|---|
| | | Examples | | | | | | | | | |
| Ingredients | | 1 | 2 | 3 | 4 | 5 | 6 | 7 | 8 | 9 | 10 |
| Controlled- | Amlodipine maleate | 6.42 | 6.42 | 6.42 | 6.42 | 6.42 | 6.42 | 6.42 | 6.42 | 6.42 | 6.42 |
| release | Lercanidipine HCl | — | — | — | — | — | — | — | — | — | — |
| layer | Lacidipine | — | — | — | — | — | — | — | — | — | — |
| | Microcrystalline | 70.83 | 73.58 | 80.83 | 72.83 | 81.58 | 72.83 | 76.58 | 73.58 | 71.58 | 70.83 |
| | cellulose | | | | | | | | | | |
| | Kollicoat SR30D¹⁾ | — | 20 | — | 20 | — | 20 | — | 20 | — | — |
| | Eudragit RS PO²⁾ | — | — | — | — | — | — | — | — | 20 | 20 |
| | Carbomer 71G³⁾ | 10 | — | — | — | — | — | — | — | — | — |
| | Hydroxypropylmethyl | 2 | — | 2 | — | 2 | — | 7 | — | 2 | 2 |
| | cellulose | | | | | | | | | | |
| | Hydroxypropylmethyl | 10 | — | 10 | — | 10 | — | 10 | — | — | — |
| | cellulose phthalate | | | | | | | | | | |
| | Magnesium stearate | 0.75 | — | 0.75 | 0.75 | — | 0.75 | — | — | — | 0.75 |
| Immediate- | Simvastatin | 20 | 20 | 20 | 20 | 20 | 20 | 20 | 20 | 20 | 20 |
| release | Lovastatin | — | — | — | — | — | — | — | — | — | — |
| layer | Atorvastatin | — | — | — | — | — | — | — | — | — | — |
| | Microcrystalline | 57 | 57 | 57 | 57 | 57 | 57 | 57 | 57 | 57 | 57 |
| | cellulose | | | | | | | | | | |
| | D-mannitol | 112.46 | 112.46 | 112.46 | 112.46 | 112.46 | 112.46 | 112.46 | 112.46 | 112.46 | 112.46 |
| | Sodium starch glycolate | 1 | 1 | 1 | 1 | 1 | 1 | 1 | 1 | 1 | 1 |
| | Butylatedhydroxy- | 0.04 | 0.04 | 0.04 | 0.04 | 0.04 | 0.04 | 0.04 | 0.04 | 0.04 | 0.04 |
| | anisole | | | | | | | | | | |
| | Hydroxypropylmethyl | 5 | 5 | 5 | 5 | 5 | 5 | 5 | 5 | 5 | 5 |
| | cellulose | | | | | | | | | | |
| | Aerosil 200⁴⁾ | 1 | 1 | 1 | 1 | 1 | 1 | 1 | 1 | 1 | 1 |
| | Citric acid | 2 | 2 | 2 | 2 | 2 | 2 | 2 | 2 | 2 | 2 |
| | Magnesium stearate | 1.5 | 1.5 | 1.5 | 1.5 | 1.5 | 1.5 | 1.5 | 1.5 | 1.5 | 1.5 |
| Coating | Hydroxypropylmethyl | 2.6 | 2.6 | 2.6 | 2.6 | 2.6 | 2.6 | — | — | 2.6 | 2.6 |
| layer | cellulose 2910 | | | | | | | | | | |
| | Hydroxypropyl cellulose | 2.6 | 2.6 | 2.6 | 2.6 | 2.6 | 2.6 | — | — | 2.6 | 2.6 |
| | Titanium oxide | 2.3 | 2.3 | 2.3 | 2.3 | 2.3 | 2.3 | — | — | 2.3 | 2.3 |
| | Talc | 1.5 | 1.5 | 1.5 | 1.5 | 1.5 | 1.5 | — | — | 1.5 | 1.5 |
| | Ethanol | 64.8 | 64.8 | 64.8 | 64.8 | 64.8 | 64.8 | — | — | 64.8 | 64.8 |
| | Distilled water | 16.2 | 16.2 | 16.2 | 16.2 | 16.2 | 16.2 | — | — | 16.2 | 16.2 |

| | | | | | | | | | | |
|---|---|---|---|---|---|---|---|---|---|---|
| Total | 390 | 390 | 390 | 390 | 390 | 390 | 300 | 300 | 390 | 390 |

¹⁾Kollicoat SR30D-Main ingredient: polyacetate 30% suspension (BASF)
²⁾Eudragit RS PO-Main ingredient: polymethacrylate copolymer (BASF)
³⁾Carbomer 71G-Main ingredient: Carboxyvinylpolymer (Lubrizol)
⁴⁾Aerosil 200-Main ingredient: colloidal silicon dioxide (Degussa)

### Example 11

### Preparation of Amlodipine-Lovastatin Multi-Layered Tablets

1) Preparation of Amlodipine Controlled-Release Layer

Predetermined amounts of amlodipine maleate and microcrystalline cellulose as shown in Table 3 were sieved with a No. 35 sieve and mixed using a double cone mixer. The mixture was introduced into a fluidized-bed granulator (GPCG 1: Glatt), granulated by spraying a binder solution (an aqueous solution of hydroxypropylmethyl cellulose) and dried. The granules were coated by spraying a 5 wt % solution prepared by dissolving hydroxypropylmethyl cellulose phthalate in a 1:1 mixture of ethanol and methylene chloride, and finally mixed with magnesium stearate using a double cone mixer.

2) Preparation of Lovastatin Rapid Release Layer

Predetermined amounts of lovastatin, microcrystalline cellulose and mannitol as shown in Table 3 were sieved with a No. 35 sieve and mixed using a high-speed mixer. A binder solution prepared by dissolving hydroxypropyl cellulose and citric acid in water was combined with a mixture of main ingredients in a high-speed mixer and granulated using an oscillator with a No. 20 sieve. The granules were dried at 60° C. using a steam dryer and ground with a No. 20 sieve. The sized granules were mixed with butylatedhydroxyanisole, sodium starch glycolate and colloidal silicon dioxide and finally mixed with magnesium stearate using a double cone mixer.

3) Compression and Coating

Compression was performed using a compressor for the production of multi-layered tablet (MRC-37T: Sejong). In detail, the composition comprising simvastatin was input in a first powder inlet and the composition comprising amlodipine was input in a second inlet. The compression was performed under such a condition that the interlayer incorporation may be minimized at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea), thus producing multi-layered tablets.

### Example 12

### Preparation of Amlodipine-Lovastatin Multi-Layered Tablets

1) Preparation of Amlodipine Controlled-Release Layer

Predetermined amounts of amlodipine maleate and microcrystalline cellulose as shown in Table 3 were sieved with a No. 35 sieve and mixed using a double cone mixer. The mixture was introduced into a high-speed mixer, combined by adding Kollicoat SR30D and granulated using an oscillator with a No. 20 sieve. The granules were dried at 60° C. using a steam dryer and tabulated with a No. 20 sieve. The sized granules were finally mixed with magnesium stearate using a double cone mixer.

2) Preparation of Lovastatin Rapid Release Layer

Predetermined amounts of lovastatin, microcrystalline cellulose and mannitol as shown in Table 3 were sieved with a No. 35 sieve and mixed using a high-speed mixer. A binder solution prepared by dissolving hydroxypropyl cellulose and citric acid in water was combined with a mixture of main ingredients in a high-speed mixer and granulated using an oscillator with a No. 20 sieve. The granules were dried at 60° C. using a steam dryer and ground with a No. 20 sieve. The sized granules were mixed with butylatedhydroxyanisole, sodium starch glycolate and colloidal silicon dioxide and finally mixed with magnesium stearate using a double cone mixer.

3) Compression and Coating

Compression was performed using a compressor for the production of multi-layered tablet (MRC-37T: Sejong). In detail, the composition comprising lovastatin was input in a first power inlet and the composition comprising amlodipine was input in a second inlet. The compression was performed under such a condition that the interlayer incorporation may be minimized at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea), thus producing multi-layered tablets.

### Example 13

### Preparation of Amlodipine-Atorvastatin Multi-Layered Tablets

1) Preparation of Amlodipine Controlled-Release Layer

Predetermined amounts of amlodipine and microcrystalline cellulose as shown in Table 3 were sieved with a No. 35 sieve and mixed using a double cone mixer. The mixture Was introduced into a fluidized-bed granulator (GPCG 1: Glatt), granulated by spraying a binder solution (an aqueous solution of hydroxypropylmethyl cellulose) and dried. The granules were coated by spraying a 5 wt % solution prepared by dissolving hydroxypropylmethyl cellulose phthalate in a 1:1 mixture of ethanol and methylene chloride. The coated granules were finally mixed with magnesium stearate using a double cone mixer.

2) Preparation of Atorvastatin Layer

Predetermined amounts of atorvastatin calcium, microcrystalline cellulose and mannitol as shown in Table 3 were sieved with a No. 35 sieve and mixed using a high-speed mixer. A binder solution prepared by dissolving hydroxypropyl cellulose and citric acid in water was combined with a mixture of main ingredients in a high-speed mixer and granulated using an oscillator with a No. 20 sieve. The granules were dried at 60° C. using a steam dryer and ground with a No. 20 sieve. The sized granules were mixed with butylatedhydroxyanisole, sodium starch glycolate and colloidal silicon dioxide and finally mixed with magnesium stearate using a double cone mixer.

3) Compression and Coating

Compression was performed using a compressor for the production of multi-layered tablet (MRC-37T: Sejong). In detail, the composition comprising atorvastatin was input in a first power inlet and the composition comprising amlodipine was input in a second inlet. The compression was performed under such a condition that the interlayer incorporation may be minimized at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea), thus producing multi-layered tablets.

### Example 14

### Preparation of Amlodipine-Atorvastatin Multi-Layered Tablets

1) Preparation of Amlodipine Controlled-Release Layer

Predetermined amounts of amlodipine and microcrystalline cellulose as shown in Table 3 were sieved with a No. 35 sieve and mixed using a double cone mixer. The mixture was introduced into a high-speed mixer, combined by adding Kollicoat SR30D and granulated using an oscillator with a No. 20 sieve. The granules were dried at 60° C. using a steam dryer and ground with a No. 20 sieve. The sized granules were mixed with magnesium stearate using a double cone mixer.

2) Preparation of Atorvastatin Layer

Predetermined amounts of atorvastatin, microcrystalline cellulose and mannitol as shown in Table 3 were sieved with a No. 35 sieve and mixed using a high-speed mixer. A binder solution prepared by dissolving hydroxypropyl cellulose and citric acid in water was combined with a mixture of main ingredients in a high-speed mixer and granulated using an oscillator with a No. 20 sieve. The granules were dried at 60° C. using a steam dryer and ground with a No. 20 sieve. The sized granules were mixed with butylatedhydroxyanisole, sodium starch glycolate and colloidal silicon dioxide and finally mixed with magnesium stearate using a double cone mixer.

3) Compression and Coating

Compression was performed using a compressor for the production of multi-layered tablet (MRC-37T: Sejong). In detail, the composition comprising atorvastatin was input in a first power inlet and the composition comprising amlodipine was input in a second inlet. The compression was performed under such a condition that the interlayer incorporation may be minimized at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea), thus producing multi-layered controlled-release tablets.

### Example 15

### Preparation of Amlodipine-Atorvastatin Press-Coated Tablets

1) Preparation of Amlodipine Core Tablet

Predetermined amounts of amlodipine and microcrystalline cellulose as shown in Table 3 were sieved with a No. 35 sieve and mixed using a double cone mixer, The mixture were introduced into a fluidized-bed granulator (GPCG 1: Glatt), granulated by spraying a binder solution (an aqueous solution of hydroxypropylmethyl cellulose) and dried. The granules were added with carbomer 71G powders, and mixed with magnesium stearate with a double cone mixer. The resulting mixture was compressed using a rotary compressor (MRC-33: Sejong) at a speed of 30 revolutions per minute (rpm) to provide tablets with a hardness of 7-9 kp, a thickness of 3.0 mm and a diameter of 5.5 mm. These tablets were used as core tablets.

2) Preparation of Atorvastatin Layer

Predetermined amounts of atorvastatin, microcrystalline cellulose and mannitol as shown in Table 3 were sieved with a No. 35 sieve and mixed using a high-speed mixer. A binder solution prepared by dissolving hydroxypropyl cellulose and citric acid in water was combined with a mixture of main ingredients in a high-speed mixer and granulated using an oscillator with a No. 20 sieve. The granules were dried at 60° C. using a steam dryer and ground with a No. 20 sieve. The sized granules were mixed with butylatedhydroxyanisole, sodium starch glycolate and colloidal silicon dioxide and finally mixed with magnesium stearate using a double cone mixer.

3) Compression and Coating

Press coated tablets were prepared with a compressor for the production of press-coated tablets (RUD-1: Kilian) by using the amlodipine core tablet and the composition containing atorvastatin as an inner core and an outer layer, respectively. The compression was performed at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea).

### Example 16

### Preparation of Lercanidipine-Simvastatin Multi-Layered Tablets

1) Preparation of Lercanidipine Controlled-Release Layer

Predetermined amounts of lercanidipine HCl and microcrystalline cellulose as shown in Table 3 were sieved with a No. 35 sieve and mixed using a double cone mixer. The mixture was placed into a fluidized-bed granulator (GPCG 1: Glatt), and sprayed with a binder solution (an aqueous solution of hydroxypropylmethyl cellulose) to prepare granules, and dried. The granules were coated by spraying a 5 wt % solution prepared by dissolving hydroxypropylmethyl cellulose phthalate in a 1:1 mixture of ethanol and methylene chloride. The coated granules were finally mixed with magnesium stearate using a double cone mixer.

2) Preparation of Rapid Release Simvastatin Layer

Predetermined amounts of simvastatin, microcrystalline cellulose and mannitol as shown in Table 3 were sieved with a No. 35 sieve and mixed using a high-speed mixer. A binder solution prepared by dissolving hydroxypropyl cellulose and citric acid in water was combined with a mixture of main ingredients in a high-speed mixer and granulated using an oscillator with a No. 20 sieve. The granules were dried at 60° C. using a steam dryer and ground with a No. 20 sieve. The sized granules were mixed with butylatedhydroxyanisole, sodium starch glycolate and colloidal silicon dioxide and finally mixed with magnesium stearate using a double cone mixer.

3) Compression and Coating

Compression was performed using a compressor for the production of multi-layered tablet (MRC-37T: Sejong). In detail, the composition comprising simvastatin was introduced into a first powder inlet, and then the composition comprising lercanidipine was introduced into a second powder inlet. The compression was performed under such a condition that the interlayer incorporation may be minimized at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea), thus producing multi-layered tablets.

### Example 17

### Preparation of Lercanidinine-Simvastatin Multi-Layered Tablets

1) Preparation of Lercanidipine Controlled-Release Layer

Predetermined amounts of lercanidipine and microcrystalline cellulose as shown in Table 3 were sieved with a No. 35 sieve and mixed using a double cone mixer. The mixture was introduced into a high-speed mixer, combined by adding Kollicoat SR30D and granulated using an oscillator with a No. 20 sieve. The granules were dried at 60° C. using a steam dryer and ground with a No. 20 sieve. The sized granules were mixed with magnesium stearate using a double cone mixer.

2) Preparation of Simvastatin Rapid Release Layer

Predetermined amounts of simvastatin, microcrystalline cellulose and mannitol as shown in Table 3 were sieved with a No. 35 sieve and mixed using a high-speed mixer. A binder solution prepared by dissolving hydroxypropyl cellulose and citric acid in water was combined with a mixture of main ingredients in a high-speed mixer and granulated using an oscillator with a No. 20 sieve. The granules were dried at 60° C. using a steam dryer and ground with a No. 20 sieve. The sized granules were mixed with butylatedhydroxyanisole, sodium starch glycolate and colloidal silicon dioxide, and finally mixed with magnesium stearate using a double cone mixer.

3) Compression and Coating

Compression was performed using a compressor for the production of multi-layered tablet (MRC-37T: Sejong). In detail, the composition comprising simvastatin was introduced into a first powder inlet, and the composition comprising lacidipine was introduced into a second powder inlet. The compression was performed under such a condition that the interlayer incorporation may be minimized at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea), thus producing multi-layered tablets.

### Example 18

### Preparation of Lacidipine-Simvastatin Multi-Layered Tablets

1) Preparation of Lacidipine Controlled-Release Layer

Predetermined amounts of lacidipine and microcrystalline cellulose as shown in Table 3 were sieved with a No. 35 sieve and mixed using a double cone mixer. The mixture was introduced into a fluidized-bed granulator (GPCG 1: Glatt), granulated by spraying a binder solution (an aqueous solution of hydroxypropylmethyl cellulose) and dried. The granules were coated by spraying a 5 wt % solution prepared by dissolving hydroxypropylmethyl cellulose phthalate in a 1:1 mixture of ethanol and methylene chloride, and finally mixed with magnesium stearate using a double cone mixer.

2) Preparation of Simvastatin Rapid Release Layer

Predetermined amounts of simvastatin, microcrystalline cellulose and mannitol as shown in Table 3 were sieved with a No. 35 sieve and mixed using a high-speed mixer. A binder solution prepared by dissolving hydroxypropyl cellulose and citric acid in water was combined with a mixture of main ingredients in a high-speed mixer and granulated using an oscillator with a No. 20 sieve. The granules were dried at 60° C. using a steam dryer and ground with a No. 20 sieve. The sized granules were mixed with butylatedhydroxyanisole, sodium starch glycolate and colloidal silicon dioxide, and finally mixed with magnesium stearate using a double cone mixer.

3) Compression and Coating

Compression was performed using a compressor for the production of multi-layered tablet (MRC-37T: Sejong). In detail, the composition comprising simvastatin was introduced into a first powder inlet, and the composition comprising lacidipine was introduced into a second powder inlet. The compression was performed under such a condition that the interlayer incorporation may be minimized at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea), thus producing multi-layered tablets.

### Example 19

### Preparation of Lacidipine-Simvastatin Multi-Layered Tablets

1) Preparation of Lacidipine Controlled-Release Layer Predetermined amounts of lacidipine and microcrystalline cellulose as shown in Table 3 were sieved with a No. 35 sieve, and mixed using a double cone mixer. The mixture was mixed using Kollicoat SR30D in a high-speed mixer. Thus obtained mixture was granulated using oscillator with a No. 20 sieve, dried at 60° C. using a steam dryer and sized with a No. 20 sieve. The coated granules were mixed with magnesium stearate using a double cone mixer.

2) Preparation of Simvastatin Layer

Predetermined amounts of simvastatin, microcrystalline cellulose and mannitol as shown in Table 3 were sieved with a No. 35 sieve and mixed using a high-speed mixer. A binder solution was prepared by dissolving hydroxypropyl cellulose and citric acid in water and combining with the mixture of the main ingredients. Thus obtained mixture was combined, granulated using an oscillator with a No. 20 sieve, dried at 60° C. using a steam dryer and ground with a No. 20 sieve. The granules were mixed with butylatedhydroxyanisole, sodium starch glycolate and colloidal silicon dioxide, and finally mixed with magnesium stearate using a double cone mixer.

3) Compression and Coating

The composition was compressed using a compressor for the production of a multi-layered tablet (MRC-37: Sejong). In detail, the composition comprising simvastatin was input in a first power inlet and the composition comprising amlodipine was input in a second inlet. The compression was performed under such a condition that the interlayer incorporation may be minimized at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea), thus producing multi-layered tablets.

TABLE 3

| | | Amounts (mg/tablet) | | | | | | | | |
|---|---|---|---|---|---|---|---|---|---|---|
| | | Examples | | | | | | | | |
| Ingredients | | 11 | 12 | 13 | 14 | 15 | 16 | 17 | 18 | 19 |
| Controlled- | Amlodipine maleate | 6.42 | 6.42 | 6.42 | 6.42 | 6.42 | — | — | — | — |
| release | Lercanidipine HCl | — | — | — | — | — | 10 | 10 | — | — |
| layer | Lacidipine | — | — | — | — | — | — | — | 4 | 4 |
| | Microcrystalline | 80.83 | 72.83 | 80.83 | 72.83 | 70.83 | 79.25 | 64.25 | 78.25 | 85.25 |
| | cellulose | | | | | | | | | |
| | Kollicoat SR30D¹⁾ | — | 20 | — | 20 | — | — | 25 | — | 20 |
| | Eudragit RS PO²⁾ | — | — | — | — | — | — | — | — | — |
| | Carbomer 71G³⁾ | — | — | — | — | 10 | — | — | — | — |
| | Hydroxypropylmethyl | 2 | — | 2 | — | 2 | 4 | — | 7 | — |
| | cellulose | | | | | | | | | |
| | Hydroxypropylmethyl | 10 | — | 10 | — | 3 | 6 | — | 10 | — |
| | cellulose phthalate | | | | | | | | | |
| | Magnesium stearate | 0.75 | 0.75 | 0.75 | 0.75 | 0.75 | 0.75 | 0.75 | 0.75 | 0.75 |
| Immediate- | Simvastatin | — | — | — | — | — | 20 | 20 | 20 | 20 |
| release | Lovastatin | 20 | 20 | — | — | — | — | — | — | — |
| layer | Atorvastatin | — | — | 20 | 20 | 20 | — | — | — | — |
| | Microcrystalline | 57 | 57 | 57 | 57 | 57 | 57 | 57 | 57 | 57 |
| | cellulose | | | | | | | | | |
| | D-mannitol | 112.46 | 112.46 | 112.46 | 112.46 | 112.46 | 112.46 | 112.46 | 112.46 | 112.46 |
| | Sodium starch glycolate | 1 | 1 | 1 | 1 | 1 | 1 | 1 | 1 | 1 |
| | Butylatedhydroxy- | 0.04 | 0.04 | 0.04 | 0.04 | 0.04 | 0.04 | 0.04 | 0.04 | 0.04 |
| | anisole | | | | | | | | | |
| | Hydroxypropylmethyl | 5 | 5 | 5 | 5 | 5 | 5 | 5 | 5 | 5 |
| | cellulose | | | | | | | | | |
| | Aerosil 200⁴⁾ | 1 | 1 | 1 | 1 | 1 | 1 | 1 | 1 | 1 |
| | Citric acid | 2 | 2 | 2 | 2 | 2 | 2 | 2 | 2 | 2 |
| | Magnesium stearate | 1.5 | 1.5 | 1.5 | 1.5 | 1.5 | 1.5 | 1.5 | 1.5 | 1.5 |
| Coating | Hydroxymethyl cellulose | 2.6 | 2.6 | 2.6 | 2.6 | 2.6 | 2.6 | 2.6 | 2.6 | 2.6 |
| layer | 2910 | | | | | | | | | |
| | Hydroxypropyl cellulose | 2.6 | 2.6 | 2.6 | 2.6 | 2.6 | 2.6 | 2.6 | 2.6 | 2.6 |
| | Titanium oxide | 2.3 | 2.3 | 2.3 | 2.3 | 2.3 | 2.3 | 2.3 | 2.3 | 2.3 |
| | Talc | 1.5 | 1.5 | 1.5 | 1.5 | 1.5 | 1.5 | 1.5 | 1.5 | 1.5 |
| | Ethanol | 64.8 | 64.8 | 64.8 | 64.8 | 64.8 | 64.8 | 64.8 | 64.8 | 64.8 |
| | Distilled water | 16.2 | 16.2 | 16.2 | 16.2 | 16.2 | 16.2 | 16.2 | 16.2 | 16.2 |

| | | | | | | | | | |
|---|---|---|---|---|---|---|---|---|---|
| Total | 390 | 390 | 390 | 390 | 383 | 390 | 300 | 300 | 390 |

¹⁾Kollicoat SR30D-Main ingredient: polyacetate 30% suspension (BASF)
²⁾Eudragit RS PO-Main ingredient: polymethacrylate copolymer (BASF)
³⁾Carbomer 71G-Main ingredient: Carboxyvinylpolymer (Lubrizol)
⁴⁾Aerosil 200-Main ingredient: colloidal silicon dioxide (Degussa)

### Example 20

### Preparation of Amlodipine-Simvastatin Press-Coated Tablets

1) Preparation of Amlodipine Timed-Release Layer (Inner Core)

Predetermined amounts of amlodipine besylate, microcrystalline cellulose and dicalcium phosphate as shown in Table 4 were sieved with a No. 35 sieve, and mixed using a double cone mixer. The mixture was placed into a fluidized-bed granulator (GPCG 1: Glatt), and sprayed with a binder solution (an aqueous solution of hydroxypropylmethyl cellulose) to prepare granules, and dried. The granules were mixed with carbomer 71G powders, and mixed with magnesium stearate with a double cone mixer. The resulting mixture was compressed using a rotary compressor (MRC-33: Sejong) at a speed of 30 revolutions per minute (rpm) to provide tablets with a hardness of 7-9 kp, a thickness of 3.0 mm and a diameter of 5.5 mm. After the tablets were compressed, a film coating layer was formed on the compressed tablets by spraying a solution of hydroxypropylmethyl cellulose phthalate and acetylated monoglyceride in a 1:1 mixture of ethanol and methylene chloride using Hi-coater (SFC-30N, Sejong mechanics, Korea), thereby producing core tablets.

2) Preparation of a Simvastatin Immediate-Release Layer

Predetermined amounts of simvastatin, microcrystalline cellulose, corn starch and lactose as shown in Table 4 were sieved with a No. 35 sieve and mixed using a high-speed mixer. A binder solution prepared by dissolving hydroxypropyl cellulose and citric acid in water, and combined with the mixture containing the main ingredients. Thus obtained mixture was granulated using an oscillator with a No. 20 sieve, dried at 60° C. using a steam dryer, ground with a No. 20 sieve. The resulting mixture was mixed with butylatedhydroxyanisole, sodium starch glycolate and colloidal silicon dioxide, and finally mixed with magnesium stearate using a double cone mixer.

3) Compression and Coating

Press-coated tablets were prepared with a compressor for the production of press-coated tablet (RUD-1: Kilian) by using the amlodipine core tablet and the composition containing simvastatin as an inner core and an outer layer, respectively. The compression was performed at a speed of 30 revolutions per minute (rpm) to provide tablets with a hardness of 7-9 kp, a thickness of 6.0 mm and a diameter of 9.5 mm. A film coating layer was formed on the compressed tablets using Hi-coater (SFC-30N, Sejong mechanics, Korea).

### Example 21

### Preparation of Amlodipine-Simvastatin Multi-Layered Tablets

1) Preparation of Amlodipine Timed-Release Layer

Predetermined amounts of amlodipine besylate, microcrystalline cellulose and dicalcium phosphate as shown in Table 4 were sieved with a No. 35 sieve, and mixed using a double cone mixer and sprayed with a binder solution (an aqueous solution of hydroxypropylmethyl cellulose) to prepare granules, and dried. After the granules were dried, they were coated by spraying a solution of hydroxypropylmethyl cellulose phthalate and acetylated monoglyceride in a 1:1 mixture of ethanol and methylene chloride. The coated granules were mixed with magnesium stearate using a double cone mixer.

2) Preparation of a Simvastatin Immediate-Release Layer

A simvastatin immediate-release layer was prepared by predetermined amounts of ingredients as shown in Table 3 and the method of preparing a simvastatin immediate-release layer of example 1.

3) Compression and Coating

Compression was performed using a compressor for the production of multi-layered tablet (MRC-37T: Sejong). In detail, the composition comprising simvastatin was introduced into a first powder inlet, and the composition comprising amlodipine was introduced into a second powder inlet. The compression was performed under such a condition that the interlayer incorporation may be minimized at a speed of 30 revolutions per minute (rpm) to provide tablets with a hardness of 7-9 kp, a thickness of 6.0 mm and a diameter of 9.5 mm. A film coating layer was formed on the compressed tablets using Hi-coater (SFC-30N, Sejong mechanics, Korea), thereby producing multi-layered timed-release tablets.

### Example 22

### Preparation of Capsules Comprising with Amlodipine Tablets-Simvastatin Tablets

1) Preparation of Amlodipine Timed-Release Layer (Tablet)

Predetermined amounts of amlodipine besylate and microcrystalline cellulose as shown in Table 4 were sieved with a No. 35 sieve, and mixed using a double cone mixer. The mixture was placed into a fluidized-bed granulator (GPCG 1: Glatt), and sprayed with a binder solution (an aqueous solution of hydroxypropylmethyl cellulose) to prepare granules, and dried. The granules were combined with carbomer 71G powders, and mixed with magnesium stearate with a double cone mixer. The resulting mixture was compressed using a rotary compressor (MRC-33: Sejong) at a speed of 30 revolutions per minute (rpm) to provide tablets with a hardness of 7-9 kp, a thickness of 3.0 mm and a diameter of 5.5 mm. After the tablets were compressed, film coating layer was formed on the compressed tablets by spraying a solution of hydroxypropylmethyl cellulose phthalate and acetylated monoglyceride in a 1:1 mixture of ethanol and methylene chloride using Hi-coater (SFC-30N, Sejong mechanics, Korea), thereby producing tablets.

2) Preparation of a Simvastatin Immediate-Release Layer

Predetermined amounts of simvastatin, microcrystalline cellulose and mannitol as shown in Table 4 were sieved with a No. 35 sieve and mixed using a high-speed mixer. A binder solution prepared by dissolving hydroxypropyl cellulose and citric acid in water, and combined with the mixture containing the main ingredients. Thus obtained mixture was granulated using an oscillator with a No. 20 sieve, dried at 60° C. using a steam dryer, ground with a No. 20 sieve. The resulting mixture was mixed with butylatedhydroxyanisole, sodium starch glycolate and colloidal silicon dioxide, and mixed with magnesium stearate with a double cone mixer. The resulting mixture was compressed using a rotary compressor (MRC-33: Sejong) at a speed of 30 revolutions per minute (rpm) to provide tablets with a hardness of 7-9 kp, a thickness of 4.0 mm and a diameter of 5.0 mm.

3) Capsule Filling

The amlodipine tablets prepared in the aforementioned processes 1) and the simvastatin tablets prepared in the aforementioned processes 2) were filled into No. 3 gelatin hard capsules using a capsule filling machine.

### Example 23

### Preparation of Amlodipine-Simvastatin Capsules

1) Preparation of Amlodipine Timed-Release Layer (Tablet)

Amlodipine timed-release layer was prepared by predetermined amounts of ingredients as shown in Table 4 and the method of preparing an amlodipine timed-release layer of Example 22.

2) Preparation of a Simvastatin Immediate-Release Layer (Granules)

Predetermined amounts of simvastatin, microcrystalline cellulose, corn starch and lactose as shown in Table 4 were sieved with a No. 35 sieve and mixed using a high-speed mixer. A binder solution prepared by dissolving hydroxypropyl cellulose and citric acid in water, and combined with the mixture containing the main ingredients. Thus obtained mixture was granulated using an oscillator with a No. 20 sieve, dried at 60° C. using a steam dryer, ground with a No. 20 sieve. The resulting mixture was mixed with butylatedhydroxyanisole, sodium starch glycolate and colloidal silicon dioxide, and finally mixed with magnesium stearate using a double cone mixer.

3) Capsule Filling

The amlodipine tablets prepared in the aforementioned processes 1) and the simvastatin granules prepared in the aforementioned processes 2) were filled into No. 2 hydroxypropylmethyl cellulose hardcapsules using a capsule filling machine.

### Example 24

### Preparation of Amlodipine-Simvastatin Coated Tablets

1) Preparation of Amlodipine Timed-Release Layer (Tablet)

Amlodipine timed-release layer was prepared by predetermined amounts of ingredients as shown in Table 4 and a method of preparing an amlodipine timed-release layer of example 20.

2) Preparation of a Simvastatin Immediate-Release Coating Solution

Predetermined amounts of simvastatin, butylatedhydroxyanisole, hydroxypropylmethylcellulose, colloidal silicon dioxide, polyethylene glycol 6000, titanium oxide and talc as shown in Table 4 were dissolved and dispersed in mixture of ethanol and methylene chloride to make a simvastatin immediate-release coating solution.

3) Primary Coating

After the aforemention amlodipine tablets were compressed, a primary coating layer was formed on the compressed tablets by spraying a simvastatin immediate-release coating solution using Hi-coater (SFC-30N, Sejong mechanics, Korea), thereby producing primary coated tablets.

4) Secondary Coating

After preparation of a secondary coating solution was provided by predetermined amounts of ingredients as shown in coating layer of Table 4, film coating layer was formed on the primary coated tablets by spraying a secondary coating solution, thereby producing film coated tablets.

### Example 25

### Preparation of Simvastatin Immediate-Release-Amlodipine Osmotic Press-Coated Tablets

1) Preparation of Amlodipine Timed-Release Layer (Tablets)

Predetermined amounts of amlodipine besylate, microcrystalline cellulose and dicalcium phosphate as shown in Table 4 were sieved with a No. 35 sieve, and mixed using a double cone mixer and mixed with magnesium stearate with a double cone mixer. The resulting mixture was compressed using a rotary compressor (MRC-33: Sejong) at a speed of 30 revolutions per minute (rpm) to provide tablets with a hardness of 7-9 kp, a thickness of 3.0 mm and a diameter of 5.5 mm. After the tablets were compressed, an osmotic coating layer was formed on the compressed tablets by spraying a solution of kollicoat SR30D and triethyl citrate, which are osmotic agents, in distilled water using Hi-coater (SFC-30N, Sejong mechanics, Korea), thereby producing osmotic core tablets.

2) Preparation of a Simvastatin Immediate-Release Layer

A simvastatin immediate-release layer was prepared by predetermined amounts of ingredients as shown in Table 4 and a method of preparing a simvastatin immediate-release layer of example 20.

3) Compression and Coating

Simvastatin immediate-release-amlodipine osmotic press-coated tablets were prepared by the method of compression and coating of example 20.

### Example 26

### Preparation of a Amlodipine-Simvastatin Blister Package Kits

Amlodipine-simvastatin blister package kits were prepared from predetermined amounts of ingredients as shown in Table 4 and the method of preparing an amlodipine timed-release layer of example 22, with the exception that instead of filling into capsules, the amlodipine and simvastatin tablets were packed in a blister package, with each blister containing an amlodipine tablet and a simvastatin tablet for co-administration.

### Example 27

### Preparation of Amlodipine-Atorvastatin Press-Coated Tablets

Amlodipine-atorvastatin press-coated tablets were prepared from predetermined amounts of ingredients as shown in Table 4 and the method of preparing an amlodipine timed-release layer of example 20, with the exception that atorvastatin was used as statin instead of simvastatin.

### Example 28

### Preparation of Amlodipine-Atorvastatin Multi-Layered Tablets

Amlodipine-atorvastatin multi-layered tablets were prepared from predetermined amounts of ingredients as shown in Table 4 and the method of preparing an amlodipine timed-release layer of example 21, with the exception that atorvastatin was used as statin instead of simvastatin.

### Example 29

### Preparation of Amlodipine-Atorvastatin Capsules

An amlodipine-atorvastatin capsule was prepared from predetermined amounts of ingredients as shown in Table 4 and the method of preparing an amlodipine timed-release layer of Example 22, with the exception that atorvastatin was used as statin instead of simvastatin.

TABLE 4

| | | Amounts (mg/tablet) | | | | | | | | | |
|---|---|---|---|---|---|---|---|---|---|---|---|
| | | Examples | | | | | | | | | |
| Ingredients | | 20 | 21 | 22 | 23 | 24 | 25 | 26 | 27 | 28 | 29 |
| Timed- | Amlodipine besylate | 6.94 | 6.94 | 6.94 | 6.94 | 6.94 | 6.94 | 6.94 | 6.94 | 6.94 | 6.94 |
| release | Microcrystalline | 40.46 | 50.46 | 57.46 | 57.46 | 127.46 | 62.06 | 57.46 | 40.46 | 50.46 | 57.46 |
| layer | cellulose | | | | | | | | | | |
| | Dicalcium phosphate | 16 | 15 | — | — | 30 | — | — | 16 | 15 | — |
| | Sodium chloride | — | — | — | — | — | 10 | — | — | — | — |
| | Hydroxypropylmethyl | 4 | 4 | 3 | 3 | 6 | — | 3 | 4 | 4 | 3 |
| | cellulose | | | | | | | | | | |
| | Carbomer 71G¹⁾ | 10 | — | 10 | 10 | 15 | — | 10 | 10 | — | 10 |
| | Hydroxypropylmethyl | 10 | 20 | 10 | 10 | 12 | — | 10 | 10 | 20 | 10 |
| | cellulose phthalate | | | | | | | | | | |
| | Kollicoat SR30D | — | — | — | — | — | 12 | — | — | — | — |
| | Acetylated | 2 | 3 | 2 | 2 | 2 | — | 2 | 2 | 3 | 2 |
| | monoglyceride | | | | | | | | | | |
| | Triethyl citrate | — | — | — | — | — | 2 | — | — | — | — |
| | Magnesium stearate | 0.6 | 0.6 | 0.6 | 0.6 | 1 | 1 | 0.6 | 0.6 | 0.6 | 0.6 |
| Immediate- | Simvastatin | 20 | 20 | 20 | 20 | 20 | 20 | 20 | | | |
| release | Atorvastatin | — | — | — | — | — | — | — | 10.85 | 10.85 | 21.69 |
| layer | Microcrystalline | 80 | 48.96 | 10.85 | 20 | — | 80 | 10.85 | 85.15 | 48.11 | 19.12 |
| | cellulose | | | | | | | | | | |
| | D-mannitol | — | 120 | 53.61 | — | — | — | 53.61 | — | 120 | 53.61 |
| | Lactose | 160.46 | — | — | 40.46 | — | 160.46 | — | 160.46 | — | — |
| | Corn Starch | 20 | — | — | 10 | — | 20 | — | 20 | — | — |
| | Sodium starch glycolate | 2 | 2 | 2 | 2 | — | 2 | 2 | 2 | 2 | 2 |
| | Butylated hydroxyanisole | 0.04 | 0.04 | 0.04 | 0.04 | 0.05 | 0.04 | 0.04 | 0.04 | 0.04 | 0.08 |
| | Hydroxypropyl-cellulose | 6 | 5 | 3 | 3 | — | 6 | 3 | 6 | 5 | 3 |
| | Colloidal Silicon | 2 | 1 | 2 | 2 | 2.95 | 2 | 2 | 2 | 1 | 2 |
| | dioxide²⁾ | | | | | | | | | | |
| | Citric acid | 2 | 2 | 2 | 2 | — | 2 | 2 | 2 | 2 | 2 |
| | Magnesium stearate | 1.5 | 1 | 0.5 | 0.5 | — | 1.5 | 0.5 | 1.5 | 1 | 0.5 |
| | Hydroxypropylmethyl | — | — | 4 | — | 15 | — | 4 | — | — | 4 |
| | cellulose 2910 | | | | | | | | | | |
| | Polyethylene glycol 6000 | — | — | 1 | — | 2 | — | 1 | — | — | 1 |
| | Titanium oxide | — | — | 0.8 | — | 1 | — | 0.8 | — | — | 0.8 |
| | Talc | — | — | 0.2 | — | 1 | — | 0.2 | — | — | 0.2 |
| Coating | Hydroxypropylmethyl | 6 | 5 | — | — | 3 | 6 | — | 6 | 5 | — |
| layer | cellulose 2910 | | | | | | | | | | |
| | Hydroxypropyl cellulose | 6 | 5 | — | — | 3 | 6 | — | 6 | 5 | — |
| | Titanium oxide | 2.5 | 2 | — | — | 1 | 2.5 | — | 2.5 | 2.5 | — |
| | Talc | 1.5 | 1 | — | — | 1 | 1.5 | — | 1.5 | 1.5 | — |
| | Ethanol | (80) | (75) | — | — | (48) | (80) | | (80) | (75) | — |
| | Purified water | (20) | (15) | — | — | (12) | (20) | | (20) | (15) | — |

| | | | | | | | | | | |
|---|---|---|---|---|---|---|---|---|---|---|
| Empty Capsule | — | — | 50 | 65 | — | — | — | — | — | 50 |
| Total | 400 | 313 | 240 | 255 | 250 | 404 | 190 | 396 | 304 | 250 |

¹⁾Carbomer 71G-Main ingredient: Carboxyvinylpolymer (Lubrizol)
²⁾Colloidal silicon dioxide-Commercial name: Aerosil 200 (Degussa)

### Example 30

### Preparation of Amlodipine-Atorvastatin Capsules

1) Preparation of Amlodipine Timed-Release Layer (Tablet)

An amlodipine timed-release layer was prepared from predetermined amounts of ingredients as shown in Table 4 and the method of preparing an amlodipine timed-release layer of example 22.

2) Preparation of an Atorvastatin Immediate-Release Layer (Granule)

An atorvastatin immediate-release layer was prepared from predetermined amounts of ingredients as shown in Table 4 and the method of preparing an atorvastatin immediate-release layer of example 23.

3) Capsule Filling

The amlodipine tablets prepared in the aforementioned processes 1) and the atorvastatin granules prepared in the aforementioned processes 2) were filled in hydroxypropylmethyl cellulose hard capsules using a capsule filling machine.

### Example 31

### Preparation of Amlodipine-Atorvastatin Coated Tablets

Amlodipine-atorvastatin coating tablets were prepared from predetermined amounts of ingredients as shown in Table 5 and the method of example 24, with the exception that atorvastatin was used as statin instead of simvastatin.

### Example 32

### Preparation of Amlodipine-Atorvastatin Osmotic Press-Coated Tablets

1) Preparation of Amlodipine Timed-Release Layer (Inner Core)

Predetermined amounts of amlodipine besylate and microcrystalline cellulose, sodium chloride as shown in Table 5 were sieved with a No. 35 sieve, and mixed with magnesium stearate with a double cone mixer. The resulting mixture was compressed using a rotary compressor (MRC-33: Sejong) at a speed of 30 revolutions per minute (rpm) to provide tablets with a hardness of 7-9 kp, a thickness of 3.0 mm and a diameter of 5.5 mm. After the tablets were compressed, osmotic coating layer was formed on the compressed tablets by spraying a solution of Eudragit RS 30D and triethyl citrate, which are osmotic agents, in distilled water using Hi-coater (SFC-30N, Sejong mechanics, Korea), thereby producing osmotic core tablets.

2) Preparation of a Simvastatin Immediate-Release Layer

A simvastatin immediate-release layer was provided from predetermined amounts of ingredients as shown in Table 5 and the method of preparing a simvastatin immediate-release layer of example 25.

3) Compression and Coating

Press-coated tablets were prepared with a compressor for the production of osmotic core tablet (RUD-1: Kilian) by using the amlodipine core tablet and the composition containing atorvastatin as an inner core and an outer layer, respectively. The compression was performed at a speed of 30 revolutions per minute (rpm) to provide tablets with a hardness of 7-9 kp, a thickness of 6.0 mm and a diameter of 9.5 mm. A film coating layer was formed on the compressed tablets using Hi-coater (SFC-30N, Sejong mechanics, Korea).

### Example 33

### Preparation of a Amlodipine-Atorvastatin Blister Package Kits

Amlodipine-atorvastatin blister package kits were provided from predetermined amounts of ingredients as shown in Table 5 and the method of preparation the amlodipine tablet and the atorvastatin tablet were packaged in one blister kit for co-administration of Example 29, with the exception that instead of filling into capsules, the amlodipine and atorvastatin tablets were packed in a blister package, with each blister containing an amlodipine tablet and a atorvastatin tablet for co-administration.

### Example 34

### Preparation of Nifedipine-Simvastatin Press-Coated Tablets

1) Preparation of Nifedipine Timed-Release Layer (Inner Core)

Predetermined amounts of nifedipine and polyethyleneglycol 6000 as shown in Table 5 were mixing and making a solid dispersion. The solid dispersion was sieved with a No. 35 sieve, and mixed using a double cone mixer and mixed with microcrystalline cellulose, lauryl sodium sulfate with a double cone mixer. The granules were mixed with carbomer 71G powders, and mixed with magnesium stearate with a double cone mixer. The resulting mixture was compressed using a rotary compressor (MRC-33: Sejong) at a speed of 30 revolutions per minute (rpm) to provide tablets with a hardness of 7-9 kp, a thickness of 3.0 mm and a diameter of 5.5 mm. After the tablets were compressed, a film coating layer was formed on the compressed tablets by spraying a solution of hydroxypropylmethyl cellulose phthalate and acetylated monoglyceride in a 1:1 mixture of ethanol and methylene chloride using Hi-coater (SFC-30N, Sejong mechanics, Korea), thereby producing core tablets.

2) Preparation of a Simvastatin Immediate-Release Layer

A simvastatin immediate-release layer was prepared from predetermined amounts of ingredients as shown in Table 5 and the method of preparing a simvastatin immediate-release layer of example 1

3) Compression and Coating

Press-coated tablets were prepared with a compressor for the production of press-coated tablet (RUD-1: Kilian) by using the nifedipine core tablet and the composition containing simvastatin as an inner core and an outer layer, respectively. The compression was performed at a speed of 30 revolutions per minute (rpm) to provide tablets with a hardness of 7-9 kp, a thickness of 6.0 mm and a diameter of 9.5 mm. A film coating layer was formed on the compressed tablets using Hi-coater (SFC-30N, Sejong mechanics, Korea).

### Example 35

### Preparation of Nifedipine-Atorvastatin Press-Coated Tablets

Nifedipine-atorvastatin press-coated tablets were prepared from predetermined amounts of ingredients as shown in Table 5 and the method of preparation of Example 34, with the exception that atorvastatin was used as statin instead of simvastatin.

TABLE 5

| | | Amounts (mg/tablet) | | | | | |
|---|---|---|---|---|---|---|---|
| | | Examples | | | | | |
| Ingredients | | 30 | 31 | 32 | 33 | 34 | 35 |
| Timed- | Amlodipine besylate | 6.94 | 6.94 | 6.94 | 6.94 | — | — |
| release | Nifedipine | — | — | — | — | 30 | 30 |
| layer | Microcrystalline | 57.46 | 127.06 | 62.06 | 57.46 | 22.5 | 22.5 |
| | cellulose | | | | | | |
| | Dicalcium phosphate | — | 30 | — | — | — | — |
| | Sodium chloride | — | — | 10 | — | — | — |
| | Hydroxypropylmethyl | 3 | 6 | — | 3 | — | — |
| | cellulose | | | | | | |
| | Carbomer 71G¹⁾ | 10 | 15 | — | 10 | 10 | 10 |
| | Hydroxypropylmethyl | 10 | 12 | — | 10 | 10 | 10 |
| | cellulose phthalate | | | | | | |
| | Eudragit RS30D²⁾ | — | — | 12 | — | — | — |
| | Acetylated monoglyceride | 2 | 2 | — | 2 | 2 | 2 |
| | Triethyl citrate | — | — | 3 | — | — | — |
| | Magnesium stearate | 0.6 | 1 | 1 | 0.6 | 0.5 | 0.5 |
| Immediate- | Simvastatin | — | — | — | — | 20 | — |
| release | Atorvastatin | 10.85 | 10.85 | 10.85 | 10.85 | — | 10.85 |
| layer | Microcrystalline | 20 | — | 80 | 10 | 100 | 102.61 |
| | cellulose | | | | | | |
| | D-mannitol | — | — | — | 63.61 | — | — |
| | Lactose | 49.61 | — | 160.61 | — | 160.46 | 160 |
| | Corn Starch | 10 | — | 20 | — | 20 | 20 |
| | Sodium starch glycolate | 2 | — | 2 | 2 | 4 | 4 |
| | Butylated hydroxyanisole | 0.04 | 0.05 | 0.04 | 0.04 | 0.04 | 0.04 |
| | Hydroxypropy-cellulose | 3 | — | 6 | 3 | 6 | 6 |
| | Colloidal Silicon | 2 | — | 2 | 2 | 2 | 2 |
| | dioxide³⁾ | | | | | | |
| | Citric acid | 2 | — | 2 | 2 | 2 | 2 |
| | Magnesium stearate | 0.5 | — | 1.5 | 0.5 | 1.5 | 1.5 |
| | Hydroxypropylmethyl | — | 15 | — | 4 | — | — |
| | cellulose 2910 | | | | | | |
| | Colloidal silicon | — | 2.1 | — | — | — | — |
| | dioxide³⁾ | | | | | | |
| | Polyethylene glycol 6000 | — | 2 | — | 1 | — | — |
| | Titanium oxide | — | 1 | — | 0.8 | — | — |
| | Talc | — | 1 | — | 0.2 | — | — |
| Coating | Hydroxypropylmethyl | — | 3 | 6 | — | 6 | 6 |
| layer | cellulose 2910 | | | | | | |
| | Hydroxypropyl cellulose | — | 3 | 6 | — | 6 | 6 |
| | Titanium oxide | — | 1 | 2.5 | — | 2.5 | 2.5 |
| | Talc | — | 1 | 1.5 | — | 1.5 | 1.5 |
| | Ethanol | — | (48) | (80) | — | (80) | (80) |
| | Purified water | — | (12) | (20) | — | (20) | (20) |

| | | | | | | |
|---|---|---|---|---|---|---|
| Empty Capsule | 65 | — | — | — | — | — |
| Total | 255 | 240 | 396 | 190 | 443 | 436 |

¹⁾Carbomer 71G-Main ingredient: Carboxyvinylpolymer (Lubrizol)
²⁾Eugragit RS 30D-Main ingredient: Ammonio methacrylate copolymer (BASF)
³⁾Colloidal silicon dioxide-Commercial name: Aerosil 200 (Degussa)

### Example 36

### Preparation of Felodipine-Atorvastatin Two-Phase Matrix Capsules

1) Preparation of Felodipine Timed-Release Layer (Tablet)

Predetermined amounts of felodipine and microcrystalline cellulose as shown in Table 6 were sieved with a No. 35 sieve, and mixed using a double cone mixer. The mixture was placed into a fluidized-bed granulator (GPCG 1: Glatt), and sprayed with a binder solution (an aqueous solution of hydroxypropylmethyl cellulose) to prepare granules, and dried. After the granules were dried, they were coated by spraying a solution of hydroxypropylmethyl cellulose phthalate in a 1:1 mixture of ethanol and methylene chloride. The coated granules were mixed with magnesium stearate using a double cone mixer.

The granules were mixed with carbomer 71G powders, and mixed with magnesium stearate with a double cone mixer. The resulting mixture was compressed using a rotary compressor (MRC-33: Sejong) at a speed of 30 revolutions per minute (rpm) to provide tablets with a hardness of 7-9 kp, a thickness of 3.0 mm and a diameter of 5.5 mm. After the tablets were compressed, film coating layer was formed on the compressed tablets by spraying a solution of hydroxypropylmethyl cellulose phthalate in a 1:1 mixture of ethanol and methylene chloride using Hi-coater (SFC-30N, Sejong mechanics, Korea), thereby producing tablets.

2) Preparation of Atorvastatin Immediate-Release Layer

The atorvastatin immediate-release layer was prepared from predetermined amounts of ingredients as shown in Table 7 and the method of preparation of a simvastatin immediate-release layer of Example 22.

3) Capsule Filling

The felodipine tablets prepared in the aforementioned processes 1) and the atorvastatin tablets prepared in the aforementioned processes 2) were filled in No. 2 gelatin hard capsules using a capsule filling machine.

### Example 37

### Preparation of Barnidipine-Lovastatin Two-Phase Matrix Capsules

1) Preparation of Barnidipine Timed-Release Layer (Inner Core)

Predetermined amounts of barnidipine HCl and microcrystalline cellulose as shown in Table 6 were sieved with a No. 35 sieve, and mixed using a double cone mixer. The mixture was introduced into a high-speed mixer, combined with Kollicoat SR30D and granulated using an oscillator with a No. 20 sieve. After the granules were dried, they were ground with a No. 20 sieve. The sized granules were mixed with magnesium stearate using a double cone mixer and compressed using a rotary compressor (MRC-33; Sejong) at a rate of 30 revolutions per minute (rpm) to provide tablets with a hardness of 7-9 kp, a thickness of 3.0 mm and a diameter of 5.5 mm, which was used as core tablets.

2) Preparation of a Lovastatin Immediate-Release Layer

Predetermined amounts of lovastatin, microcrystalline cellulose and mannitol as shown in Table 7 were sieved with a No. 35 sieve and mixed using a high-speed mixer. A binder solution was prepared by dissolving hydroxypropyl cellulose and citric acid in water, and combined with the mixture containing the main ingredients. Thus obtained mixture was granulated using an oscillator with a No. 20 sieve, dried at 60° C. using a steam dryer, ground with a No. 20 sieve. The resulting mixture was mixed with butylatedhydroxyanisole, sodium starch glycolate and colloidal silicon dioxide, and finally mixed with magnesium stearate using a double cone mixer. The resulting mixture was compressed using a rotary compressor (MRC-33: Sejong) at a speed of 30 revolutions per minute (rpm) to provide tablets with a hardness of 7-9 kp, a thickness of 5.0 mm and a diameter of 5.5 mm, thereby producing tablets.

3) Capsule Filling

The barnidipine tablets prepared in the aforementioned processes 1) and the simvastatin tablets prepared in the aforementioned processes 2) were filled in No. 2 hydroxypropylmethyl cellulose hard capsules using a capsule filling machine.

### Example 38

### Preparation of Benidipine-Pitavastatin Two-Phase Matrix Capsules

1) Preparation of Benidipine Timed-Release Layer (Tablet)

A benidipine timed-release layer was prepared from predetermined amounts of ingredients as shown in Table 6 and the method of preparation for felodipine timed-release layer of example 36, with the exception that benidipine was used instead of felodipine.

2) Preparation of a Pitavastatin Immediate-Release Layer (Granule)

A pitavastatin immediate-release layer was prepared from predetermined amounts of ingredients as shown in Table 7 and the method of preparation of a simvastatin immediate-release layer of Example 1, with the exception that pitavastatin was used instead of simvastatin.

3) Capsule Filling

The benidipine tablets prepared in the aforementioned processes 1) and the pitavastatin granules prepared in the aforementioned processes 2) were filled into No. 1 gelatin hard capsules using a capsule filling machine.

### Example 39

### Preparation of Cilnidipine-Pravastatin Two-Phase Matrix Capsules

1) Preparation of Cilnidipine Timed-Release Layer (Tablet)

A cilnidipine timed-release layer was prepared from predetermined amounts of ingredients as shown in Table 6 and the method of preparing a barnidipine timed-release layer of Example 37, with the exception that cilnidipine was used instead of barnidipine.

2) Preparation of a Pravastatin Immediate-Release Layer (Granule)

A pravastatin immediate-release layer was prepared from predetermined amounts of ingredients as shown in Table 7 and the method of preparing a simvastatin immediate-release layer of Example 11, with the exception that pravastatin was used instead of simvastatin.

3) Capsule Filling

The cilnidipine tablets prepared in the aforementioned processes 1) and the pravastatin granule prepared in the aforementioned processes 2) were filled in No. 1 hydroxypropylmethyl cellulose hard capsules using a capsule filling machine.

### Example 40

### Preparation of Isradipine-Fluvastatin Two-Phase Matrix Capsules

1) Preparation of an Isradipine Timed-Release Layer (Pellet)

Predetermined amounts of isradipine and microcrystalline cellulose as shown in Table 6 were sieved with a No. 35 sieve, and mixed using a double cone mixer. A binder solution was prepared by dissolving hydroxypropylmethyl cellulose. The mixture and crystalline sucrose were placed into a CF granulator, and sprayed with the binder solution to prepare pellets. Then, crystalline sucrose was placed in a CF granulator and sprayed with the binder solution. The pellets were dried at 50° C. until the LOD (loss on drying) of the pellets dropped to 2% to prepare isradipine-containing core pellets. Pellet coating was formed on the predetermined pellets by spraying a solution of hydroxypropylmethyl cellulose in a 1:1 mixture of ethanol and methylene chloride using Hi-coater (SFC-30N, Sejong mechanics, Korea). The obtained pellets were coated by spraying with a solution of hydroxypropylmethyl cellulose phthalate dissolved in a 1:1 mixture of ethanol and methylene chloride using Hi-coater (SFC-30N, Sejong mechanics, Korea).

2) Preparation of a Fluvastatin Immediate-Release Layer (Granules)

A fluvastatin immediate-release layer was prepared from predetermined amounts of ingredients as shown in Table 7 and the method of preparing a simvastatin immediate-release layer of Example 1, with the exception that fluvastatin was used instead of simvastatin.

3) Capsule Filling

The final products of both processes 1 and 2 were filled into No. 1 gelatin hard capsules using a capsule filling machine.

### Example 41

### Preparation of Manidipine-Rosuvastatin Two-Phased Matrix Capsules

1) Preparation of Manidipine Timed-Release Layer (Granules)

Predetermined amounts of manidipine HCl and microcrystalline cellulose as shown in Table 6 were sieved with a No. 35 sieve, and mixed using a double cone mixer. The mixture was placed into a fluidized-bed granulator (GPCG 1: Glatt), and sprayed with a binder solution (an aqueous solution of hydroxypropylmethyl cellulose) to prepare granules, and dried. After the granules were dried, they were coated by spraying a solution of hydroxypropylmethyl cellulose phthalate in a 1:1 mixture of ethanol and methylene chloride. The coated granules were mixed with magnesium stearate using a double cone mixer.

2) Preparation of a Lovastatin Immediate-Release Layer (Tablets)

Predetermined amounts of lovastatin, microcrystalline cellulose and mannitol as shown in Table 7 were sieved with a No. 35 sieve and mixed using a high-speed mixer. A binder solution was prepared by dissolving hydroxypropyl cellulose and citric acid in water and combining with the mixture of the main ingredients. Thus obtained mixture was combined, granulated using an oscillator with a No. 20 sieve, dried at 60° C. using a steam dryer, ground with a No. 20 sieve, and mixed with butylatedhydroxyanisole, sodium starch glycolate, sillion dioxide and magnesium stearate.

The final composition was compressed using a rotary compressor (MRC-33: Sejong) at a speed of 30 revolutions per minute (rpm) to provide tablets with a hardness of 7-9 kp, a thickness of 3.0 mm and a diameter of 5.5 mm, thereby producing lovastatin immediate-release layer (tablets).

3) Mixing and Capsule Filling

The final products of both processes 1 and 2 were filled into No. 1 gelatin hard capsules using a capsule filling machine.

### Example 42

### Preparation of Nicardipine-Rosuvastatin Press-Coated Tablets

1) Preparation of Nicardipine Delayed Release Layer (Inner Core)

The nicardipine delayed release layer was prepared from predetermined amounts of ingredients as shown in Table 6 and a method of preparation a felodipine timed-release layer of example 36, part 1), with the exception that nicardipine was used instead of felodiopine.

2) Preparation of a Rosuvastatin Rapid Release Layer

The rosuvastatin rapid release layer was prepared from predetermined amounts of ingredients as shown in Table 7 and the method of preparation a rosuvastatin rapid release layer of Example 1, with the exception that rosuvastatin was used instead of simvastatin.

3) Compression and Coating

Press-coated tablets were prepared with a compressor for the production of press-coated tablet (RUD-1: Kilian) by using the nicardipine core tablet and the composition containing rosuvastatin as an inner core and an outer layer, respectively. The compression was performed at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea).

TABLE 6

| | | Amounts (mg/tablet) | | | | | | |
|---|---|---|---|---|---|---|---|---|
| | | Examples | | | | | | |
| Ingredients | | 36 | 37 | 38 | 39 | 40 | 41 | 42 |
| Delayed- | Felodipine | 5 | — | — | — | — | — | — |
| release | Barnidipine HCl | — | 10 | — | — | — | — | — |
| layer | Benidipine | — | — | 4 | — | — | — | — |
| | Cilnidipine | — | — | — | 10 | — | — | — |
| | Isradipine | — | — | — | — | 25 | — | — |
| | Manidipine HCl | — | — | — | — | — | 10 | — |
| | Nicardipine HCl | — | — | — | — | — | — | 20 |
| | Microcrystalline | 72.25 | 69.25 | 73.25 | 69.25 | 32 | 72.25 | 54.25 |
| | cellulose | | | | | | | |
| | Crystalline sucrose | — | — | — | — | 20 | — | — |
| | Kollicoat SR30D¹⁾ | — | 20 | — | 20 | — | — | — |
| | Eudragit RS PO²⁾ | — | — | — | — | — | — | — |
| | Carbomer 71G³⁾ | 10 | — | 10 | — | — | — | 13 |
| | Hydroxypropyl | — | — | — | — | 5 | — | — |
| | cellulose | | | | | | | |
| | Hydroxypropylmethyl | 2 | — | 2 | — | — | 2 | 2 |
| | cellulose | | | | | | | |
| | Hydroxypropylmethyl | 10 | — | 10 | — | 15 | 15 | 15 |
| | cellulose phthalate | | | | | | | |
| | Magnesium stearate | 0.75 | 0.75 | 0.75 | 0.75 | — | 0.75 | 0.75 |

¹⁾Kollicoat SR30D-Main ingredient: Polyvinylacetate 30% suspension (BASF)
²⁾Eudragit RS PO-Main ingredient:: Ammonio methacrylate copolymer (BASF)
³⁾Carbomer 71G-Main ingredient: Carboxyvinylpolymer (Lubrizol)

TABLE 7

| | | Amounts (mg/tablet) | | | | | | |
|---|---|---|---|---|---|---|---|---|
| | | Examples | | | | | | |
| Ingredients | | 36 | 37 | 38 | 39 | 40 | 41 | 42 |
| Rapid- | Atorvastatin calcium | 21.69 | — | — | — | — | — | — |
| release | Fluvastatin sodium | — | — | — | — | 21.06 | — | — |
| layer | Lovastatin | — | 20 | — | — | — | 20 | — |
| | Pitavastatin calcium | — | — | 2 | — | — | — | — |
| | Pravastatin sodium | — | — | — | 10 | — | — | — |
| | Rosuvastatin calcium | — | — | — | — | — | — | 10.4 |
| | Microcrystalline | 48.31 | 50 | 68 | 60 | 51.44 | 29.46 | 59.6 |
| | cellulose | | | | | | | |
| | D-mannitol | 112.46 | 112.46 | 112.46 | 112.46 | 112.46 | 20 | 112.46 |
| | Sodium starch | 1 | 1 | 1 | 1 | 1 | 1 | 1 |
| | glycolate | | | | | | | |
| | Butylated | 0.04 | 0.04 | 0.04 | 0.04 | 0.04 | 0.04 | 0.04 |
| | hydroxyanisole | | | | | | | |
| | Hydroxypropyl | 5 | 5 | 5 | 5 | 5 | 5 | 5 |
| | cellulose | | | | | | | |
| | Colloidal silicon | 1 | 1 | 1 | 1 | 1 | 1 | 1 |
| | dioxide¹⁾ | | | | | | | |
| | Citric acid | 2 | 2 | 2 | 2 | 2 | 2 | 2 |
| | Magnesium stearate | 1.5 | 1.5 | 1.5 | 1.5 | 1.5 | 1.5 | 1.5 |
| Coating | Hydroxypropylmethyl | — | — | — | — | — | — | 2.6 |
| layer | cellulose 2910 | | | | | | | |
| | Hydroxypropyl | — | — | — | — | — | — | 2.6 |
| | cellulose | | | | | | | |
| | Titanium oxide | — | — | — | — | — | — | 2.3 |
| | Talc | — | — | — | — | — | — | 1.5 |
| | Ethanol | — | — | — | — | — | — | (64.8) |
| | Purified Water | — | — | — | — | — | — | (16.2) |

| | | | | | | | |
|---|---|---|---|---|---|---|---|
| Empty Capsule | 65 | 65 | 80 | 80 | 80 | 65 | — |
| Total | 358 | 358 | 373 | 373 | 350 | 245 | 302 |

¹⁾Colloidal silicon dioxide-Commercial name: Aerosil 200 (Degussa)

### Example 43

### Preparation of Nifidipine-Fluvastatin Press-Coated Tablets

1) Preparation of Nifedipine Timed-Release Layer (Inner Core)

A nifedipine timed-release layer was prepared from predetermined amounts of ingredients as shown in Table 8 and the method of preparation a felodipine timed-release layer of example 36, part 1), with the exception that nifedipine was used instead of felodipine.

2) Preparation of a Fluvastatin Immediate-Release Layer

The fluvastatin immediate-release layer was prepared from predetermined amounts of ingredients as shown in Table 9 and the method of preparation of the fluvastatin immediate-release layer of Example 1, with the exception that fluvastatin sodium was used instead of felodipine.

3) Compression and Coating

Press-coated tablets were prepared with a compressor for the production of press-coated tablet (RUD-1: Kilian) by using the nifedipine core tablet and the composition containing simvastatin as an inner core and an outer layer, respectively. The compression was performed at a speed of 30 revolutions per minute (rpm) to provide tablets with a hardness of 7-9 kp, a thickness of 6.0 mm and a diameter of 9.5 mm. Film coating layer was formed on the compressed tablets using Hi-coater (SFC-30N, Sejong mechanics, Korea).

### Example 44

### Preparation of Amlodipine-Pitavastatin Press-Coated Tablets

Amlodipine-pitavastatin press-coated tablets were prepared from predetermined amounts of ingredients as shown in Tables 8 and 9, and the method of preparing an amlodipine timed-release layer of example 20, with the exception that pitavastatin was used instead of simvastatin.

### Example 45

### Preparation of Amlodipine-Rosuvastatin Press-Coated Tablets

Amlodipine-rosuvastatin press-coated tablets were prepared from predetermined amounts of ingredients as shown in Tables 8 and 9, and the method of preparing an amlodipine timed-release layer of example 20, with the exception that rosuvastatin was used as statin instead of simvastatin.

### Example 46

### Preparation of Nimodipine-Pravastatin Coated Tablets

1) Preparation of a Nimodipine Timed-Release Layer (Tablets)

Predetermined amounts of nimodipine and microcrystalline cellulose as shown in Table 10 were sieved with a No. 35 sieve, and mixed using a double cone mixer. The mixture was placed into a fluidized-bed granulator (GPCG 1: Glatt), and sprayed with a binder solution (an aqueous solution of hydroxypropylmethyl cellulose) to prepare granules, and dried. After the granules were dried, they were coated by spraying a solution of hydroxypropylmethyl cellulose phthalate in a 1:1 mixture of ethanol and methylene chloride. The coated granules were mixed with magnesium stearate using a double cone mixer.

The granules were mixed with carbomer 71G powders, and mixed with magnesium stearate with a double cone mixer. The resulting mixture was compressed using a rotary compressor (MRC-33: Sejong) at a speed of 30 revolutions per minute (rpm) to provide tablets with a hardness of 7-9 kp, a thickness of 3.0 mm and a diameter of 7.0 mm, thereby producing tablets.

2) Coating of a Pravastatin Immediate-Release Layer

Nimodipine tablets from 1) were coated with a coating solution where pravastatin sodium salt is dissolved according to the component and content as shown in Table 9 using Hi-coater (SFC-30N, Sejong mechanics, Korea), thereby producing film-coating tablets.)

3) Coating

The immediate-release tablets from 2) were then coated with a coating solution where pravastatin sodium salt is dissolved according to the component and content as shown in Table 7 using Hi-coater (SFC-30N, Sejong mechanics, Korea), thereby producing film-coating tablets.

### Example 47

### Preparation of Nivaldipine-Pitavastatin Multi-Layered Tablets

1) Preparation of Nivaldipine Delayed Release Layer (Granules)

Predetermined amounts of nivaldipine and microcrystalline cellulose as shown in Table 8 were sieved with a No. 35 sieve, mixed using a double cone mixer, sprayed with a binder solution (an aqueous solution of hydroxypropylmethyl cellulose) to prepare granules, and dried. After the granules were dried, they were coated by spraying a solution of hydroxypropylmethyl cellulose phthalate in a 1:1 mixture of ethanol and methylene chloride. The coated granules were mixed with magnesium stearate using a double cone mixer. The granules were mixed with Carbomer 71G powders, and mixed with magnesium stearate with a double cone mixer.

2) Preparation of a Pitavastatin Immediate-Release Layer

The pitavastatin immediate-release layer was prepared from predetermined amounts of ingredients as shown in Table 9 by the method of preparation a pitavastatin immediate-release layer of Example 1, part 2) with the exception that pitavastatin calcium was used instead of simvastatin.

3) Compression and Coating

Press-coated tablets were prepared with a compressor for the production of multi-layered tablet (MRC-37T: Sejong) by using the pitavastatin in the 1st powder inlet and the composition containing nivaldipine in the 2nd powder inlet in order to minimize the contamination between layers. The compression was performed at a speed of 30 revolutions per minute (rpm) to provide tablets with a hardness of 7-9 kp, a thickness of 6.0 mm and a diameter of 9.5 mm. The film coating was formed on the compressed tablets using Hi-coater (SFC-30N, Sejong mechanics, Korea).

### Example 48

### Preparation of Nisoldipine-Lovastatin Multi-Layered Tablets

1) Preparation of Nisoldipine Timed-Release Layer (Granule)

Predetermined amounts of nisoldipine and microcrystalline cellulose as shown in Table 8 were sieved with a No. 35 sieve, mixed using a double cone mixer, sprayed with a binder solution (an aqueous solution of hydroxypropylmethyl cellulose) to prepare granules, and dried. After the granules were dried, they were coated by spraying a solution of Eudragit RS PO in a 1:1 mixture of ethanol and methylene chloride. The coated granules were mixed with magnesium stearate using a double cone mixer.

2) Preparation of a Lovastatin Immediate-Release Layer

A lovastatin immediate-release layer was prepared from predetermined amounts of ingredients as shown in Table 9 and the method of preparing a simvastatin immediate-release layer of example 1, with the exception that lovastatin was used instead of simvastatin.

3) Compression and Coating

Press-coated tablets were prepared with a compressor for the production of multi-layered tablet (MRC-37T: Sejong) by using the lovastatin in the 1st chamber and the composition containing nisoldipine in the 2nd chamber. The compression was performed at a speed of 30 revolutions per minute (rpm) to provide tablets with a hardness of 7-9 kp, a thickness of 6.0 mm and a diameter of 9.5 mm. A film coating was formed on the compressed tablets using Hi-coater (SFC-30N, Sejong mechanics, Korea).

### Example 49

### Preparation of a Nitrendipine-Pravastatin Blister Package Kits

1) Preparation of Nitrendipine Timed-Release Layer

The nitrendipine timed-release layer was prepared from predetermined amounts of ingredients as shown in Table 8 and the method of preparation a felodipine timed-release layer of Example 36, with the exception that nitrendipine was used instead of felodipine.

2) Preparation of a Pravastatin Immediate-Release Layer

Predetermined amounts of pravastatin sodium, microcrystalline cellulose and mannitol as shown in Table 9 were sieved with a No. 35 sieve and mixed using a high-speed mixer. A binder solution was prepared by dissolving hydroxypropyl cellulose and citric acid in water, and combined with the mixture containing the main ingredients. Thus obtained mixture was granulated using an oscillator with a No. 20 sieve, dried at 60° C. using a steam dryer, ground with a No. 20 sieve. The resulting mixture was mixed with butylatedhydroxyanisole, sodium starch glycolate and colloidal silicon dioxide, and finally mixed with magnesium stearate using a double cone mixer. The resulting mixture was compressed using a rotary compressor (MRC-33: Sejong) at a speed of 30 revolutions per minute (rpm) to provide tablets with a hardness of 7-9 kp, a thickness of 4.0 mm and a diameter of 8.5 mm, thereby producing tablets.

3) Packaging

The nitrendipine tablets prepared in the aforementioned process 1) and the pravastatin tablets prepared in the aforementioned process 2) were packaged in a blister pack with one nitrendipine and one pravastatin tablet per blister for co-administration and labeled for such use.

TABLE 8

| | | Amounts (mg/tablet) | | | | | | |
|---|---|---|---|---|---|---|---|---|
| | | Examples | | | | | | |
| Ingredients | | 43 | 44 | 45 | 46 | 47 | 48 | 49 |
| Delayed- | Nifedipine | 10 | — | — | — | — | — | — |
| release | Amlodipine besylate | | 6.94 | 6.94 | | | | |
| layer | Nimodipine | — | — | — | 30 | — | — | — |
| | Nivaldipine | — | — | — | — | 4 | — | — |
| | Nisoldipine | — | — | — | — | — | 10 | — |
| | Nitrendipine | — | — | — | — | — | — | 20 |
| | Microcrystalline | 67.25 | 40.46 | 40.46 | 142.25 | 73.25 | 72.25 | 42.25 |
| | cellulose | | | | | | | |
| | Dicalcium phosphate | | 16 | 16 | | | | |
| | Eudragit RS PO¹⁾ | — | — | — | — | — | 20 | — |
| | Acetylated | | 2 | 2 | | | | |
| | monoglyceride | | | | | | | |
| | Carbomer 71G²⁾ | 10 | 10 | 10 | 15 | 10 | — | 15 |
| | Hydroxypropylmethyl | 2 | 4 | 4 | 2 | 2 | 2 | 2 |
| | cellulose | | | | | | | |
| | Hydroxypropylmethyl | 10 | 10 | 10 | 10 | 10 | — | 10 |
| | cellulose phthalate | | | | | | | |
| | Magnesium stearate | 0.75 | 0.6 | 0.6 | 0.75 | 0.75 | 0.75 | 0.75 |

¹⁾Eudragit RS PO-Main ingredient:: Ammonio methacrylate copolymer (BASF)
²⁾Carbomer 71G-Main ingredient: Carboxyvinylpolymer (Lubrizol)

TABLE 9

| | | Amounts (mg/tablet) | | | | | | |
|---|---|---|---|---|---|---|---|---|
| | | Examples | | | | | | |
| Ingredients | | 43 | 44 | 45 | 46 | 47 | 48 | 49 |
| Rapid- | Atorvastatin calcium | — | — | — | — | — | — | — |
| release | Fluvastatin sodium | 21.06 | — | — | — | — | — | — |
| layer | Lovastatin | — | — | — | — | — | 20 | — |
| | Pitavastatin calcium | — | 2 | — | — | 2 | — | — |
| | Pravastatin sodium | — | — | — | 10 | — | — | 10 |
| | Rosuvastatin calcium | — | — | 20 | — | — | — | — |
| | Microcrystalline | 48.94 | 98 | 80 | — | 60 | 50 | 60 |
| | cellulose | | | | | | | |
| | D-Mannitol | 112.46 | — | — | — | 112.46 | 112.46 | 112.46 |
| | Lactose | | 160.46 | 160.46 | | | | |
| | Corn starch | | 20 | 20 | | | | |
| | Sodium starch glycolate | 1 | 2 | 2 | — | 1 | 1 | 1 |
| | Butylated hydroxyanisole | 0.04 | 0.04 | 0.04 | — | 0.04 | 0.04 | 0.04 |
| | Hydroxypropyl cellulose | 5 | 6 | 6 | 13 | 5 | 5 | 5 |
| | Hydroxypropylmethyl | — | — | — | 13 | — | — | — |
| | cellulose 2910 | | | | | | | |
| | Colliodal silicon | 1 | 2 | 2 | — | 1 | 1 | 1 |
| | dioxide¹⁾ | | | | | | | |
| | Citric acid | 2 | 2 | 2 | — | 2 | 2 | 2 |
| | Magnesium stearate | 1.5 | 1.5 | 1.5 | — | 1.5 | 1.5 | 1.5 |
| | Titanium oxide | — | — | — | 11.5 | — | — | — |
| | Talc | — | — | — | 7.5 | — | — | — |
| | Ethanol | — | — | — | (208) | — | — | — |
| | Purified Water | — | — | — | (52) | — | — | — |
| Coating | Hydroxypropylmethyl | 2.6 | 6 | 6 | 3 | 2.6 | 2.6 | — |
| layer | cellulose 2910 | | | | | | | |
| | Hydroxypropyl cellulose | 2.6 | 6 | 6 | 3 | 2.6 | 2.6 | — |
| | Titanium oxide | 2.3 | 2.5 | 2.5 | 2 | 2.3 | 2.3 | — |
| | Talc | 1.5 | 1.5 | 1.5 | 1 | 1.5 | 1.5 | — |
| | Ethanol | (64.8) | (80) | (80) | (60.0) | (64.8) | (64.8) | — |
| | Purified water | (16.2) | (20) | (20) | (15.0) | (16.2) | (16.2) | — |

| | | | | | | | |
|---|---|---|---|---|---|---|---|
| Total | 302 | 400 | 400 | 264 | 294 | 307 | 283 |

¹⁾Colloidal silicon dioxide-Commercial name: Aerosil 200 (Degussa)

### Example 50

### Preparation of (S)-Amlodipine-Simvastatin Press-Coated Tablets

1) Preparation of a (S)-Amlodipine Timed-Release Layer (Inner Core)

Predetermined amounts of (S)-amlodipine besylate and microcrystalline cellulose as shown in Table 10 were sieved with a No. 35 sieve, and mixed using a double cone mixer. The mixture was placed into a fluidized-bed granulator (GPCG 1: Glatt), and sprayed with a binder solution (an aqueous solution of hydroxypropylmethyl cellulose) to prepare granules, and dried. The granules were mixed with carbomer 71G powders, and mixed with magnesium stearate with a double cone mixer. The resulting mixture was compressed using a rotary compressor (MRC-33: Sejong) at a speed of 30 revolutions per minute (rpm) to provide tablets with a hardness of 7-9 kp, a thickness of 4.0 mm and a diameter of 8.0 mm, thereby producing tablets.

2) Preparation of a Simvastatin Immediate-Release Layer

Predetermined amounts of simvastatin, microcrystalline cellulose and mannitol as shown in Table 10 were sieved with a No. 35 sieve and mixed using a high-speed mixer for 10 min. A binder solution prepared by dissolving hydroxypropyl cellulose and citric acid in water, and combined with the mixture containing the main ingredients. The resulting mixture was mixed with butylatedhydroxyanisole, sodium starch glycolate and colloidal silicon dioxide for 10 min and finally mixed with magnesium stearate for 4 min using a double cone mixer.

3) Compression and Coating

Press-coated tablets were prepared with a compressor for the production of core tablet (RUD-1: Kilian) by using the amlodipine core tablet and the composition containing atorvastatin as an inner core and an outer layer, respectively. The compression was performed at a speed of 30 revolutions per minute (rpm) to provide tablets with a hardness of 7-13 kp, a thickness of 6.0 mm and a diameter of 9.5 mm. A film coating layer as shown table 8. was formed on the compressed tablets using Hi-coater (SFC-30N, Sejong mechanics, Korea).

### Example 51

### Preparation of (S)-Amlodipine-Simvastatin Press-Coated Tablets

(S)-amlodipine-simvastatin press-coated tablets were prepared from predetermined amounts of ingredients as shown in Table 10 and the method of preparation a (S)-amlodipine-simvastatin press-coated tablets of Example 50, with the exception that polycopolymer(methacrylate, methylmethacrylate) enteric coating was included.

### Example 52

### Preparation of (S)-Amlodipine-Simvastatin Capsules

1) Preparation of a (S)-Amlodipine Timed-Release Layer (Tablets)

Predetermined amounts of (S)-amlodipine besylate and microcrystalline cellulose as shown in Table 10 were sieved with a No. 35 sieve, and mixed using a double cone mixer. The mixture was placed into a fluidized-bed granulator (GPCG 1: Glatt), and sprayed with a binder solution (an aqueous solution of hydroxypropylmethyl cellulose) to prepare granules, and dried. The granules were mixed with carbomer 71G powders, and mixed with magnesium stearate with a double cone mixer. The resulting mixture was compressed using a rotary compressor (MRC-33: Sejong) at a speed of 30 revolutions per minute (rpm) to provide tablets with a hardness of 6-10 kp, a thickness of 3.0 mm and a diameter of 5.5 mm. After the tablets were compressed, a film coating layer was formed on the compressed tablets by copolymer(methacrylate, polymethacrylate) coating using Hi-coater (SFC-30N, Sejong mechanics, Korea), thereby producing tablets.

2) Preparation of a Simvastatin Immediate-Release Layer (Tablets)

Predetermined amounts of simvastatin sodium, microcrystalline cellulose and mannitol as shown in Table 10 were sieved with a No. 35 sieve and mixed using a high-speed mixer for 10 min. A binder solution prepared by dissolving hydroxypropyl cellulose and citric acid in water, and combined with the mixture containing the main ingredients. The resulting mixture was mixed with butylatedhydroxyanisole, sodium starch glycolate and colloidal silicon dioxide for 10 min, and finally mixed with magnesium stearate for 4 min using a double cone mixer. The resulting mixture was compressed using a rotary compressor (MRC-33: Sejong) at a speed of 30 revolutions per minute (rpm) to provide tablets with a hardness of 6-10 kp, a thickness of 3.0 mm and a diameter of 5.5 mm. They were coated by a film layer as shown in table 8, thereby producing tablets.

3) Capsule Filling

The (S)-amlodipine tablets prepared in the aforementioned process 1) and the simvastatin tablets prepared in the aforementioned process 2) filled into No. 3 gelatin hard capsules using a capsule filling machine simultaneously.

### Example 53

### Preparation of Capsules Comprising with (S)-Amlodipine Tablets-Simvastatin Tablets

1) Preparation of a (S)-Amlodipine Timed-Release Layer (Tablets)

Predetermined amounts of (S)-amlodipine besylate and microcrystalline cellulose as shown in Table 10 were sieved with a No. 35 sieve, and mixed using a double cone mixer. The mixture was placed into a fluidized-bed granulator (GPCG 1: Glatt), and sprayed with a binder solution (an aqueous solution of hydroxypropylmethyl cellulose) to prepare granules, and dried. The granules were mixed with carbomer 71G powders, and mixed with magnesium stearate with a double cone mixer. The resulting mixture was compressed using a rotary compressor (MRC-33: Sejong) at a speed of 30 revolutions per minute (rpm) to provide tablets with a hardness of 7-9 kp, a thickness of 4.0 mm and a diameter of 8.0 mm. After the tablets were compressed, a film coating layer was formed on the compressed tablets by copolymer(methacrylate, polymethacrylate) coating using Hi-coater (SFC-30N, Sejong mechanics, Korea), thereby producing tablets.

2) Preparation of a Simvastatin Immediate-Release Layer (Granules)

Predetermined amounts of simvastatin, microcrystalline cellulose and mannitol as shown in Table 10 were sieved with a No. 35 sieve and mixed using a high-speed mixer for 10 min. A binder solution prepared by dissolving hydroxypropyl cellulose and citric acid in water, and combined with the mixture containing the main ingredients. The resulting mixture was mixed with butylatedhydroxyanisole, sodium starch glycolate and colloidal silicon dioxide for 10 min and finally mixed with magnesium stearate for 4 min using a double cone mixer.

3) Capsule Filling

The (S)-amlodipine tablets prepared in the aforementioned process 1) and the simvastatin granules prepared in the aforementioned process 2) were filled in No. 1 hydroxymethyl cellulose hard capsules using a capsule filling machine simultaneously.

### Example 54

### Preparation of a (S)-Amlodipine-Simvastatin Blister Package Kits

Preparation of (S)-Amlodipine-Simvastatin Blister Package Kits were Provided from predetermined amounts of ingredients as shown in Table 10 and the method of preparation of the (S)-amlodipine tablet and the simvastatin tablet were packaged in one blister kit for co-administration of Example 52, with the exception that the (S)-amlodipine tablet and the simvastatin tablet were packaged in a blister pack for co-administration (as in Example 49) instead of capsule filling.

### Example 55

### Preparation of (S)-Amlodipine-Simvastatin Coating Tablets

1) Preparation of a (S)-amlodipine timed-release layer (tablet)

Predetermined amounts of (S)-amlodipine besylate and microcrystalline cellulose as shown in Table 10 were sieved with a No. 35 sieve, and mixed using a double cone mixer. The mixture was placed into a fluidized-bed granulator (GPCG 1: Glatt), and sprayed with a binder solution (an aqueous solution of hydroxypropylmethyl cellulose) to prepare granules, and dried. The granules were mixed with carbomer 71G powders, and mixed with magnesium stearate with a double cone mixer. The resulting mixture was compressed using a rotary compressor (MRC-33: Sejong) at a speed of 30 revolutions per minute (rpm) to provide tablets with a hardness of 7-9 kp, a thickness of 4.0 mm and a diameter of 8.0 mm. After the tablets were compressed, a film coating layer was formed on the compressed tablets by copolymer(methacrylate, polymethacrylate) coating using Hi-coater (SFC-30N, Sejong mechanics, Korea), thereby producing tablets.

2) Preparation of a Simvastatin Coating Solution

Predetermined amounts of simvastatin, butylatedhydroxyanisole, hydroxypropylmethylcellulose, colloidal silicon dioxide as shown in Table 10 were dissolved and dispersed in a mixture of ethanol and methylene chloride to make a simvastatin coating solution.

3) Primary Coating

After the aforemention (S)-amlodipine tablets were compressed, primary coating layer was formed on the compressed tablets by spraying a simvastatin coating solution using Hi-coater (SFC-30N, Sejong mechanics, Korea), thereby producing primary coated tablets.

4) Secondary Coating

After preparation of a secondary coating solution were provided by predetermined amounts of ingredients as shown in Table 10 of a coating layer, film coating layer was formed on the primary coated tablets by spraying a secondary coating solution, thereby producing film coated tablets.

TABLE 10

| | | Amounts (mg/tablet) | | | | | |
|---|---|---|---|---|---|---|---|
| | | Examples | | | | | |
| Ingredients | | 50 | 51 | 52 | 53 | 54 | 55 |
| Delayed- | (S)-Amlodipine besylate | 3.74 | 3.74 | 3.74 | 3.74 | 3.74 | 3.74 |
| release | Microcrystalline cellulose | 48.26 | 48.26 | 48.26 | 48.66 | 48.26 | 152.68 |
| layer | Kollicoat SR30D¹⁾ | — | — | — | — | — | — |
| | EudragitRS PO²⁾ | — | — | — | — | — | — |
| | Carbomer 71G³⁾ | 6.0 | 6.0 | 6.0 | 6.0 | 6.0 | 18.0 |
| | Hydroxypropyl cellulose | 1.5 | 1.5 | 1.5 | 1.5 | 1.5 | 4.5 |
| | Poly(methacrylate, | — | 6.6 | 6.6 | 6.6 | 6.6 | 20.0 |
| | methylmethacrylate) | | | | | | |
| | copolymer | | | | | | |
| | Magnesium stearate | 0.5 | 0.5 | 0.5 | 0.5 | 0.5 | 1.5 |
| | Purified water | (22) | (88) | (88) | (88) | (88) | (270) |
| Immediate- | Simvastatin | 20 | 20 | 20 | 20 | 20 | 20.0 |
| release | Microcrystalline cellulose | 123.0 | 123.0 | 31.0 | 31.0 | 31.0 | — |
| layer | D-mannitol | 247.42 | 247.42 | 61.5 | 61.5 | 61.5 | — |
| | Sodium starch glycolate | 13.0 | 13.0 | 3.2 | 3.2 | 3.2 | — |
| | Butylated hydroxyanisole | 0.08 | 0.08 | 0.04 | 0.04 | 0.04 | 0.08 |
| | Hydroxypropyl cellulose | 13.0 | 13.0 | 3.2 | 3.2 | 3.2 | — |
| | Hydroxypropylmethyl | — | — | — | — | 13 | 10 |
| | cellulose 2910 | | | | | | |
| | Colloidal silicon dioxide⁴⁾ | — | — | 0.5 | 0.5 | 0.5 | 5.0 |
| | Citric acid | 3.5 | 3.5 | 1.0 | 1.0 | 1.0 | — |
| | Magnesium stearate | 4.0 | 4.0 | 0.56 | 0.56 | 0.56 | — |
| | Purified water | (80.0) | (80.0) | (20.0) | (20.0) | (20.0) | — |
| | Ethanol | — | — | — | — | — | (200.0) |
| | Methylene chloride | — | — | — | — | — | (500.0) |
| Coating | Hydroxypropylmethyl | — | — | — | — | — | — |
| layer | cellulose 2910 | | | | | | |
| | Opadry⁵⁾ | 17 | 174 | 4.4 | — | 4.4 | 4.5 |
| | Titanium oxide | — | — | — | — | — | — |
| | Talc | — | — | — | — | — | — |
| | Ethanol | (115.84) | (115.84) | (36.0) | — | (36.0) | (36.0) |
| | Purified water | (28.96) | (28.96) | (9.0) | — | (9.0) | (9.0) |

| | | | | | | |
|---|---|---|---|---|---|---|
| Empty Capsule | — | — | 50 | 65 | — | — |
| Total | 501 | 508 | 242 | 253 | 192 | 240 |

¹⁾Kollicoat SR30D-Main ingredient: Polyvinylacetate 30% suspension (BASF)
²⁾Eugragit RS 30D-Main ingredient: Ammonio methacrylate copolymer (BASF)
³⁾Carbomer 71G-Main ingredient: Carboxyvinylpolymer (Lubrizol)
⁴⁾Colloidal silicon dioxide-Commercial name: Aerosil 200 (Degussa)
⁵⁾Opadry-Main ingredients: Hydroxypropylmethyl cellulose, titanium oxide, Polyethylene glycol,Colorant (Colorcon)

### Example 56

### Preparation of Capsules Comprising with (S)-Amlodipine Tablets-Atorvastatin Tablets

1) Preparation of (S)-Amlodipine Delayed Release Layer (Tablets)

The (S)-amlodipine delayed release layer was prepared from predetermined amounts of ingredients as shown in Table 11 and a method of preparation of a (S)-amlodipine timed release layer of Example 53, part 1).

2) Preparation of Atorvastatin Immediate-Release Layer (Pellets)

Predetermined amounts of atorvastatin calcium, butyratedhydroxyanizole and microcrystalline cellulose as shown in Table 11 were sieved with a No. 35 sieve and mixed using a high-speed mixer. With spraying the binder solution (aqueous solution of hydroxypropyl cellulose and citric acid) to the crystalline sucrose in the CF granulater, the mixture containing the main ingredients was mixed to produce pellets. The pellets were dried at 50° C. until reaching to NMT 2% LOD (loss on drying) to produce pellets of atorvastatin calcium.

3) Capsule Filling

The (S)-amlodipine tablets prepared in the aforementioned process 1) and the atorvastatin pellets prepared in the aforementioned process 2) were filled into No. 2 hydroxypropylmethylcellulose hard capsules simultaneously using a capsule filling machine.

### Example 57

### Preparation of Capsules Comprising with (S)-Amlodipine Pellets-Atorvastatin Tablet

1) Preparation of (S)-Amlodipine Delayed Release Layer (Pellet)

Predetermined amounts of (S)-amlodipine besylate and microcrystalline cellulose as shown in Table 11 were screened with a No. 35 sieve and mixed using a double cone mixer. With spraying the binder solution (aqueous solution of hydroxypropyl cellulose and citric acid) to the crystalline sucrose in the CF granulator, the mixture containing the main ingredients was mixed with the sucrose seed to produce pellets. The pellets were dried at 50° C. until reaching to NMT 2% LOD (loss on drying) to produce pellets of (S)-amlodipine besylate.

2) Preparation of Atorvastatin Rapid Release Layer (Tablet)

Predetermined amounts of atorvastatin calcium, microcrystalline cellulose and mannitol as shown in Table 11 were screened with a No. 20 sieve and mixed using a high speed mixer for 10 minutes. After spraying the binder solution (aqueous solution of hydroxypropyl cellulose and citric acid) to the mixture, the granules was prepared by granulation, drying and oscillation.

The atorvastatin granules were mixed with pre-screened ingredients of butylate hydroxyanisole, Sodium starch glycolate, colloidal silicon dioxide for 10 minutes and mixed again with screened with No. 35 sieve magnesium stearate for 4 minutes with double cone mixer.

The mixture was compressed using a rotary compressor (MRC-33: Sejong) at a speed of 30 resolutions per minutes (rpm) to provide tablets with a hardness of 6-10 kp, a thickness of 3.0 mm and a diameter of 5.5 mm. The tablet was coated with film coating layer with the composition as shown in table 11.

3) Capsule Filling

The (S)-amlodipine besylate pellets and atorvastatin tablets prepared in the aforementioned processes 1) and 2) are filled simultaneously into No. 2 hydroxypropylmethylcellulose hard capsules using a capsule filling machine.

### Example 58

### Preparation of Capsules Comprising (S)-Amlodipine Pellets-Atorvastatin Pellets

1) Preparation of (S)-Amlodipine Delayed Release Layer (Pellets)

The (S)-amlodipine delayed release layer was prepared from predetermined amounts of ingredients as shown in Table 11 and the method of preparation of a (s)-amlodipine immediate release layer of Example 57, part 1).

2) Preparation of Atorvastatin Immediate-Release Layer (Pellets)

The atorvastatin immediate-release layer was prepared from predetermined amounts of ingredients as shown in Table 11 and the method of preparation a atorvastain immediate-release layer (pellets) of Example 56, part 2).

3) Capsule Filling

The (S)-amlodipine pellets and atorvastatin pellets prepared in the aforementioned processes 1) and 2) are filled simultaneously into No. 2 hydroxypropylmethyl cellulose hard capsule using a capsule filling machine.

### Example 59

### Preparation of Capsules Comprising (S)-Amlodipine Granules-Atorvastatin Pellets

1) Preparation of (S)-Amlodipine Delayed Release Layer (Granules)

The (S)-amlodipine delayed release layer was prepared from predetermined amounts of ingredients as shown in Table 11. The screened ingredients of (S)-amlodipine besylate and microcrystalline cellulose with No. 35 sieve was mixed using double corn mixer, thereafter placed to fluidized bed granulator (GPCG 1: Glatt). The granules was coated with coating solution, Kollocoat SR 30D and dried.

2) Preparation of Atorvastatin Immediate-Release Layer (Pellets)

The atorvastatin immediate-release layer was prepared from predetermined amounts of ingredients as shown in Table 11 and the method of preparation a atorvastain immediate-release layer (pellets) of Example 56, part 2).

3) Capsule Filling

The (S)-amlodipine granules and atorvastatin pellets prepared in the aforementioned processes 1) and 2) are filled simultaneously into No. 2 hydroxypropylmethylcellulose hard capsule using a capsule filling machine.

### Example 60

### Preparation of Capsules Comprising (S)-Amlodipine Pellets-Atorvastatin Granules

1) Preparation of (S)-Amlodipine Delayed Release Layer (Pellets)

The (S)-amlodipine delayed release layer was prepared from predetermined amounts of ingredients as shown in Table 11 and the method of preparation of the (S)-amlodipine delayed release layer of Example 57, part 1).

2) Preparation of Atorvastatin Immediate-Release Layer (Pellets)

The atorvastatin immediate-release layer was prepared from predetermined amounts of ingredients as shown in Table 11 and the method of preparation a atorvastain immediate-release layer (pellets) of Example 56, part 2).

3) Capsule Filling

The (S)-amlodipine pellets and atorvastatin granules prepared in the aforementioned processes 1) and 2) are filled simultaneously into No. 2 hydroxypropylmethylcellulose hard capsule using a capsule filling machine.

TABLE 11

| | | Amounts (mg/tablet) | | | | |
|---|---|---|---|---|---|---|
| | | Examples | | | | |
| Ingredients | | 56 | 57 | 58 | 59 | 60 |
| delayed- | (S)-Amlodipine besylate | 3.74 | 3.74 | 3.74 | 3.74 | 3.74 |
| release layer | Crystalline sucrose | — | 40 | 40 | — | 40 |
| | Microcrystalline cellulose | 48.66 | 31.26 | 31.26 | 61.26 | 31.26 |
| | Kollicoat SR30D¹⁾ | — | — | — | — | — |
| | Carbomer 71G²⁾ | 6.0 | — | — | — | — |
| | Hydroxypropyl cellulose | 1.5 | 1.5 | 5 | — | 5 |
| | Poly (methacrylate, | 6.6 | — | — | — | — |
| | methylmethacrylate) copolymer | | | | | |
| | Magnesium stearate | 0.5 | — | — | — | — |
| | Purified water | (40) | (50) | (50) | (20) | (40) |
| rapid-release | Atorvastatin Calsium | (40) | (50) | (50) | (20) | (40) |
| layer | Microcrystalline cellulose | 40.27 | 55.31 | 40.27 | 40.27 | 55.31 |
| | D-mannitol | — | 62.46 | — | — | 112.46 |
| | Sodium starch glycolate | — | 1 | — | — | 1 |
| | Butylated hydroxyanisole | 0.04 | 0.04 | 0.04 | 0.04 | 0.04 |
| | Hydroxypropyl cellulose | 6 | 5 | 6 | 6 | 5 |
| | Colloidal silicon dioxide³⁾ | — | 1 | — | — | 1 |
| | Citric acid | — | 2 | — | — | 2 |
| | Magnesium stearate | | 1.5 | — | — | 1.5 |
| | Purified water | (60.0) | (50.0) | (60.0) | (60.0) | (60.0) |
| Coating layer | Hydroxypropylmethyl cellulose | — | 3.6 | — | — | — |
| | 2910 | | | | | |
| | Hydroxypropylcellulose | | 2.6 | | — | |
| | Titanium oxide | — | 2.3 | — | — | — |
| | Talc | — | 1.5 | — | — | — |
| | Ethanol | — | (64.8) | — | — | — |
| | Purified water | | (16.2) | — | — | — |

| | | | | | |
|---|---|---|---|---|---|
| Emply Capsule | 50 | 65 | 50 | 65 | 65 |
| Total | 225 | 305 | 238 | 258 | 345 |

¹⁾Kollicoat SR30D-Main ingredient: Polyvinylacetate 30% suspension (BASF)
²⁾Carbomer 71G-Main ingredient: Carboxyvinylpolymer (Lubrizol)
³⁾Colloidal silicon dioxide-Commercial name: Aerosil 200 (Degussa)

### Experimental Example 1

### Comparative Dissolution Profile Test

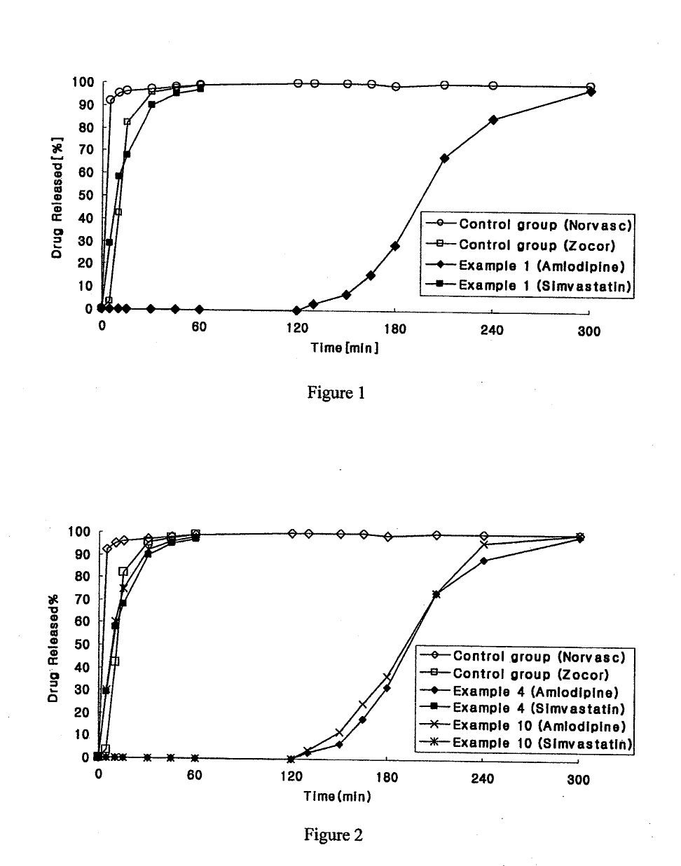

Comparative dissolution profile test was performed using an amlodipine/simvastatin tablets prepared in Example 1 and control drugs (Zocor®: simvastatin single pill, MSD, Norvasc: amlodipine single pill, Pfizer). In the case of dissolution profile test of amlodipine ingredient, the dissolution solution was changed from an artificial gastric juice to an artificial intestinal juice after 2 hours. The detailed process of dissolution profile test of each ingredient is described below, and the results are presented in FIG. 1.

When the dissolution profile test was performed under the conditions described below, with tablets according to the present invention, the simvastatin ingredient showed a nearly equivalent dissolution behavior as compared to that of the control drug (Zocor®), while the amlodipine ingredient showed a very delayed dissolution rate as compared to that of the control drug (Norvasc®). In the case of the amlodipine/simvastatin tablets according to the present invention, dissolution rates of amlodipine ingredient were all within 50% until one hour after the test began, which were far lower than that of the control drug (about 99%).

As described above, in the amlodipine/simvastatin tablet according to the present invention, amlodipine shows a far lower initial dissolution rate than simvastatin unlike the control drugs (i.e., amlodipine single pill), and thus the amlodipine/simvastatin tablet according to the present invention is less likely to be subject to the metabolism in liver ahead of simvastatin.

Test Method for Amlodipine:

Based on the general dissolution test method described in Korea Pharmacopoeia (8th revision); Test method: Paddle method, 75 rpm; Dissolution medium: 0.01 M Hydrochloric acid, 750 mL to two hours, pH 6.8 simulated intestinal fluid 1,000 mL (after 2 hr); Analysis method: UV-visible spectrophotometry (detected wavelength=maximum 240 μm).

Test Method for Simvastatin:

Based on the ‘Simvastatin tablet’ part in USP XXIX; Test method: Paddle method, 50 rpm; Dissolution solution: pH=7.0 buffer solution (0.01 M monobasic sodium phosphate solution containing sodium lauryl sulfate 0.5% wt/wt as surfactant), 900 mL; Analysis method: UV-visible spectrophotometry (detected wavelength=maximum 247 nm and minimum 257 nm).

### Experimental Example 2

### Comparative Dissolution Profile Test

Comparative dissolution profile test was performed using an amlodipine/simvastatin combined pharmaceutical formulation prepared in Examples 4 and 10 and control drugs (Zocor®: simvastatin single pill, Norvasc®: amlodipine single pill). The dissolution behavior of simvastatin and amlodipine was observed as described below, and in the case of dissolution profile test of amlodipine ingredient, the dissolution solution was changed from an artificial gastric juice to an artificial intestinal juice after 2 hours. Specific process of dissolution profile test of each ingredient is described below, and the results are presented in FIG. 2.

When the dissolution profile test was performed under the conditions described below in Examples 4 and 10, the simvastatin ingredient showed a nearly equivalent dissolution behavior as compared to that of the control drug (Zocor®), while the amlodipine ingredient showed a very delayed dissolution rate as compared to that of the control drug (Norvasc®). In the case of the amlodipine/simvastatin multi-layered tablet according to the present invention, dissolution rates of amlodipine ingredient were all within 50% until one hour after the test began, which were far lower than that of the control drug (about 99%).

As described above, in the amlodipine/simvastatin multi-layered tablet according to the present invention, amlodipine shows a far lower initial dissolution rate than simvastatin unlike the control drugs (i.e., amlodipine single pill), and thus the amlodipine/simvastatin multi-layered tablet according to the present invention is less likely to be subject to the metabolism in the liver ahead of simvastatin.

Test Method for Amlodipine:

Based on the general dissolution test method described in Korea Pharmacopoeia (8th revision); Test method: Paddle method), 75 rpm; Dissolution medium: 0.01 M Hydrochloric acid, 750 mL (to 2 hr), pH 6.8 simulated intestinal fluid 1,000 mL (after 2 hr); Analysis method: UV-visible spectrophotometry (detected wavelength=maximum 240 nm).

Test Method for Simvastatin:

Based on the ‘Simvastatin tablet’ part in USP XXIX; Test method: Paddle method, 50 rpm; Dissolution solution: pH=7.0 buffer solution (0.01 M monobasic sodium phosphate solution containing sodium lauryl sulfate 0.5% wt/wt as surfactant), 900 mL; Analysis method: UV-visible spectrophotometry (detected wavelength maximum 247 nm and minimum 257 nm).

### Experimental Example 3

### Comparative Dissolution Profile Test

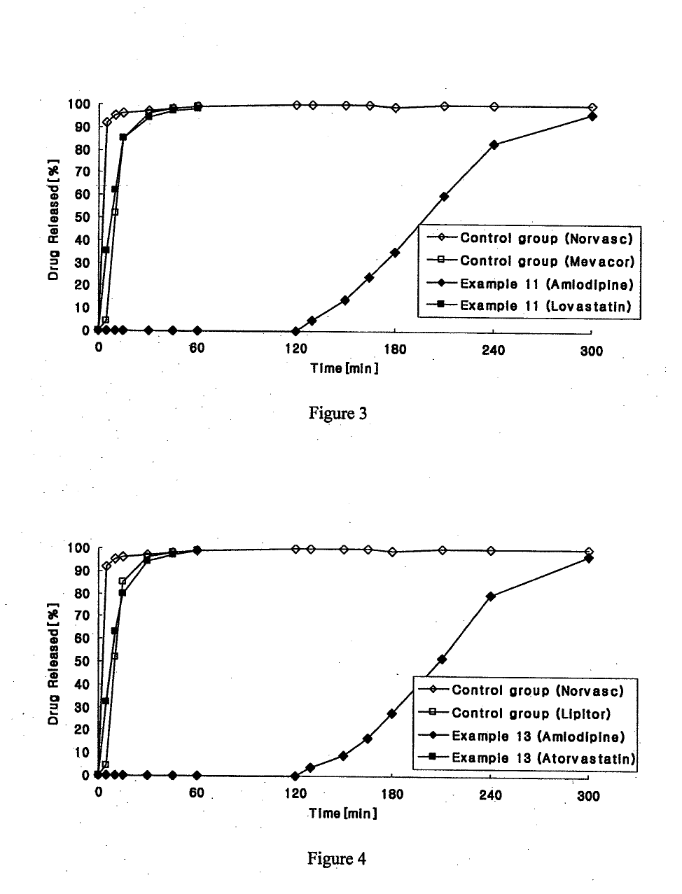

Comparative dissolution profile test was performed using an amlodipine/lovastatin combined pharmaceutical formulation prepared in Example 11 and control drugs (Mevacor®: lovastatin single pill, Norvasc®: amlodipine single pill, Pfizer). In the case of dissolution profile test of amlodipine ingredient, the dissolution solution was changed from an artificial gastric juice to an artificial intestinal juice after 2 hours. Specific process of dissolution profile test of each ingredient is described below, and the results are presented in FIG. 3.

When the dissolution profile test was performed under the conditions described below in Example 11, lovastatin ingredient showed a nearly equivalent dissolution behavior as compared to that of the control drug (Mevacor®), while amlodipine ingredient showed a very delayed dissolution rate as compared to that of the control drug (Norvasc®). In the case of the amlodipine/lovastatin multi-layered tablet according to the present invention, dissolution rates of amlodipine ingredient were all within 50% until one hour after the test began, which were far lower than that of the control drug (about 99%).

As described above, in the amlodipine/lovastatin multi-layered tablet according to the present invention, amlodipine shows a far lower initial dissolution rate than that of lovastatin unlike the control drugs (i.e., amlodipine single pill), and thus the amlodipine/lovastatin multi-layered tablet according to the present invention is less likely to be subject to the metabolism in the liver ahead of lovastatin.

Test Method for Amlodipine:

Based on the general dissolution test method described in Korea Pharmacopoeia (8th revision); Test method: Paddle method), 75 rpm; Dissolution medium: 0.01 M Hydrochloric acid, 750 mL (to 2 hr), pH 6.8 simulated intestinal fluid 1,000 mL (after 2 hr; Analysis method: UV-visible spectrophotometry (detected wavelength=maximum 240 nm).

Test Method for Lovastatin:

Based on the ‘Lovastatin tablet’ part in USP XXIX; Test method: Paddle method, 50 rpm; Dissolution solution: pH=7.0 buffer solution (0.01 M monobasic sodium phosphate solution containing sodium lauryl sulfate 2.0% wt/wt as surfactant), 900 mL; Analysis method: High performance liquid chromatography; Detected wavelength: 230 nm; Mobile phase: Acetonitrile: 0.02 M monobasic sodium phosphate buffer solution (pH=4.0): methanol=5:3:1; Column: Octadecyl silyl silica gel packed in a stainless steel tube of 4.6 mm (internal diameter) and 250 mm (length); Flow rate: 1.5 mL/minute.

### Experimental Example 4

### Comparative Dissolution Profile Test

Comparative dissolution profile test was performed using an amlodipine/atorvastatin combined pharmaceutical formulation prepared in Example 13 and control drugs (Lipitor®: atorvastatin single pill, Norvasc: amlodipine single pill, Pfizer). In the case of dissolution profile test of amlodipine ingredient, the dissolution solution was changed from an artificial gastric juice to an artificial intestinal juice after 2 hours. Specific process of dissolution profile test of each ingredient is described below, and the results are presented in FIG. 4.

When the dissolution profile test was performed under the conditions described below in Example 13, atorvastatin ingredient showed a nearly equivalent dissolution behavior as compared to that of the control drug (Lipitor®), while amlodipine ingredient showed a very delayed dissolution rate as compared to that of the control drug (Norvasc®). In the case of the amlodipine/atorvastatin multi-layered tablet according to the present invention, dissolution rates of amlodipine ingredient were all within 50% until one hour after the test began, which were far lower than that of the control drug (about 99%).

As described above, in the amlodipine/atorvastatin multi-layered tablet according to the present invention, amlodipine shows a far lower initial dissolution rate than atorvastatin unlike the control drugs (i.e., amlodipine single pill), and thus the amlodipine/atorvastatin multi-layered tablet according to the present invention is less likely to be subject to the metabolism in liver ahead of atorvastatin.

Test Method for Amlodipine:

Based on the general dissolution test method described in Korea Pharmacopoeia (8th revision); Test method: Paddle method), 75 rpm; Dissolution medium: 0.01 M Hydrochloric acid, 750 mL (to 2 hr), pH 6.8 simulated intestinal fluid 1,000 mL (after 2 hr); Analysis method: UV-visible spectrophotometry (detected wavelength=maximum 240 μm).

Test Method for Atorvastatin:

Based on the general dissolution test method described in Korea Pharmacopoeia (8th revision); Test method: Paddle method, 50 rpm; Dissolution solution: pH=7.0 buffer solution (0.01 M monobasic sodium phosphate solution containing sodium lauryl sulfate 2% wt/wt as surfactant), 900 mL; Analysis method: High performance liquid chromatography; Detected wavelength: 247 nm; Mobile phase: Methanol:0.025 M monobasic sodium phosphate buffer solution (pH=4.0): methanol=67:33 (pH=4.0); Column: Octadecyl silyl silica gel packed in a stainless steel tube of 4.6 mm (internal diameter) and 250 mm (length); Flow rate: 1.5 mL/minute

### Experimental Example 5

### Comparative Dissolution Profile Test of Lercanidine-Simvastatin Multi-Layered Tablets

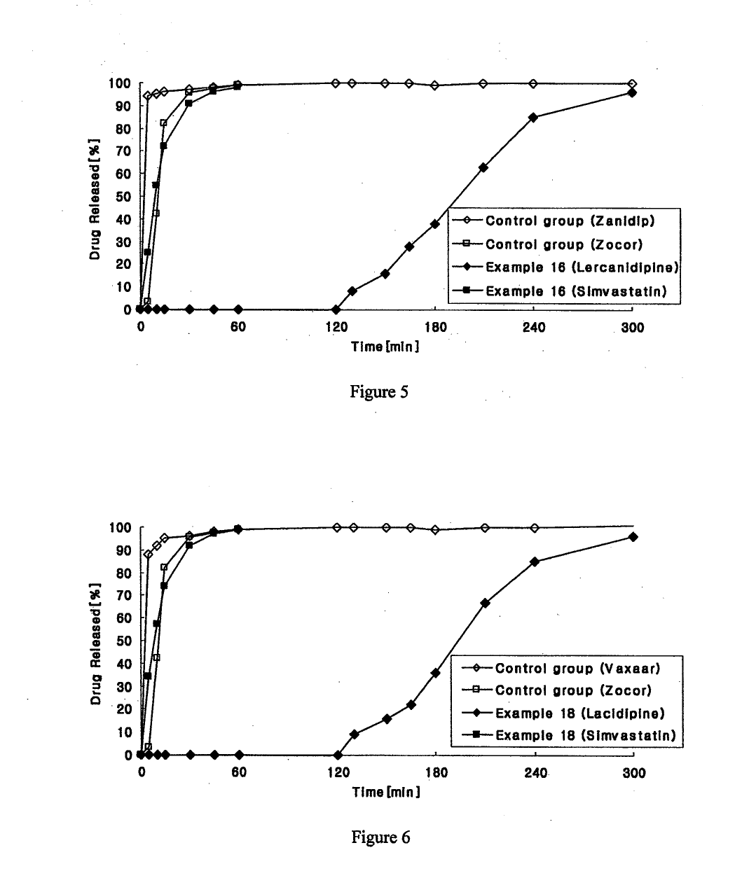

Comparative dissolution profile test was performed using a lercanidipine/simvastatin combined pharmaceutical formulation prepared in Example 16 and control drugs (Zocor®: simvastatin single pill, Zanidip®: lercanidipine single pill, LG Life Sciences Ltd.). In the case of dissolution profile test of lercanidipine ingredient, the dissolution solution was changed from an artificial gastric juice to an artificial intestinal juice after 2 hours. Specific process of dissolution profile test of each ingredient is described below, and the results are presented in FIG. 5.

When the dissolution profile test was performed under the conditions described below in Example 16, the simvastatin ingredient showed a nearly equivalent dissolution behavior as compared to that of the control drug (Zocor®), while the lercanidipine ingredient showed a very delayed dissolution rate as compared to that of the control drug (Zanidip®). In the case of the lercanidipine/simvastatin multi-layered tablet according to the present invention, dissolution rates of lercanidipine ingredient were all within 50% until one hour after the test began, which were far lower than that of the control drug (about 99%).

As described above, in the lercanidipine/simvastatin multi-layered tablet according to the present invention, lercanidipine shows a far lower initial dissolution rate than simvastatin unlike the control drugs (i.e., lercanidipine single pill), and thus the lercanidipine/simvastatin multi-layered tablet according to the present invention is less likely to be subject to the metabolism in the liver ahead of simvastatin.

Test Method for Lercanidipine:

Based on the general dissolution test method described in Korea Pharmacopoeia (8th revision); Test method: Paddle method), 75 rpm; Dissolution medium: 0.01 M Hydrochloric acid, 750 mL (to 2 hr), pH 6.8 simulated intestinal fluid 1,000 mL (after 2 hr); Analysis method: High performance liquid chromatography; Detected wavelength: 356 μm; Mobile phase: Acetonitrile: 0.01 M phosphate buffer solution=45:55 (pH=4.0); Column: Octadecyl silyl silica gel packed in a stainless steel tube of 4.6 mm (internal diameter) and 250 mm (length); Flow rate: 1.0 mL/minute.

Test Method for Simvastatin:

Based on the ‘Simvastatin tablet’ part in USP XXIX; Test method: Paddle method, 50 rpm; Dissolution solution: pH=7.0 buffer solution (0.01 M monobasic sodium phosphate solution containing sodium lauryl sulfate 0.5% wt/wt as surfactant), 900 mL; Analysis method: UV-visible spectrophotometry (detected wavelength=maximum 247 nm and minimum 257 nm)

### Experimental Example 6

### Comparative Dissolution Profile Test of Lacidipine-Simvastatin Multi Layered Tablet

Comparative dissolution profile test was performed using a lacidipine/simvastatin combined pharmaceutical formulation prepared in Example 18 and control drugs (Zocor®: simvastatin single pill, MSD, Vaxaar®: lacidipine single pill, GlaxoSmithkline Plc.). In the case of dissolution profile test of lacidipine ingredient, the dissolution solution was changed from artificial gastric juice to artificial intestinal juice after 2 hours. Specific process of dissolution profile test of each ingredient is described below, and the results are presented in FIG. 6.

When the dissolution profile test was performed under the conditions described below in Example 18, the simvastatin ingredient showed a nearly equivalent dissolution behavior as compared to that of the control drug (Zocor®), while the lacidipine ingredient showed a very delayed dissolution rate as compared to that of the control drug (Vaxaar®). In the case of the lacidipine/simvastatin multi-layered tablet according to the present invention, dissolution rates of lacidipine ingredient were all within 50% until one hour after the test began, which were far lower than that of the control drug (about 99%).

As described above, in the lacidipine/simvastatin multi-layered tablet according to the present invention, lacidipine shows a far lower initial dissolution rate than simvastatin unlike the control drugs (i.e., lacidipine single pill), and thus the lacidipine/simvastatin multi-layered tablet according to the present invention is less likely to be subject to the metabolism in the liver ahead of simvastatin.

Test Method for Lacidipine:

Based on the general dissolution test method described in Korea Pharmacopoeia (8th revision); Test method: Paddle method), 75 rpm; Dissolution medium: 0.01 M Hydrochloric acid, 750 mL (to 2 hr), pH 6.8 simulated intestinal fluid 1,000 mL (after 2 hr); Analysis method: High performance liquid chromatography; Detected wavelength: 282 μm; Mobile phase: Acetronitrile:0.05 M ammonium acetate buffer solution=80:20; Column: Octadecyl silyl silica gel packed in a stainless steel tube of 4.6 mm (internal diameter) and 250 mm (length); Flow rate: 1.0 mL/minute.

Test Method for Simvastatin:

Based on the ‘Simvastatin tablet’ part in USP XXIX; Test method: Paddle method, 50 rpm; Dissolution medium: pH=7.0 buffer solution (0.01 M monobasic sodium phosphate solution containing sodium lauryl sulfate 0.5% wt/wt as surfactant), 900 mL; Analysis method: UV-visible spectrophotometry (detected wavelength=maximum 247 nm and minimum 257 nm).

### Experimental Example 7

### Comparative Dissolution Profile Test of Amlodipine-Simvastatin Press-Coated Tablet

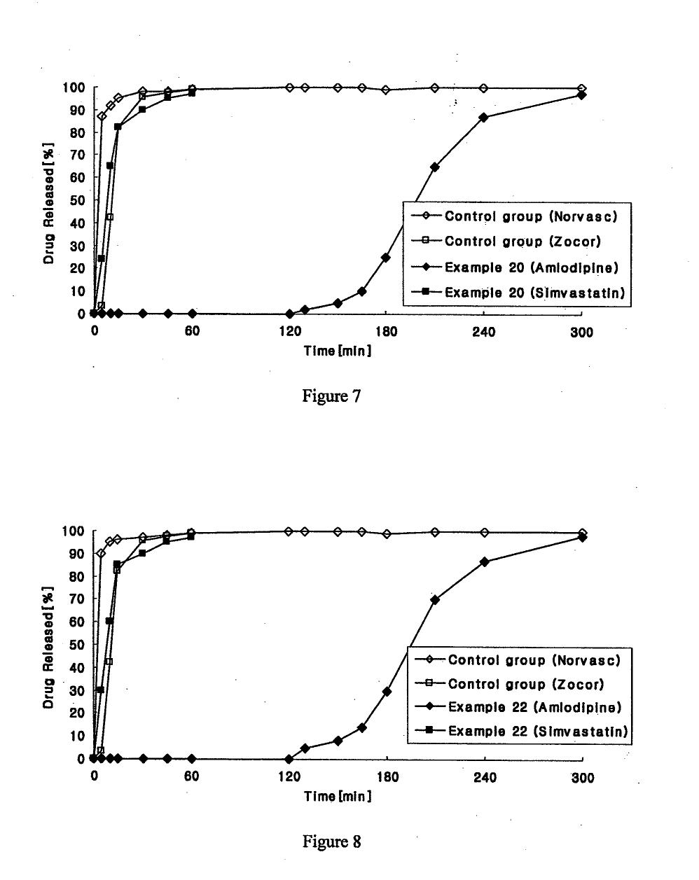

Comparative dissolution profile test was performed using an amlodipine/simvastatin press-coated tablets prepared in Example 20 and control drugs (Zocor®: simvastatin single pill, MSD, Norvasc: amlodipine single pill, Pfizer). In the case of dissolution profile test of amlodipine ingredient, the dissolution medium was changed from an simulated gastric fluid to an simulated intestinal fluid after 2 hours. Specific process of dissolution profile test of each ingredient is described below, and the results are presented in FIG. 7.

When the dissolution profile test was performed under the conditions described below, in the Press-coated tablets according to the present invention, the simvastatin ingredient showed a nearly equivalent dissolution behavior as compared to that of the control drug (Zocor®), while the amlodipine ingredient showed a very delayed dissolution rate as compared to that of the control drug (Norvasc®). In the case of the amlodipine/simvastatin press-coated tablets according to the present invention, dissolution rates of amlodipine ingredient were all within 50% until one hour after the test began, which were far lower than that of the control drug (about 99%).

As described above, in the amlodipine/simvastatin press-coated tablets according to the present invention, amlodipine shows a far lower initial dissolution rate than simvastatin unlike the control drugs (i.e., amlodipine single pill), so amlodipine is less likely to be subject to the metabolism in the liver ahead of simvastatin.

Test Method for Amlodipine:

Based on the general dissolution test method described in Korea Pharmacopoeia (8th revision); Test method: Paddle method, 75 rpm; Dissolution medium: 0.01M—Hydrochloric acid, 750 mL (to 2 hr); pH 6.8 simulated intestinal fluid 1,000 mL (after 2 hr); Analysis method: UV-visible spectrophotometry (detected wavelength=maximum 240 nm)

Test Method for Simvastatin:

Based on the ‘Simvastatin tablet’ part in USP XXIX, Test method: Paddle method, 50 rpm; Dissolution medium: pH=7.0 buffer solution (0.01M—monobasic sodium phosphate solution containing sodium lauryl sulfate 0.5% wt/wt as surfactant), 900 mL; Analysis method: UV-visible spectrophotometry (detected wavelength=maximum 247 nm and minimum 257 nm)

### Experimental Example 8

### Comparative Dissolution Profile Test of Amlodipine-Simvastatin Biphasic Capsule

Comparative dissolution profile test was performed using an amlodipine/simvastatin capsule containing combination products prepared in Example 22 and control drugs (Zocor®: simvastatin single pill, MSD, Norvasc: amlodipine single pill, Pfizer). In the case of dissolution profile test of amlodipine ingredient, the dissolution medium was changed from simulated gastric fluid to simulated intestinal fluid after 2 hours. The detailed process of dissolution profile test of each ingredient is described below, and the results are presented in FIG. 8.

When the dissolution profile test was performed under the conditions described below, in the capsule containing combination products according to the present invention, the simvastatin ingredient showed a nearly equivalent dissolution behavior as compared to that of the control drug (Zocor®), while the amlodipine ingredient showed a very delayed dissolution rate as compared to that of the control drug (Norvasc®). In the case of the amlodipine/simvastatin capsule containing combination products according to the present invention, dissolution rates of amlodipine ingredient were all within 50% until one hour after the test began, which were far lower than that of the control drug (about 99%).

As described above, in the amlodipine/simvastatin capsule containing combination products according to the present invention, amlodipine shows a far lower initial dissolution rate than simvastatin unlike the control drugs (i.e., amlodipine single pill), so amlodipine is less likely to be subject to the metabolism in the liver ahead of simvastatin.

Test Method for Amlodipine:

Based on the general dissolution test method described in Korea Pharmacopoeia (8th revision); Test method: Paddle method, 75 rpm; Dissolution medium: 0.01M—Hydrochloric acid, 750 mL (to 2 hr); pH 6.8 simulated intestinal fluid 1,000 mL (after 2 hr); Analysis method: UV-visible spectrophotometry (detected wavelength=maximum 240 nm).

Test Method for Simvastatin:

Based on the ‘Simvastatin tablet’ part in USP XXIX; Test method: Paddle method, 50 rpm; Dissolution medium: pH=7.0 buffer solution (0.01M—monobasic sodium phosphate solution containing sodium lauryl sulfate 0.5% wt/wt as surfactant), 900 mL; Analysis method: UV-visible spectrophotometry (detected wavelength=maximum 247 nm and minimum 257 nm)

### Experimental Example 9

### Comparative Dissolution Profile Test of Amlodipine-Atorvastatin Biphasic Capsule

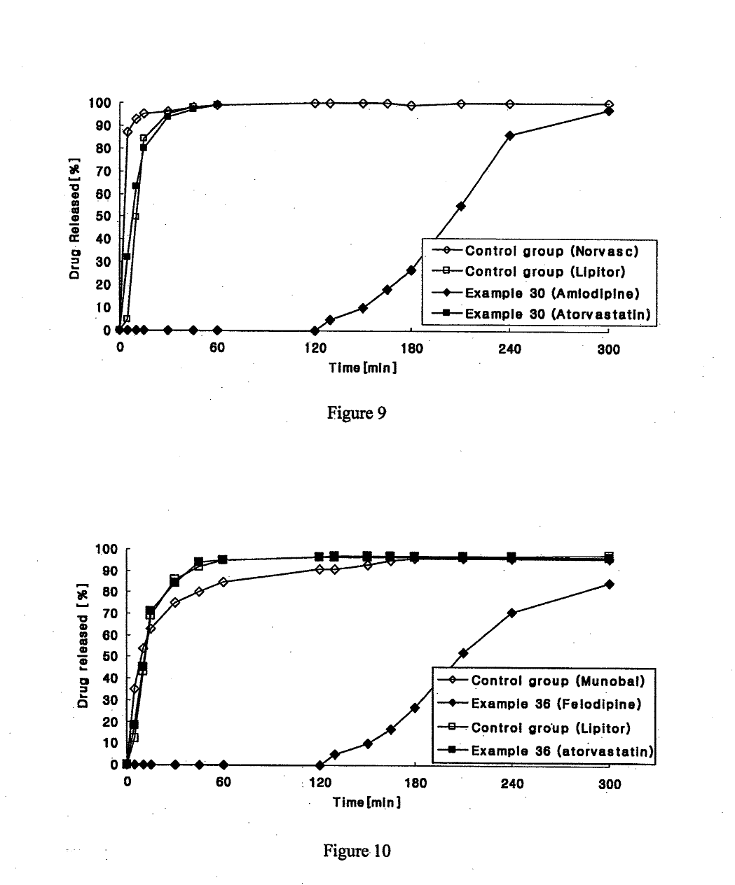

Comparative dissolution profile test was performed using an amlodipine/atorvastatin combined pharmaceutical formulation prepared in Example 30 and control drugs (Lipitor®: atorvastatin single pill, Norvasc: amlodipine single pill, Pfizer). In the case of dissolution profile test of amlodipine ingredient, the dissolution medium was changed from simulated gastric fluid to simulated intestinal fluid after 2 hours. The detailed process of dissolution profile test of each ingredient is described below, and the results are presented in FIG. 9.

When the dissolution profile test was performed under the conditions described below in Example 30, atorvastatin ingredient showed a nearly equivalent dissolution behavior as compared to that of the control drug (Lipitor®), while amlodipine ingredient showed a very delayed dissolution rate as compared to that of the control drug (Norvasc®). In the case of the amlodipine/atorvastatin combined pharmaceutical formulation according to the present invention, dissolution rates of amlodipine ingredient were all within 50% until one hour after the test began, which were far lower than that of the control drug (about 99%).

As described above, in the amlodipine/atorvastatin combined pharmaceutical formulation according to the present invention, amlodipine shows a far lower initial dissolution rate than atorvastatin unlike the control drugs (i.e., amlodipine single pill), and so amlodipine is less likely to be subject to the metabolism in the liver ahead of atorvastatin.

Test Method for Amlodipine:

Based on the general dissolution test method described in Korea Pharmacopoeia (8th revision); Test method: Paddle method), 75 rpm; Dissolution medium: 0.01M—Hydrochloric acid, 750 mL (to 2 hr); pH 6.8 simulated intestinal fluid 1,000 mL (after 2 hr); Analysis method: UV-visible spectrophotometry (detected wavelength=maximum 240 μm).

Test Method for Atorvastatin:

Based on the general dissolution test method described in Korea Pharmacopoeia (8th revision); Test method: Paddle method, 50 rpm; Dissolution medium: pH=7.0 buffer solution (0.01M—monobasic sodium phosphate solution containing sodium lauryl sulfate 2% wt/wt as surfactant), 900 mL; Analysis method: High performance liquid chromatography; Detected wavelength: 247 μm; Mobile phase: Methanol:0.025M—monobasic sodium phosphate buffer solution (pH=4.0)=67:33 (pH=4.0); Column: Octadecyl silyl silica gel packed in a stainless steel tube of 4.6 mm (internal diameter) and 250 mm (length); Flow rate: 1.5 mL/minute.

### Experimental Example 10

### Comparative Dissolution Profile Test of Felodipine-Atorvastatin Biphasic Capsule

Comparative dissolution profile test was performed using felodipine/atorvastatin two-phase capsules prepared in Example 36 and control drugs (Lipitor®: atorvastatin single pill, Munobal®: felodipine single pill, Handok). In the case of dissolution profile test of felodipine ingredient, the dissolution medium was changed from an simulated gastric fluid containing sodium lauryl sulfate as surfactant to an simulated intestinal fluid containing sodium lauryl sulfate as surfactant after 2 hours. Specific process of dissolution profile test of each ingredient is described below, and the results are presented in FIG. 10.

When the dissolution profile test was performed under the conditions described below in Example 36, atorvastatin ingredient showed a nearly equivalent dissolution behavior as compared to that of the control drug (Lipitor®), while felodipine ingredient showed a very delayed dissolution rate as compared to that of the control drug (Munobal®).

As described above, in the felodipine/atorvastatin two-phase capsules according to the present invention, felodipine shows a far lower initial dissolution rate than atorvastatin unlike the control drugs (i.e., felodipine single pill), and so felodipine is less likely to be subject to the metabolism in the liver ahead of atorvastatin.

Test Method for Felodipine:

Based on the general dissolution test method described in Korea Pharmacopoeia (8th revision); Test method: Paddle method), 50 rpm; Dissolution medium: 0.01M—Hydrochloric acid containing 2% Sodium lauryl sulfate, 750 mL (to 2 hr); pH 6.8 simulated intestinal fluid containing 2% Sodium lauryl sulfate, 1,000 mL (after 2 hr); Analysis method: UV-visible spectrophotometry (detected wavelength=maximum 254 nm).

Test Method for Atorvastatin:

Based on the general dissolution test method described in Korea Pharmacopoeia (8th revision); Test method: Paddle method, 50 rpm; Dissolution medium: pH=7.0 buffer solution (0.01M—monobasic sodium phosphate solution containing sodium lauryl sulfate 2% wt/wt as surfactant), 900 mL; Analysis method: High performance liquid chromatography; Detected wavelength: 247 nm; Mobile phase: Methanol:0.025M—monobasic sodium phosphate buffer solution (pH=4.0) 67:33 (pH=4.0); Column: Octadecyl silyl silica gel packed in a stainless steel tube of 4.6 mm (internal diameter) and 250 mm (length); Flow rate: 1.5 mL/minute.

### Experimental Example 11

### Comparative Dissolution Profile Test of Isradipine-Fluvastatin Biphasic Capsule

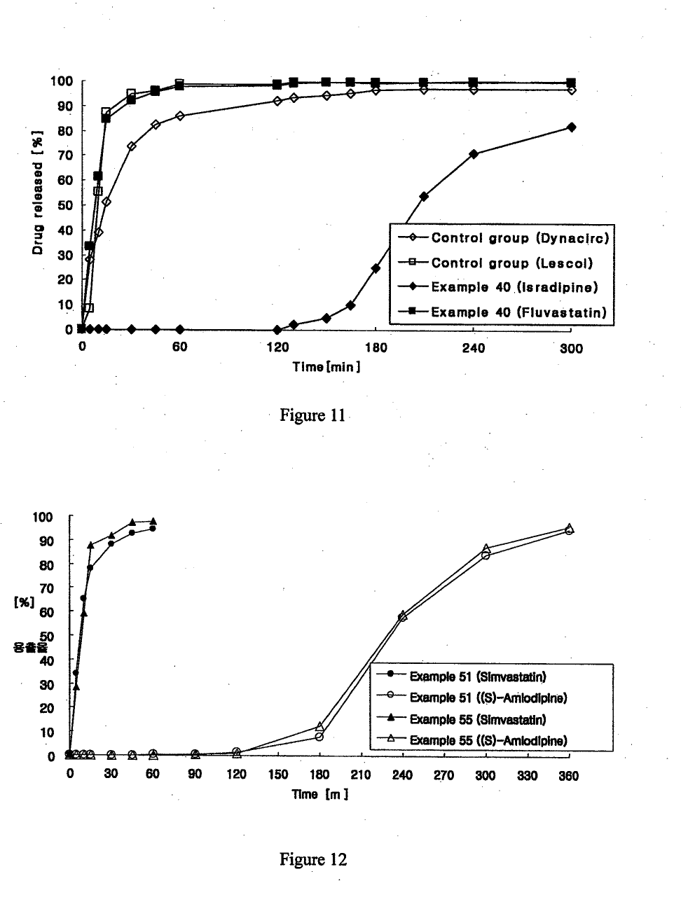

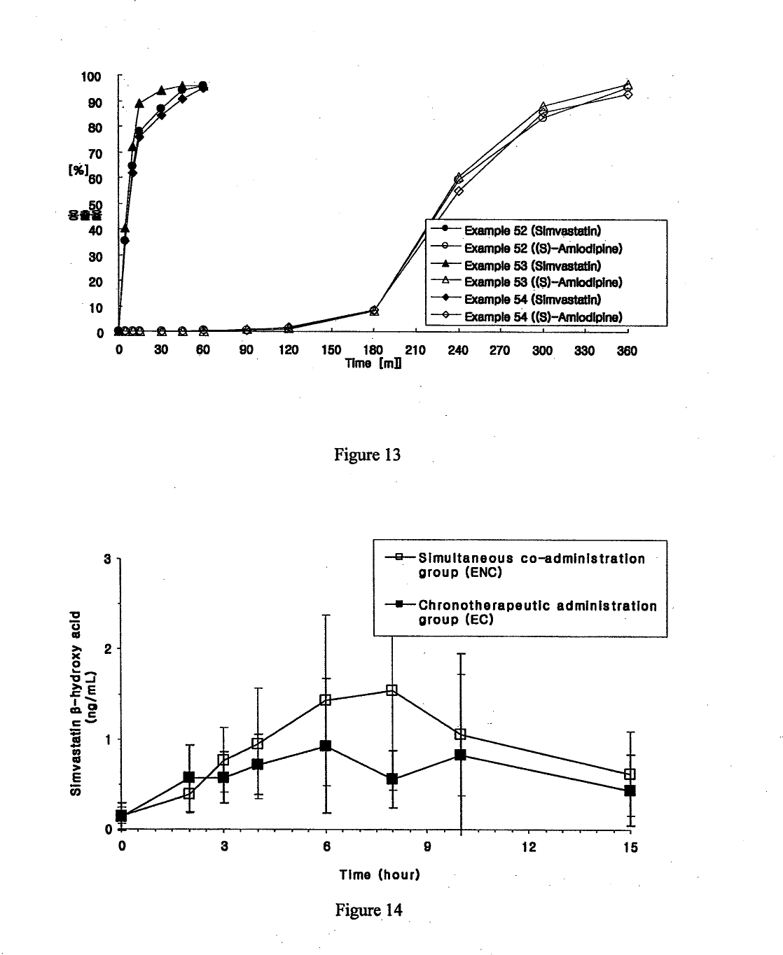

Comparative dissolution profile test was performed using isradipine/fluvastatin two-phase capsules prepared in Example 40 and control drugs (Lescol®: fluvastatin single pill, Dynacirc®: isradipine single pill, Reliant Pharm.). In the case of dissolution profile test of Isradipine ingredient, the dissolution medium was changed from simulated gastric fluid containing sodium lauryl sulfate as surfactant to an simulated intestinal fluid containing sodium lauryl sulfate as surfactant after 2 hours. Specific process of dissolution profile test of each ingredient is described below, and the results are presented in FIG. 11.

When the dissolution profile test was performed under the conditions described below in Example 40, fluvastatin ingredient showed a nearly equivalent dissolution behavior as compared to that of the control drug (Lescol®), while Isradipine ingredient showed a very delayed dissolution rate as compared to that of the control drug (Dynacirc®).

As described above, in the isradipine/fluvastatin two-phase capsules according to the present invention, isradipine shows a far lower initial dissolution rate than fluvastatin unlike the control drugs (i.e., felodipine single pill), and so isradipine is less likely to be subject to the metabolism in the liver ahead of fluvastatin.

Test Method for Isradipine:

Based on the general dissolution test method described in Korea Pharmacopoeia (8th revision); Test method: Paddle method), 50 rpm; Dissolution medium: 0.01M—Hydrochloric acid containing 2% Sodium lauryl sulfate, 750 mL (to 2 hr); pH 6.8 simulated intestinal fluid containing 2% Sodium lauryl sulfate, 1,000 mL (after 2 hr); Analysis method: UV-visible spectrophotometry (detected wavelength=maximum 328 nm).

Test Method for Fluvastatin:

Based on the “Fluvastatin capsules” part in USP XXX; Test method: Paddle method, 50 rpm; Dissolution medium: Water 900 mL; Analysis method: High performance liquid chromatography; Detected wavelength: 235 nm; Mobile phase: Methanol.Acetonitril mixture (3:2): 0.016M—Ammonium dihydrogen phosphate buffer solution (pH=3.5)=7:3, Column: Octadecyl silyl silica gel packed in a stainless steel tube of 4.6 mm (internal diameter) and 100 mm (length); Flow rate: 2.0 mL/minute.

### Experimental Example 12

### Disintegration Test of Amlodipine-Simvastatin Preparations

Disintegration test was performed using an (S)-amlodipine/simvastatin combined pharmaceutical formulation prepared in Example 50, 51, 52. The disintegration test method was followed by USP monograph ‘disintegration Test’ and the results are presented in Table 12.

While disintegration of the simvastatin layer was very fast under the test conditions in all Examples, the (S)-amlodipine layer was not disintegrated in the first disintegration medium excluding Example 50. The (S)-amlodipine layer was disintegrated in the second disintegration medium after a certain delay time.

As described above, when a patients were administered the (S)-amlodipine/simvastatin combined pharmaceutical formulation according to the present invention, simvastatin ingredient is first dissolved and absorbed, and then (S)-amlodipine ingredient is absorbed this shows that (S)-amlodipine is less likely to be subject to the metabolism in the liver ahead of simvastatin.

Test Method for Disintegration Test:

First disintegration medium: pH 1.2, 700 mL, 37±2° C.; second disintegration medium: pH 6.8, 700 mL, 37±2° C.

### Experimental Example 13

### Comparative Dissolution Profile Test of (S)-Amlodipine-Simvastatin Preparations

Comparative dissolution profile test was performed using an (S)-amlodipine/simvastatin combined pharmaceutical formulation prepared in Example 50, 51, 52, 53, 54. In the case of dissolution profile test of (S)-amlodipine ingredient, the dissolution medium was changed from an simulated gastric fluid to an simulated intestinal fluid after 2 hours. Specific process of dissolution profile test of each ingredient is described below, and the results are presented in FIG. 12, 13.

When the dissolution profile test was performed under the conditions described below, the simvastatin ingredient showed rapid dissolution behavior, while the (S)-amlodipine ingredient showed a very delayed dissolution rate. As a result (S)-amlodipine in combined pharmaceutical formulation according to the present invention is less likely to be subject to the metabolism in the liver ahead of simvastatin through delaying absorption time.

Test Method for (S)-Amlodipine:

Based on the general dissolution test method described in Korea Pharmacopoeia (8th revision); Test method: Paddle method, 75 rpm; Dissolution medium: 0.01 M—Hydrochloric acid, 750 mL (to 2 hr); pH 6.8 simulated intestinal fluid 1,000 mL (after 2 hr); Analysis method: UV-visible spectrophotometry (detected wavelength=maximum 240 nm).

Test Method for Simvastatin:

Based on the “Simvastatin tablet” part in USP XXIX; Test method: Paddle method, 50 rpm; Dissolution medium: pH=7.0 buffer solution (0.01M—monobasic sodium phosphate solution containing sodium lauryl sulfate 0.5% wt/wt as surfactant), 900 mL; Analysis method: UV-visible spectrophotometry (detected wavelength=maximum 247 nm and minimum 257 μm).

TABLE 12

| | | Disintegration test | | | |
|---|---|---|---|---|---|
| | | first disintegration medium | | second disintegration medium | |
| | | (pH 1.2) | | (pH 6.8) | |
| | | Disintegration | Disintegration | Disintegration | Disintegration |
| Drug layer | Sample | finished or not | time (Ave, min) | finished or not | time (Ave, min) |
| Simvastatin | Example 50 | yes | 21.2 | yes | 22.6 |
| layer | Example 51 | yes | 20.2 | yes | 21.8 |
| | Example 52 | yes | 19.6 | yes | 18.3 |
| | Example 54 | yes | 19.4 | yes | 17.9 |
| (S)-Amlodipine | Example 50 | yes | 71.5 | yes | 72.5 |
| layer | Example 51 | no | — | yes | About 240 |
| | Example 52 | no | — | yes | About 240 |
| | Example 54 | no | — | yes | About 240 |

### Experimental Example 14

### Clinical Study

A clinical study was conducted as described in Table 13 to compare the therapeutic effects of (i) simultaneous co-administration of Zocor® tablet (Simvastatin 20 mg, MSD) and ‘Norvasc® tablet’ (amlodipine besylate 6.944 mg, Pfizer) and, as an experimental group, (ii) a chronotherapeutic administration of Zocor® tablet and Norvasc® tablet. The chronotherapeutic administration was so designed that the release time of drugs are the same as in a combined formulation according to the present invention.

TABLE 13

| Title | Multicenter trial (academic, research initiated trial of investigator) for | | |
|---|---|---|---|
| | comparing the pharmacokinetic characteristics, efficacy and safety in (i) a | | |
| | simultaneous co-administration and (ii) a chronotherapeutic administration | | |
| | amlodipine and simvastatin to subjects with hypertension and hyperlipidemia | | |
| Objectives | Comparative evaluation of steady-state pharmacokinetic characteristics, | | |
| | efficacy and safety in a simultaneous co-administration and a | | |
| | chronotherapeutic administration of amlodipine and simvastatin in the | | |
| | evening | | |
| Number of | Eight male and female adults aged 30-75 with hypertension and | | |
| subjects | hyperlipidemia per each group (total 16 subjects) | | |
| Mothodology | This clinical study is designed as follows. | | |
| | 2 open-label, single dose, randomized | | |
| | Drug 1: Norvasc ® 5 mg (single pill) | | |
| | Drug 2: Zocor ® 20 mg (single pill) | | |
| | Sixteen subjects are divided into 2 groups, i.e., (i) a simultaneous co- | | |
| | administration group of Norvasc ® and Zocor ® in the evening and (ii) a | | |
| | chronotherapeutic administration group of Norvasc ® and Zocor ® in the | | |
| | evening. Each subject is administered for 6 weeks (42 days), and the | | |
| | pharmacokinetic characteristics, efficacy and safety of the two groups are | | |
| | compared. | | |
| Evaluation | 1. Evaluation of efficacy | | |
| | Primary evaluation: Normalization of the mean systolic blood pressure | | |
| | measured with an automatic blood pressure meter and LDL-C are compared | | |
| | between a simultaneous co-administration group and a chronotherapeutic | | |
| | administration group after the study is finished. | | |
| | Secondary evaluation: Normalization of the mean diastolic blood pressure | | |
| | measured with an automatic blood pressure meter, pulse pressure, lipid | | |
| | profiles (total cholesterol (mg/dl), LDL-cholesterol (mg/dl), HDL-cholesterol | | |
| | (mg/dl) and triglyceride (mg/dl) are compared between a simultaneous co- | | |
| | administration group and a chronotherapeutic administration group after the | | |
| | study is finished. | | |
| | 2. Evaluation of drug concentration | | |
| | Plasma concentrations of simvastatin, simvastatin β-hydroxy acid and | | |
| | amlodipine are measured after administration by using LC/MS/MS. Two | | |
| | groups are compared in terms of the following parameters: | | |
| | Area under the plasma concentration-time curve: AUC_{0-∞}, AUC_{0-tz}, AUC_{0-∞} | | |
| | Maximum plasma concentration: Cₘₐₓ | | |
| | Elimination half-life: T_{1/2} | | |
| | | | Subject |
| | Group | Drug and administration | number |
| Study | Chronotherapeutic | Simvastatin (20 mg) at 7 p.m. | 8 |
| group | administration | Amlodipine (5 mg) at 10-11 p.m. | |
| | Simultaneous co- | Simvastatin (20 mg) and amlodipine (5 mg) | 8 |
| | administration | at 7 p.m. | |

This test was conducted for ascertaining the effect of the present invention by using the standards of domestic and foreign clinical studies for medication approval and reducing the numbers of experimental groups. However, test subjects were strictly controlled, and the standards of clinical test were rigidly followed during the test. Comparative results of the clinical test are presented in Table 14 and FIGS. 14-16.

TABLE 14
Clinical study for comparing a chronotherapeutic administration and
a simultaneous co-administration (Korea University Medical Center)

| 1 | Disease | Hypertension with | | Hypertension with | |
|---|---|---|---|---|---|
| | | hyperlipidemia | | hyperlipidemia | |
| 2 | Group | Chronotherapeutic | | Simultaneous | |
| | | administration | | co-administration | |
| | | group (EC) | | group (ENC) | |
| 3 | Number of subject | 7 | | 9 | |
| | | After daily administration | | | |
| | | for 41 days | | | |
| 4 | Systolic blood pressure | 121 | mmHg | 127 | mmHg |
| 5 | Diastolic blood | 80 | mmHg | 82 | mmHg |
| | pressure | | | | |
| 6 | Mean blood pressure | 94 | mmHg | 97 | mmHg |
| 7 | Pulse pressure | 40 | mmHg | 45 | mmHg |
| 8 | Total cholesterol | 158 | mg/dl | 164 | mg/dl |
| 9 | LDL | 88 | mg/dl | 94 | mg/dl |
| 10 | Neutral lipid | 107 | mg/dl | 143 | mg/dl |
| 11 | HDL | 61 | mg/dl | 46 | mg/dl |
| 12 | S-GPT | 23 | IU/L | 39 | IU/L |
| 13 | S-GOT | 23 | IU/L | 38 | IU/L |
| 14 | CPK | 71 | IU/L | 85 | IU/L |
| 15 | r-GPT | 38 | IU/L | 46 | IU/L |
| 16 | Alkaline Phosphatase | 70 | IU/L | 57 | IU/L |

| | | | |
|---|---|---|---|
| | | (No significant | (No significant |
| | | change) | change) |
| 17 | Clinical adverse effect | One case (Diarrhea) | One case (Fatigue) |
| | | | One case (Cold) |
| 18 | Simvastatin β-hydroxy | 14.21 | 23.4 |
| | acid | | |
| | AUC (ng · hr/mL) | | |
| 19 | Simvastatin + | 38.50 | 43.73 |
| | Simvastatin β-hydroxy | | |
| | acid | | |
| | AUC (ng · hr/mL) | | |
| 20 | Amlodipine AUC₀₋₁₅ | 111.38 | 107.11 |
| | (ng · hr/mL) | | |

The chronotherapeutic administration group showed a lower systolic and diastolic blood pressure on the 41st day of daily administration than the simultaneous co-administration group.

The chronotherapeutic administration group showed a lower level of total cholesterol and low density lipoprotein (LDL) cholesterol on the 41st day of daily administration than the simultaneous co-administration group.

The chronotherapeutic administration group showed a higher activity of lowering the level of triglyceride, the most important lipid associated with hyperlipidemia, while the simultaneous co-administration group showed no activity of lowering triglyceride. Although statins-based agents can inhibit the synthesis of cholesterol, it is known to inhibit the synthesis of triglyceride by 8-25% because the synthesis of cholesterol is a reaction rate determining step in the synthesis of lipoprotein. The chronotherapeutic administration group lowers the synthesis of triglyceride by 20% and the simultaneous co-administration group showed no activity of lowering the synthesis of neutral lipid. One reason could be that the unnecessary higher blood concentration of simvastatin in the simultaneous co-administration group due to the antagonistic effects incurred by amlodipine on the hepatic enzymes in the liver seemed also to result in the lower control on the synthesis of triglyceride.

It is well known that HDL value should be increased for improving lipid-related diseases. The chronotherapeutic administration group showed a higher increase in HDL value.

Safety of drug formulation was evaluated by observing change in biomarkers on the 41st day of daily administration, and the results are as follows.
1) The chronotherapeutic administration showed the more favorable safety biomarkers in terms of S-GPT, S-GOT, CPK, γ-GPT and alkaline phosphatase values.
2) The simultaneous co-administration group was higher than the chronotherapeutic administration group in the concentration of simvastatin in plasma causing unnecessary inflammation caused by simvastatin.

Only two subjects showed three cases of side effects in the total two groups. Only one case of diarrhea was observed in the chronotherapeutic administration group, and two cases (fatigue and cough) were observed in the simultaneous co-administration group. Plasma concentration of drugs was changed as follows.
1) Plasma concentration of simvastatin β-hydroxy acid:
The chronotherapeutic administration was lower in plasma concentration of simvastatin β-hydroxy acid by 40% than the simultaneous co-administration (Table 15 and FIG. 14). This pharmacokinetic data shows that the chronotherapeutic administration is superior in lowering lipid to the simultaneous co-administration. (It implies that higher concentration of active simvastatin β-hydroxy acid than inactive simvastatin are consumed in the liver to inhibit cholesterol synthesis and are further completely metabolized in the liver to be excreted through bile duct but do not entering into the blood.)

TABLE 15
Comparison of AUC and plasma concentration
changes of simvastatin β-hydroxy acid

| Simvastatin β-hydroxy | AUC_{INF} (ng · hr/mL) | | Increase in plasma |
|---|---|---|---|
| acid | Average | Deviation | concentration (%) |
| Simultaneous co- | 23.44 | 12.52 | — |
| administration (ENC) | | | |
| Chronotherapeutic | 14.21 | 7.67 | −39.38 |
| administration (EC) | | | |

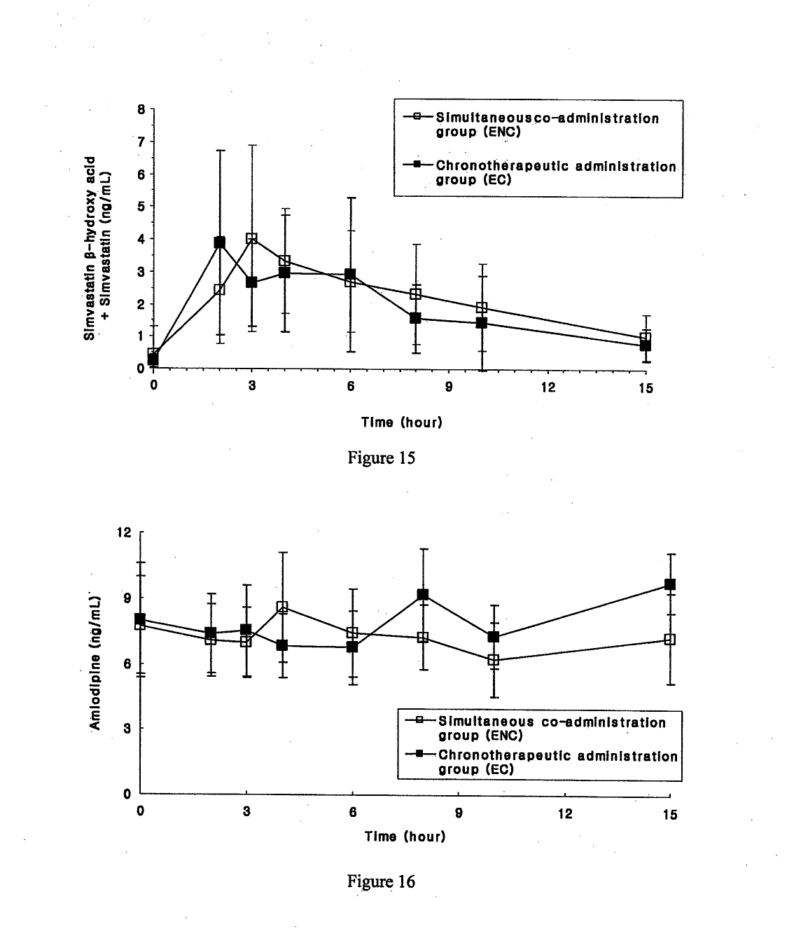

2) The total plasma concentration of simvastatin and simvastatin β-hydroxy acid as provided in Table 16 and FIG. 15 shows that chronotherapeutic administration has a lower blood concentration of total simvastatins, meaning that the highest concentration of them is consumed in the liver and therefore shows the most effective lipid-lowering figures and better biomarkers (to judge the possibility of side effect).

TABLE 16
Comparison of AUC and plasma concentration changes of total
Simvastatin (Simvastatin and Simvastatin β-hydroxy acid)

| Simvastatin + | AUC_{INF} | | |
|---|---|---|---|
| Simvastatin | (ng · hr/mL) | | Increase in plasma |
| β-hydroxy acid | Average | Deviation | concentration (%) |
| Simultaneous co- | 43.74 | 22.92 | — |
| administration (ENC) | | | |
| Chronotherapeutic | 38.50 | 13.40 | −11.98 |
| administration (EC) | | | |

3) Plasma concentration of amlodipine (Table 17): Plasma concentration of amlodipine is closely related to the activity of lowering the blood pressure, and also to the lipid-lowering activity because amlodipine has indirect activity in lowering the lipid level by improving atherosclerosis.

TABLE 17
Comparison of AUC and plasma concentration changes of amlodipine

AUC₀₋₁₅
(ng · hr/mL)

| Amlodipine | Average | Deviation |
|---|---|---|
| Simultaneous co- | 107.11 | 20.38 |
| administration (ENC) | | |
| Chronotherapeutic | 111.38 | 17.73 |
| administration (EC) | | |

As shown in FIGS. 14-15, the chronotherapeutic administration with time interval (Simvastatin first dissolved and absorbed in the liver and then 3-4 hours later amlodipine following the same course) makes it possible that simvastatin β-hydroxy acids as active forms could be fully formed from inactive simvastatin by the hepatic enzyme Cytochrome P450 3A4 and fully utilized in the liver and then further metabolized by the same hepatic enzymes to be excreted through the bile duct.

Therefore not showing unnecessary higher blood concentration of simvastatin β-hydroxy acids as shown in the simultaneous co-administration group, where the hepatic same enzymes Cytochrome P450 3A4 are inhibited to be induced by amlodipine at the presence of simvastatin in the liver, therefore the metabolism of simvastatin is partial, some of the formed simvastatin β-hydroxy acids are not further metabolized after acting.

And then flow into the blood to cause the unnecessary high blood concentration of simvastatin β-hydroxy acids, which could be the source of developing side effect, such muscular disorder like rhabdomyolysis.

Further, as shown in FIG. 16, Tmax (time to reach maximum plasma concentration) of dihydropyridine calcium channel blockers represented by amlodipine was delayed for about 4-5 hours in the chronotherapeutic administration group, and dihydropyridine calcium channel blockers show a delayed activity of lowering blood pressure as if administered at 12 in the evening. As a result, unlike a normal simultaneous co-administration group, a chronotherapeutic combined formulation may have a maximized activity of lowering blood pressure during the time between the morning and the noon next day, when mean blood pressure is highest.

As described above, the chronotherapeutic administration, designed as a combined formulation herein of dihydropyridine calcium channel blockers and statins, lipid-lowering agents, inhibit the side effects of statins, lipid-lowering agents as compared to the simultaneous co-administration. The chronotherapeutic administration herein also enables dihydropyridine calcium channel blockers, to demonstrate its maximized clinical effects in lowering blood pressure.

Table 18 compares the chronotherapeutic administration with the simultaneous co-administration in lipid-lowering efficacy. The chronotherapeutic administration group of Norvasc tablet (amlodipine besylate 5 mg) and Zocor tablet (simvastatin 20 mg) to be taken with time interval in the evening more remarkably decrease the plasma concentration of LDL-cholesterol and total cholesterol than the simultaneous co-administration group.

The combined products of the present invention may well be expected to demonstrate the same result that the drug component simvastatin transforms into an activated form in the liver by hepatic enzyme cytochrome P450 and fully exerts pharmacological action in the liver and fully metabolized by the same hepatic enzyme to be excreted through the bile duct, without being antagonistically affected by amlodipine.

TABLE 18
Comparison of total cholesterol, HDL, LDL and neutral lipid levels

| | | Chronotherapeutic | Simultaneous |
|---|---|---|---|
| | | administration | co-administration |
| Group | | group (EC) | group (ENC) |
| Total | Screening | 225 ± 28.0 | 239 ± 52.9 |
| cholesterol | D41 | 158 ± 25.1** | 164 ± 32.9** |
| HDL | Screening | 61 ± 18.0 | 48 ± 13.6 |
| | D41 | 61 ± 20.5 | 46 ± 7.6 |
| LDL | Screening | 153 ± 14.6 | 161 ± 43.1 |
| | D41 | 88 ± 11.8** | 94 ± 31.0** |
| Triglyceride | Screening | 135 ± 69.3 | 150 ± 57.3 |
| | D41 | 107 ± 29.1 | 143 ± 65.2 |

(* p < 0.05, **p < 0.01 vs. screening)

Tables 19 and 20 show the results relating to the blood pressure and pulse pressure, respectively, of the simultaneous co-administration group and the chronotherapeutic administration group. This ascertains that mean sitting systolic blood pressure and mean sitting diastolic blood pressure are significantly reduced in chronotherapeutic administration group. It surely is due to the high blood concentration of amlodipine (FIG. 16) in chronotherapeutic administration group.

As a result, the chronotherapeutic administration group shows superiority compared to the simultaneous co-administration group in lowering the blood pressure by delaying the release of amlodipine for about 4 hours.

TABLE 19
Comparison of blood pressure changes

| | | Chronotherapeutic | Simultaneous co- |
|---|---|---|---|
| | | administration group | administration group |
| Group | | (EC) | (ENC) |
| Systolic blood | Screening | 148 ± 6.3 | 150 ± 5.9 |
| pressure (mmHg) | D41 | 121 ± 8.8** | 127 ± 7.1** |
| | D42 | 117 ± 10.2** | 125 ± 8.8** |
| Diastolic blood | Screening | 95 ± 4.0 | 95 ± 2.8 |
| pressure (mmHg) | D41 | 80 ± 5.4** | 82 ± 8.1** |
| | D42 | 74 ± 8.2** | 79 ± 7.3** |
| Mean blood | Screening | 113 ± 4.5 | 113 ± 3.6 |
| pressure (mmHg) | D41 | 94 ± 5.8** | 97 ± 7.1** |
| | D42 | 88 ± 8.6** | 94 ± 7.5** |

(* p < 0.05, **p < 0.01 vs. screening)

TABLE 20
Comparison of pulse pressure and pulse frequency changes

| | | Chronotherapeutic | Simultaneous co- |
|---|---|---|---|
| | | administration group | administration group |
| Group | | (EC) | (ENC) |
| Pulse pressure | Screening | 53 ± 4.3 | 55 ± 4.2 |
| (mm/Hg) | D41 | 40 ± 7.1** | 45 ± 7.0* |
| | D42 | 43 ± 4.6** | 46 ± 4.6** |
| Pulse frequency | Screening | 75 ± 16.2 | 63 ± 10.1 |
| (bpm) | D41 | 73 ± 14.8 | 72 ± 11.5* |
| | D42 | 76 ± 12.5 | 68 ± 10.5 |

(*p < 0.05, **p < 0.01 vs. screening)

In conclusion, the clinical study above ascertains that chronotherapeutically combined formulation of dihydropyridine calcium channel blockers and statins, lipid-lowering agents according to the present invention shall synergistically demonstrate remarkable improvement in inhibiting high cholesterol by statins, lipid-lowering agent and lowering blood pressure by dihydropyridine calcium channel blockers at the same dosage as compared to a simultaneous co-administration of the two drugs, by means of the effects of release time control of each drug components.

As mentioned above, the present invention permits the combined product of two drugs to demonstrate chronotherapeutic effects and synergistic effects in improving efficacy in better way and safety in less way. Accordingly the control release formulation of the present invention is based on theory of xenobiotics and chronotherapy.

The formulation of the present invention comprises as active ingredients, statins, lipid-lowering agents, and dihydropyridine calcium channel blockers both of which are affected by to the same cytochrome P450 enzymes, in such a manner that one is affecting on while the other is affected by these enzymes, and is characterized in that the release rates of the aforementioned ingredients are different and the dissolution and absorption of each drug are initiated at certain intervals of time in a controlled manner. As a result, the formulation of the present invention is more useful pharmacologically, clinically, scientifically and economically in the treatment of a chronical circulatory disorder than when the two drugs are simultaneously co-administered without definitive time interval without consideration of the chronotherapeutic rhythm pattern.

The combination pharmaceutical formulation of the present invention causes drugs to be released at different rates, thereby preventing the antagonistic effects and lessening side effects, while maintaining the synergistic effect of the drugs.

Furthermore, the combined pharmaceutical formulation of the present invention is administered once daily as a single dose, thus providing convenience in medication and saving time of prescribers for patient instruction.

Having now fully described this invention, it will be understood to those of ordinary skill in the art that the compositions and methods of the present invention can be made and used with a wide and equivalent range of conditions, formulations, and other parameters without departing from the scope of the invention or any embodiments thereof.

What we claim is:

1. A pharmaceutical composition comprising a delayed release component comprising at least one dihydropyridine and a rapid release component comprising at least one statin.

2. The composition of claim 1 wherein the at least one dihydropyridine comprises at least one of amlodipine, lercanidipine, lacidipine, felodipine, barnidipine, benidipine, cilnidipine, isradipine, manidipine, nicardipine, nifedipine, nimodipine, nilvadipine, nisoldipine, nitrendipine, or a pharmaceutically acceptable salt, ester, or isomer thereof.

3. The composition of claim 1 wherein the at least one statin comprises at least one of simvastatin, lovastatin, atorvastatin, pitavastatin, rosuvastatin, fluvastatin, pravastatin or a pharmaceutically acceptable salt, ester, or isomer thereof.

4. The composition of claim 2 wherein the at least one statin comprises at least one of simvastatin, lovastatin, atorvastatin, pitavastatin, rosuvastatin, fluvastatin, pravastatin or a pharmaceutically acceptable salt, ester, or isomer thereof.

5. The composition of claim 1 comprising about 1-400 mg of the at least one dihydropyridine, and about 1-500 mg of the at least one statin.

6. The composition of claim 1, wherein the composition is in the form of a capsule, a tablet, or a combination thereof.

7. The composition of claim 1 in the form of a capsule comprising the at least one dihydropyridine, and the at least one statin.

8. The composition of claim 7 wherein the delayed release component comprises delayed release pellets.

9. The composition of claim 8 wherein the delayed release pellets include an enteric polymer.

10. The composition of claim 7 wherein the delayed release component comprises delayed release granules.

11. The composition of claim 7 wherein the delayed release component comprises a delayed release tablet.

12. The composition of claim 11 wherein the delayed release tablet comprises a pressed tablet.

13. The composition of claim 11 wherein the delayed release tablet comprises an osmotic pump.

14. The composition of claim 7 wherein the rapid release component comprises rapid release pellets.

15. The composition of claim 7 wherein the rapid release component comprises rapid release granules.

16. The composition of claim 1 wherein the delayed release component comprises at least one water insoluble polymer.

17. The composition of claim 16 wherein the at least one water-insoluble polymer comprises at least one of a polyvinylacetate, a methacrylic acid copolymer comprising at least one of poly(ethylacrylate-co-methylmethacrylate) copolymer or poly(ethylacrylate-methyl methacrylate-trimethyl aminoethyl methacrylate) copolymer, an ethyl cellulose, or a cellulose acetate.

18. The composition of claim 1 in the form of a tablet comprising a tablet comprising the at least one dihydropyridine, and the at least one statin.

19. The composition of claim 18 wherein the tablet comprises a delayed release core comprising the at least one dihydropyridine, and a rapid release coat on the tablet core, the rapid release coat comprising the at least one statin.

20. The composition of claim 19 wherein the delayed release core comprises an enteric coat comprising at least one enteric polymer.

21. The composition of claim 20 wherein the at least one enteric polymer comprises at least one of polyvinylacetate phthalate, methacrylic acid copolymer, hydroxypropylmethyl cellulose phthalate, shellac, cellulose acetate phthalate, cellulose propionate phthalate, poly(methacrylate, methylmethacrylate)polymer, or poly(methacrylate, ethylacrylate) polymer.

22. The composition of claim 18, wherein the delayed release component comprises an osmotic device.

23. The composition of claim 22 wherein the osmotic device comprises an osmotic agent.

24. The composition of claim 23 wherein the osmotic agent comprises at least one of magnesium sulfate, magnesium chloride, sodium chloride, lithium chloride, potassium sulfate, sodium sulfate or lithium sulfate.

25. The composition of claim 22, wherein the rapid release component comprises a rapid release layer or coat on the osmotic device.

26. A kit comprising the composition of claim 1 wherein the at least one dihydropyridine is contained in a first unit dosage form, and the at least one statin is contained in a second unit dosage form, and wherein the first unit dosage form and the second unit dosage forms are included in a package structured and designed to facilitate administering the first unit dosage form and second unit dosage forms at or about the same time.

27. The kit of claim 26, wherein the package comprises a blister package comprising at least one blister containing both of the first unit dosage form and second unit dosage form.

28. The kit of claim 26, wherein the package comprises an envelope containing both of the first unit dosage form and second unit dosage form.

29. The composition of claim 1 wherein at least about 80% of the at least one statin is released at about 1 hour, and no more than about 40% of the at least one dihydropyridine is released at about 3 hours when the composition is tested under the general dissolution test method of Korea Pharmacopoeia (8th revision); paddle method, 75 rpm; in dissolution medium 750 ml 0.01 M hydrochloric acid for two hours; and in dissolution medium 1000 mL of pH 6.8 artificial intestinal fluid (pH==6.8) thereafter.

30. The composition claim 1, wherein, upon administration to a human, the at least one dihydropyridine is absorbed in the liver at least about 3 hours after the at least one statin.

31. A pharmaceutical formulation comprising at least one statin and at least one dihydropyridine, the formulation being structured and arranged to provide release of the at least one dihydropyridine following a time interval after release of the at least one statin.

32. A method of administering to a patient in need thereof at least one statin and at least one dihydropyridine, the method comprising administering to the patient a formulation comprising a therapeutically effective amount of at least one statin and a therapeutically effective amount of at least one dihydropyridine, wherein the statin is absorbed, and after a time interval, the dihydropyridine is absorbed.

33. The method of claim 32 wherein the formulation is administered in the evening or at night.

34. The method of claim 32 wherein the composition is administered at bedtime.

35. The method of claim 32 wherein the composition is administered between about 5 pm and 10 pm.

36. A method of preventing or treating hypertension, angina pectoris, atherosclerosis, arteriosclerosis, complex hypertension, hyperlipidemia, hypercholesterolemia, myocardial infarction, cardioplegia, heart failure or ischemic heart disease, comprising administering to a patient in need thereof a pharmaceutical composition comprising a delayed release component comprising at a therapeutically effective amount of least one dihydropyridine and a rapid release component comprising a therapeutically effective amount of at least one statin.

37. A method of administering a drug combination comprising administering to a subject in need thereof a therapeutically effective amount of at least one statin and a therapeutically effective amount of at least one dihydropyridine wherein the statin is in a rapid-release form and the dihydropyridine is in a delayed-release form.

38. A method of delivering a statin and a dihydropyridine with a time differential to the liver of a subject, the method comprising administering to a subject in need thereof a therapeutically effective amount of at least one statin and a therapeutically effective amount of at least one dihydropyridine wherein the statin is in a rapid-release form and the dihydropyridine is in a delayed-release form.

39. A method of reducing interference between a statin and a dihydropyridine in a subject in need thereof comprising administering to the subject a therapeutically effective amount of at least one statin and a therapeutically effective amount of at least one dihydropyridine wherein the at least one statin is in a rapid-release form and the at least one dihydropyridine is in a delayed-release form.
